SEQUENCE LISTING (113 sequences)

SEQ ID NO: 1 - DNA, 33 bp, synthetic construct
  misc_feature 1..33: note = Synthetic
  source 1..33: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..32: note = RNA
  misc_feature 33: note = DNA
ctcttcgtcgctgtctccgcttcttcctgccat

SEQ ID NO: 2 - RNA, 46 nt, synthetic construct
  misc_feature 1..46: note = Synthetic
  source 1..46: mol_type = other RNA
gagacagcgacgggtaactagagatccctcagacgaggaagaagcg

SEQ ID NO: 3 - RNA, 25 nt, synthetic construct
  misc_feature 1..25: note = Synthetic
  source 1..25: mol_type = other RNA
cgtctgagggatctctagttacctt

SEQ ID NO: 4 - DNA, 31 bp, synthetic construct
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA
ctcttcgtcgctgtctccgcttcttcctgcc

SEQ ID NO: 5 - RNA, 46 nt, synthetic construct
  misc_feature 1..46: note = Synthetic
  source 1..46: mol_type = other RNA
gcggagacagcgggtaactagagatccctcagacgggcaggaagaa

SEQ ID NO: 6 - RNA, 25 nt, synthetic construct
  misc_feature 1..25: note = Synthetic
  source 1..25: mol_type = other RNA
cgtctgagggatctctagttacctt

SEQ ID NO: 7 - DNA, 31 bp, synthetic construct
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA
gacttctccagctcatggacctccatttcct

SEQ ID NO: 8 - RNA, 46 nt, synthetic construct
  misc_feature 1..46: note = Synthetic
  source 1..46: mol_type = other RNA
gctggtgaagtcggtaactagagatccctcagacggaggtccatga

SEQ ID NO: 9 - RNA, 25 nt, synthetic construct
  misc_feature 1..25: note = Synthetic
  source 1..25: mol_type = other RNA
cgtctgagggatctctagttacctt

SEQ ID NO: 10 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 11 - RNA, 46 nt, synthetic construct; identical to SEQ ID NO: 8
  misc_feature 1..46: note = Synthetic
  source 1..46: mol_type = other RNA

SEQ ID NO: 12 - RNA, 25 nt, synthetic construct
  misc_feature 1..25: note = Synthetic
  source 1..25: mol_type = other RNA
cgtctgagggatctctagttacctt

SEQ ID NO: 13 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 14 - RNA, 46 nt, synthetic construct; identical to SEQ ID NO: 5
  misc_feature 1..46: note = Synthetic
  source 1..46: mol_type = other RNA

SEQ ID NO: 15 - RNA, 25 nt, synthetic construct
  misc_feature 1..25: note = Synthetic
  source 1..25: mol_type = other RNA
cgtctgagggatctctagttacctt

SEQ ID NO: 16 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 17 - RNA, 46 nt, synthetic construct; identical to SEQ ID NO: 5
  misc_feature 1..46: note = Synthetic
  source 1..46: mol_type = other RNA

SEQ ID NO: 18 - RNA, 25 nt, synthetic construct
  misc_feature 1..25: note = Synthetic
  source 1..25: mol_type = other RNA
cgtctgagggatctctagttacctt

SEQ ID NO: 19 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 20 - RNA, 46 nt, synthetic construct; identical to SEQ ID NO: 5
  misc_feature 1..46: note = Synthetic
  source 1..46: mol_type = other RNA

SEQ ID NO: 21 - RNA, 25 nt, synthetic construct
  misc_feature 1..25: note = Synthetic
  source 1..25: mol_type = other RNA
cgtctgagggatctctagttacctt

SEQ ID NO: 22 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 23 - RNA, 46 nt, synthetic construct; identical to SEQ ID NO: 5
  misc_feature 1..46: note = Synthetic
  source 1..46: mol_type = other RNA

SEQ ID NO: 24 - RNA, 25 nt, synthetic construct
  misc_feature 1..25: note = Synthetic
  source 1..25: mol_type = other RNA
cgtctgagggatctctagttacctt

SEQ ID NO: 25 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 26 - RNA, 46 nt, synthetic construct; identical to SEQ ID NO: 5
  misc_feature 1..46: note = Synthetic
  source 1..46: mol_type = other RNA

SEQ ID NO: 27 - RNA, 25 nt, synthetic construct
  misc_feature 1..25: note = Synthetic
  source 1..25: mol_type = other RNA
cgtctgagggatctctagttacctt

SEQ ID NO: 28 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 29 - RNA, 46 nt, synthetic construct; identical to SEQ ID NO: 5
  misc_feature 1..46: note = Synthetic
  source 1..46: mol_type = other RNA

SEQ ID NO: 30 - RNA, 25 nt, synthetic construct
  misc_feature 1..25: note = Synthetic
  source 1..25: mol_type = other RNA
cgtctgagggatctctagttacctt

SEQ ID NO: 31 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 32 - RNA, 46 nt, synthetic construct; identical to SEQ ID NO: 5
  misc_feature 1..46: note = Synthetic
  source 1..46: mol_type = other RNA

SEQ ID NO: 33 - RNA, 25 nt, synthetic construct
  misc_feature 1..25: note = Synthetic
  source 1..25: mol_type = other RNA
cgtctgagggatctctagttacctt

SEQ ID NO: 34 - DNA, 31 bp, synthetic construct; identical to SEQ ID NO: 4
  misc_feature 1..31: note = Synthetic
  source 1..31: mol_type = other DNA
  misc_feature 1: note = RNA
  misc_feature 2: note = DNA
  misc_feature 3..7: note = RNA
  misc_feature 8: note = DNA
  misc_feature 9..11: note = RNA
  misc_feature 12: note = DNA
  misc_feature 13..15: note = RNA
  misc_feature 16: note = DNA
  misc_feature 17..21: note = RNA
  misc_feature 22: note = DNA
  misc_feature 23..31: note = RNA

SEQ ID NO: 35 - RNA, 46 nt, synthetic construct; identical to SEQ ID NO: 5
  misc_feature 1..46: note = Synthetic
  source 1..46: mol_type = other RNA

SEQ ID NO: 36 - RNA, 25 nt, synthetic construct
  misc_feature 1..25: note = Synthetic
  source 1..25: mol_type = other RNA
cgtctgagggatctctagttacctt

SEQ ID NO: 37 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 38 - RNA, 46 nt, synthetic construct; identical to SEQ ID NO: 8
  misc_feature 1..46: note = Synthetic
  source 1..46: mol_type = other RNA

SEQ ID NO: 39 - RNA, 25 nt, synthetic construct
  misc_feature 1..25: note = Synthetic
  source 1..25: mol_type = other RNA
cgtctgagggatctctagttacctt

SEQ ID NO: 40 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 41 - RNA, 46 nt, synthetic construct; identical to SEQ ID NO: 8
  misc_feature 1..46: note = Synthetic
  source 1..46: mol_type = other RNA

SEQ ID NO: 42 - RNA, 25 nt, synthetic construct
  misc_feature 1..25: note = Synthetic
  source 1..25: mol_type = other RNA
cgtctgagggatctctagttacctt

SEQ ID NO: 43 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 44 - RNA, 46 nt, synthetic construct; identical to SEQ ID NO: 8
  misc_feature 1..46: note = Synthetic
  source 1..46: mol_type = other RNA

SEQ ID NO: 45 - RNA, 25 nt, synthetic construct
  misc_feature 1..25: note = Synthetic
  source 1..25: mol_type = other RNA
cgtctgagggatctctagttacctt

SEQ ID NO: 46 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 47 - RNA, 46 nt, synthetic construct; identical to SEQ ID NO: 8
  misc_feature 1..46: note = Synthetic
  source 1..46: mol_type = other RNA

SEQ ID NO: 48 - RNA, 25 nt, synthetic construct
  misc_feature 1..25: note = Synthetic
  source 1..25: mol_type = other RNA
cgtctgagggatctctagttacctt

SEQ ID NO: 49 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 50 - RNA, 46 nt, synthetic construct; identical to SEQ ID NO: 8
  misc_feature 1..46: note = Synthetic
  source 1..46: mol_type = other RNA

SEQ ID NO: 51 - RNA, 25 nt, synthetic construct
  misc_feature 1..25: note = Synthetic
  source 1..25: mol_type = other RNA
cgtctgagggatctctagttacctt

SEQ ID NO: 52 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 53 - RNA, 46 nt, synthetic construct; identical to SEQ ID NO: 8
  misc_feature 1..46: note = Synthetic
  source 1..46: mol_type = other RNA

SEQ ID NO: 54 - RNA, 25 nt, synthetic construct
  misc_feature 1..25: note = Synthetic
  source 1..25: mol_type = other RNA
cgtctgagggatctctagttacctt

SEQ ID NO: 55 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 56 - RNA, 46 nt, synthetic construct; identical to SEQ ID NO: 8
  misc_feature 1..46: note = Synthetic
  source 1..46: mol_type = other RNA

SEQ ID NO: 57 - RNA, 25 nt, synthetic construct
  misc_feature 1..25: note = Synthetic
  source 1..25: mol_type = other RNA
cgtctgagggatctctagttacctt

SEQ ID NO: 58 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 59 - RNA, 46 nt, synthetic construct; identical to SEQ ID NO: 8
  misc_feature 1..46: note = Synthetic
  source 1..46: mol_type = other RNA

SEQ ID NO: 60 - RNA, 25 nt, synthetic construct
  misc_feature 1..25: note = Synthetic
  source 1..25: mol_type = other RNA
cgtctgagggatctctagttacctt

SEQ ID NO: 61 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 62 - RNA, 46 nt, synthetic construct
  misc_feature 1..46: note = Synthetic
  source 1..46: mol_type = other RNA
gctggtgaagtcgcatcgaaccattagcagaagacgaggtccatga

SEQ ID NO: 63 - DNA, 25 bp, synthetic construct
  misc_feature 1..25: note = Synthetic
  source 1..25: mol_type = other DNA
  misc_feature 1: note = RNA
  misc_feature 2: note = DNA
  misc_feature 3..25: note = RNA
gtcttctgctaatggttcgatgctt

SEQ ID NO: 64 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 65 - RNA, 46 nt, synthetic construct; identical to SEQ ID NO: 62
  misc_feature 1..46: note = Synthetic
  source 1..46: mol_type = other RNA

SEQ ID NO: 66 - DNA, 25 bp, synthetic construct
  misc_feature 1..25: note = Synthetic
  source 1..25: mol_type = other DNA
  misc_feature 1: note = RNA
  misc_feature 2: note = DNA
  misc_feature 3..25: note = RNA
gtcttctgctaatggttcgatgctt

SEQ ID NO: 67 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 68 - RNA, 46 nt, synthetic construct; identical to SEQ ID NO: 62
  misc_feature 1..46: note = Synthetic
  source 1..46: mol_type = other RNA

SEQ ID NO: 69 - DNA, 25 bp, synthetic construct
  misc_feature 1..25: note = Synthetic
  source 1..25: mol_type = other DNA
  misc_feature 1: note = RNA
  misc_feature 2: note = DNA
  misc_feature 3..25: note = RNA
gtcttctgctaatggttcgatgctt

SEQ ID NO: 70 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 71 - RNA, 46 nt, synthetic construct; identical to SEQ ID NO: 62
  misc_feature 1..46: note = Synthetic
  source 1..46: mol_type = other RNA

SEQ ID NO: 72 - DNA, 25 bp, synthetic construct
  misc_feature 1..25: note = Synthetic
  source 1..25: mol_type = other DNA
  misc_feature 1: note = RNA
  misc_feature 2: note = DNA
  misc_feature 3..25: note = RNA
gtcttctgctaatggttcgatgctt

SEQ ID NO: 73 - DNA, 31 bp, synthetic construct; identical to SEQ ID NO: 7
  misc_feature 1..31: note = Synthetic
  source 1..31: mol_type = other DNA
  misc_feature 1..4: note = RNA
  misc_feature 5: note = DNA
  misc_feature 6: note = RNA
  misc_feature 7: note = DNA
  misc_feature 8..21: note = RNA
  misc_feature 22: note = DNA
  misc_feature 23..30: note = RNA
  misc_feature 31: note = DNA

SEQ ID NO: 74 - RNA, 46 nt, synthetic construct; identical to SEQ ID NO: 62
  misc_feature 1..46: note = Synthetic
  source 1..46: mol_type = other RNA

SEQ ID NO: 75 - DNA, 25 bp, synthetic construct
  misc_feature 1..25: note = Synthetic
  source 1..25: mol_type = other DNA
  misc_feature 1: note = RNA
  misc_feature 2: note = DNA
  misc_feature 3..25: note = RNA
gtcttctgctaatggttcgatgctt

SEQ ID NO: 76 - RNA, 27 nt, synthetic construct
  misc_feature 1..27: note = Synthetic
  source 1..27: mol_type = other RNA
cgcgtctgagggatctctagttacctt

SEQ ID NO: 77 - DNA, 27 bp, synthetic construct
  misc_feature 1..27: note = Synthetic
  source 1..27: mol_type = other DNA
  misc_feature 1..21: note = RNA
  misc_feature 22: note = DNA
  misc_feature 23..27: note = RNA
cgcgtctgagggatctctagttacctt

SEQ ID NO: 78 - RNA, 12 nt, synthetic construct
  misc_feature 1..12: note = Synthetic
  source 1..12: mol_type = other RNA
ccctcagacgcg

SEQ ID NO: 79 - RNA, 13 nt, synthetic construct
  misc_feature 1..13: note = Synthetic
  source 1..13: mol_type = other RNA
ggtaactagagat

SEQ ID NO: 80 - RNA, 27 nt, synthetic construct
  misc_feature 1..27: note = Synthetic
  source 1..27: mol_type = other RNA
cgacgagctcatcaggtaactagagat

SEQ ID NO: 81 - RNA, 27 nt, synthetic construct
  misc_feature 1..27: note = Synthetic
  source 1..27: mol_type = other RNA
cgacgaagctcatcggtaactagagat

SEQ ID NO: 82 - RNA, 27 nt, synthetic construct
  misc_feature 1..27: note = Synthetic
  source 1..27: mol_type = other RNA
cgacgaagctcatcggtaactagagat

SEQ ID NO: 83 - RNA, 26 nt, synthetic construct
  misc_feature 1..26: note = Synthetic
  source 1..26: mol_type = other RNA
cgacgagctcatcggtaactagagat

SEQ ID NO: 84 - DNA, 13 bp, synthetic construct
  misc_feature 1..13: note = Synthetic
  source 1..13: mol_type = other DNA
atctctagttacc

SEQ ID NO: 85 - RNA, 67 nt, synthetic construct
  misc_feature 1..67: note = Synthetic
  source 1..67: mol_type = other RNA
gctatggcaggaagaagcggagacagcgacgaagagctcatcagaacagtcggcgcaagcctttttt

SEQ ID NO: 86 - RNA, 67 nt, synthetic construct
  misc_feature 1..67: note = Synthetic
  source 1..67: mol_type = other RNA
gcgaacggcattagcggcacaagagacgacggaagagtcatcagaacagtcggcgcaagcctttttt

SEQ ID NO: 87 - RNA, 67 nt, synthetic construct
  misc_feature 1..67: note = Synthetic
  source 1..67: mol_type = other RNA
gctatggcaggaagaagcggagacagcgcaatccttatcatcagaacagtcggcgcaagcctttttt

SEQ ID NO: 88 - RNA, 67 nt, synthetic construct
  misc_feature 1..67: note = Synthetic
  source 1..67: mol_type = other RNA
gctcactgaggaagaagcggagacagcgacgaagagctcatcagaacagtcggcgcaagcctttttt

SEQ ID NO: 89 - RNA, 69 nt, synthetic construct
  misc_feature 1..69: note = Synthetic
  source 1..69: mol_type = other RNA
gacaggtctcatcgggaggaaatggaggtccatgagctggagaagtccaagcgggcgcaagcctttttt

SEQ ID NO: 90 - RNA, 67 nt, synthetic construct; identical to SEQ ID NO: 85
  misc_feature 1..67: note = Synthetic
  source 1..67: mol_type = other RNA

SEQ ID NO: 91 - RNA, 71 nt, synthetic construct
  misc_feature 1..71: note = Synthetic
  source 1..71: mol_type = other RNA
tcagctccaaggatgacgtgggcaagaacgtccatgagctggagaagtccaagcgggcgcaagcctttttt

SEQ ID NO: 92 - RNA, 70 nt, synthetic construct
  misc_feature 1..70: note = Synthetic
  source 1..70: mol_type = other RNA
gacaggtctcatcgggaggaaatggaggcaagaagacaacaagaccctagtcctgggcgcaagccttttt

SEQ ID NO: 93 - RNA, 29 nt, synthetic construct
  misc_feature 1..29: note = Synthetic
  source 1..29: mol_type = other RNA
cttgcgtctgagggatctctagttacctt

SEQ ID NO: 94 - DNA, 23 bp, synthetic construct
  misc_feature 1..23: note = Synthetic
  source 1..23: mol_type = other DNA
aaggtaactagagatccctcaga

SEQ ID NO: 95 - RNA, 38 nt, synthetic construct
  misc_feature 1..38: note = Synthetic
  source 1..38: mol_type = other RNA
cctcagacgcaagctgatgagctcttcgtcgctgtctc

SEQ ID NO: 96 - RNA, 13 nt, synthetic construct
  misc_feature 1..13: note = Synthetic
  source 1..13: mol_type = other RNA
cctcagacgcaag

SEQ ID NO: 97 - RNA, 30 nt, synthetic construct
  misc_feature 1..30: note = Synthetic
  source 1..30: mol_type = other RNA
cgacgaagagctcatcggtaactagagatc

SEQ ID NO: 98 - DNA, 44 bp, synthetic construct
  misc_feature 1..44: note = Synthetic
  source 1..44: mol_type = other DNA
  misc_feature 1..8: note = RNA
  misc_feature 9: note = DNA
  misc_feature 10..11: note = RNA
  misc_feature 12: note = DNA
  misc_feature 13..44: note = RNA
aaggtccctgatcgacgaagagctcatcagggtaactagagatc

SEQ ID NO: 99 - RNA, 14 nt, synthetic construct
  misc_feature 1..14: note = Synthetic
  source 1..14: mol_type = other RNA
ggtaactagagatc

SEQ ID NO: 100 - RNA, 14 nt, synthetic construct
  misc_feature 1..14: note = Synthetic
  source 1..14: mol_type = other RNA
ggtaactagagatc

SEQ ID NO: 101 - DNA, 40 bp, synthetic construct
  misc_feature 1..40: note = Synthetic
  source 1..40: mol_type = other DNA
aaaaaagcggagacagcgacgaagagctcatcagaaaaaa

SEQ ID NO: 102 - RNA, 27 nt, synthetic construct
  misc_feature 1..27: note = Synthetic
  source 1..27: mol_type = other RNA
cctcagacgcaagggtaactagagatc

SEQ ID NO: 103 - DNA, 32 bp, synthetic construct
  misc_feature 1..32: note = Synthetic
  source 1..32: mol_type = other DNA
tcgagtctggtaactagagatccctcagaccc

SEQ ID NO: 104 - DNA, 32 bp, synthetic construct
  misc_feature 1..32: note = Synthetic
  source 1..32: mol_type = other DNA
ggccgggtctgagggatctctagttaccagac

SEQ ID NO: 105 - DNA, 28 bp, synthetic construct
  misc_feature 1..28: note = Synthetic
  source 1..28: mol_type = other DNA
tcgagctgcatcgaaccattagcagaaa

SEQ ID NO: 106 - DNA, 28 bp, synthetic construct
  misc_feature 1..28: note = Synthetic
  source 1..28: mol_type = other DNA
ggcctttctgctaatggttcgatgcagc

SEQ ID NO: 107 - DNA, 10 bp, synthetic construct
  misc_feature 1..10: note = Synthetic
  source 1..10: mol_type = other DNA
ggcgcaagcc

SEQ ID NO: 108 - RNA, 76 nt, synthetic construct
  misc_feature 1..76: note = Synthetic
  source 1..76: mol_type = other RNA
gcccggatagctcggtcggtggagcatcagacttttaatctgagggtccagggttcgagtccctgttcgtgcacca

SEQ ID NO: 109 - DNA, 22 bp, synthetic construct
  misc_feature 1..22: note = Synthetic
  source 1..22: mol_type = other DNA
ctggaccctcagattaaaagtc

SEQ ID NO: 110 - DNA, 27 bp, synthetic construct
  misc_feature 1..27: note = Synthetic
  source 1..27: mol_type = other DNA
ctccatttcctcccgatgagacctgtc

SEQ ID NO: 111 - DNA, 26 bp, synthetic construct
  misc_feature 1..26: note = Synthetic
  source 1..26: mol_type = other DNA
cgcttggacttctccagctcatggac

SEQ ID NO: 112 - DNA, 24 bp, synthetic construct
  misc_feature 1..24: note = Synthetic
  source 1..24: mol_type = other DNA
aaaggtaactagagatccctcaga

SEQ ID NO: 113 - RNA, 40 nt, synthetic construct
  misc_feature 1..40: note = Synthetic
  source 1..40: mol_type = other RNA
cgacgaagctcatcaggtaactagagatccctcagacgcg

(19) United States
(12) Patent Application Publication
(10) Pub. No.: US 2024/0401043 A1
(43) Pub. Date: Dec. 5, 2024

(54) PROGRAMMABLE CONDITIONAL SIRNAS AND USES THEREOF

(71) Applicants: CITY OF HOPE, Duarte, CA (US); CALIFORNIA INSTITUTE OF TECHNOLOGY

(72) Inventors: Si-ping HAN, Duarte, CA (US); William A. GODDARD, III, Pasadena, CA (US); Marwa Ben HAJ SALAH, Duarte, CA (US); Lisa SCHERER, Duarte, CA (US); John J. ROSSI, Duarte, CA (US)

(21) Appl. No.: 18/667,951

(22) Filed: May 17, 2024

Related U.S. Application Data
(63) Continuation of application No. 17/172,461, filed on Feb. 10, 2021, now Pat. No. 11,999,954.
Application No. 17/172,461 is a continuation of application No. PCT/US19/46075, filed on Aug. 10, 2019.
(60) Provisional application No. 62/717,686, filed on Aug. 10, 2018.
(60) Provisional application No. 62/811,183, filed on Feb. 27, 2019.

(51) Int. Cl. C12N 15/113

(52) U.S. Cl. CPC C12N 15/113; C12N 2310/141

(57) ABSTRACT

Disclosed herein are programmable, conditionally activated small interfering RNA constructs (Cond-siRNAs) and methods of making and using the same as therapeutic agents. The Cond-siRNA comprises a sensor strand, a core strand, and a guide strand, which crossover to form a sensor duplex and a RNAi duplex attached to each other to form a single structure. Upon binding an input strand to the sensor strand, the Cond-siRNA is activated and releases RNAi targeting a desired gene.

### PRIORITY CLAIM

This application is a continuation of U.S. application Ser. No. 17/172,461 filed Feb. 10, 2021, which is a continuation of International Patent Application No. PCT/US2019/046075, filed Aug. 10, 2019, which claims priority to U.S. Provisional Application Nos. 62/717,686, filed on Aug. 10, 2018, and 62/811,183, filed on Feb. 27, 2019, the contents of which are incorporated by reference herein in their entireties, including drawings.

### STATEMENT OF GOVERNMENT INTEREST

This invention was made with government support under grant number A1029329, awarded by the National Institutes of Health and grant numbers 1332411 and 1120890, awarded by the National Science Foundation. The government has certain rights in the invention.

### SEQUENCE LISTING

The present application is being filed along with a Sequence Listing in electronic format. The Sequence Listing is provided as a file entitled 75EN-329804-US2_SequenceListing, created May 7, 2024, which is 237,637 bytes in size. The information in the electronic format of the Sequence Listing is incorporated herein by reference in its entirety.

### BACKGROUND

A longstanding goal for nucleic acid nanotechnology¹⁻³ and biomolecular computing⁴ is the development of conditionally activated oligonucleotide therapeutics that can detect and respond to cellular expression of specific genes^{3,4}. Nucleic acid switches based on toehold mediated strand displacement^{5,6} have executed logic operations and detected RNA transcripts in both bacteria⁷ and mammalian cells^{3,8}, but the conditional activation of oligonucleotide drugs by RNA transcripts in mammalian cells has not been convincingly demonstrated. Significant challenges include poorly suppressed background drug activity, weak ON state drug potency, input and output sequence overlap, high design complexity, short device lifetimes (<24 hours) and high required device concentrations (>10 nM).

Over the past decade, synthetic RNAi triggers such as small interfering RNAs (siRNAs)¹⁰ have become ubiquitous tools in biological research, and extensive basic and clinical development efforts have recently culminated in the FDA approval of ONPATTRO, the first RNAi drug¹¹. Despite a burgeoning drug development pipeline and an extensive compendium of excipients targeting ligands and delivery techniques⁹, the difficulty of delivering RNAi agents to specific populations of disease related cells continues to limit the potential of RNAi therapy. Repeated attempts over the past fifteen years to develop programmable, conditionally activated RNAi agents based on strand displacement switches¹²⁻¹⁵ have not convincingly demonstrated the intended effects, despite notable progress^{3,8,16-19}. Thus, a new conditionally activated siRNA (Cond-siRNA) is provided herein to overcome the problems in the art.

### SUMMARY

In one aspect, this disclosure relates to a programmable, conditionally activated small interfering RNA construct (Cond-siRNA) in an OFF state, the construct comprising a sensor strand, a core strand, and a guide strand, wherein the sensor strand and the core strand bind complementarily to form a sensor duplex, the guide strand and the core strand bind complementarily to form a RNAi duplex, and the sensor duplex and the RNAi duplex are attached to each other to form a single structure. In some embodiments, the sensor duplex and the RNAi duplex are attached to each other via the core strand. In some embodiments, the sensor strand complementarily binds to a fragment on the 5′ and a fragment on the 3′ of the core strand to form the sensor duplex, and the guide strand complementarily binds to a fragment in the middle of the core strand to form the RNAi duplex and the fragment in the middle does not comprise any 5′ or 3′ sequence of the core strand such that the sensor duplex and RNAi duplex are attached to each other via two different fragments of the core strand. In some embodiments, the sensor domain of the Cond-siRNA construct comprises a sensor duplex formed by complimentary binding of the sensor strand and 3′ and 5′ fragments of the core strand, and a sensor overhang that does not pair up with the core strand. The sensor overhang is either at the 3′ end or at the 5′ end of the sensor strand. In some embodiments, the sensor strand, core strand, and guide strand form the single construct via self-assembling upon contact of each other. In some embodiments, the sensor duplex comprises 15, 16, 17, 18, 19, 20, 21, 22, 23, 24, 25, 26, 27, 28, 29, or 30 base pairs. In some embodiments, the RNAi duplex comprises 15, 16, 17, 18, 19, 20, 21, 22, 23, 24, 25, 26, 27, 28, 29, or 30 base pairs.

In some embodiments, the Cond-siRNA is chemically modified to further improve the OFF-state stability and/or dissociation efficiency when turned on upon contact with an input strand. For example, the bases of the sensor strand in the duplex region is modified by LNA modification, 2′-O-methyl modification, or both but not by phosphorothioate (PS) modification; either or both termini of the core strand are modified with PS modification, 2′-O-methyl modification, or both; or the single strand overhang of the sensor strand is modified by LNA modification, 2′-O-methyl modification, PS modification, or any combination thereof. In some embodiments, the sensor domain and the RNAi domain are modified by different chemical modifications.

The OFF-state Cond-siRNA described above is activated or turned on by contacting the OFF-state Cond-siRNA with an input strand, wherein one end of the input strand forms a toehold with the sensor strand overhang to induce displacement of the sensor strand from the core strand via complementary binding of the input strand and the sensor strand to form a waste duplex, whereby the RNAi duplex is completely disassociated from the construct.

### BRIEF DESCRIPTION OF THE DRAWINGS

This application contains at least one drawing executed in color. Copies of this application with color drawing(s) will be provided by the Office upon request and payment of the necessary fees.

FIGS. 1a-d show conceptual design, operation, and molecular dynamics simulation of Cond-siRNAs. FIG. 1a shows the conceptual secondary and tertiary structure of the Cond-siRNA. Docking of the RNAi duplex to an x-ray crystal structure of Giardia Dicer shows massive steric clashes between the sensor duplex and Dicer. FIG. 1b shows RNAi activation via strand displacement. When a complementary input RNA meets the Cond-siRNA (I), the input forms a toehold with a 3′ or 5′ single stranded overhang on the sensor strand (II), leading to strand displacement (III). Displacement results in separation of the sensor strand from the RNAi duplex (IV). Cellular nucleases remove the core strand overhangs on the RNAi duplex (V), leaving an active RNAi trigger for Dicer processing (VI). FIGS. 1c and 1d show the Molecular Dynamics (MD) optimized models of constructs I.1 and III.1. Green block arrows denote direction of strand displacement from the 3′ and 5′ ends of the sensor strand.

FIG. 2 (SEQ ID NOS: 77 and 113) shows intracellular degradation of chemically modified single stranded overhangs. Northern blot shows stepwise degradation of phosphorothioate (PS) protected 5′ overhangs on test constructs. Test constructs (Dicer substrates with segmented passenger strand and various 5′ overhangs) were transfected into HCT116 cells for 24 hours. The total RNA was extracted and analyzed via Northern blot. Two sets of similar samples were assayed, showing similar results at differing loading concentrations.

FIGS. 3a-3f show sequence diagrams and map of optimization regions of prototype constructs (FIG. 3a) (SEQ ID NOS: 1-12), construct II variants (FIGS. 3b, 3c) (SEQ ID NOS: 13-36), construct IlI variants (FIGS. 3d, 3e) (SEQ ID NOS: 37-60), and construct IV variants (FIG. 3f) (SEQ ID NOS: 61-69).

FIGS. 4a-4b show Northern blot of RNA inputs in HCT 116 cells. Northern blot assay probing tat/rev and AML input RNA recovered from HCT 116 cells after 48 hours. FIG. 4a shows tat/rev RNA transcripts probed with mutant tRNALys3 matching their common leader sequence. Lanes: (L) Ambion decade marker; (0) negative control with RNA from mock transfection; (1) fully matching input RNA; (2) 5′ mismatched input; (3) fully mismatched input; (4) duplex mismatched activator (not used); (5) 3′ mismatched activator. Expected size of the input RNA was 145-150 nt. FIG. 4b shows CBFB-MYH11 RNA transcripts. Lanes: (0) mock transfection; (1) tat/rev full match input (for comparison); (2) CBFB-MYH11 fusion; (3) MYH11 parental; (4) MYH11 parental. Successive panels show the same samples probed with mutant tRNALys3 probe, MYH11 probe, and CBFB probe. Expression levels of the input RNA were comparable across all cohorts.

FIGS. 5a-5k show assembly, strand displacement, RNAi activity and RNAi activation of construct I, II, and III variants. FIGS. 5a, 5d show non-denaturing PAGE of construct I and II assembly and isothermal strand displacement in 1×PBS buffer at 37° C. Constructs I and II are disassembled by their respective inputs and unaffected by mismatched inputs. Control lanes are: I=input, C=construct, P=RNAi duplex, W=waste duplex. FIGS. 5b, 5e show RNAi activities of constructs in the presence of mismatched inputs or null inputs and ON state constructs in the presence of mismatched inputs. FIGS. 5c, 5f show RNAi activity of OFF state constructs in cells expressing indicated inputs. g and h, OFF and ON state RNAi activity of construct II variants in cells expressing mismatched inputs. FIGS. 5i, 5j show RNAi activity of OFF state construct II and III variants with different core strand modifications in cells expressing either mismatched or matching inputs. FIG. 5k shows RNAi activity of construct III variants with different sensor strand modifications in cells with matching or mismatched inputs.

FIG. 6 shows Dicer processing of pre-activated Cond-siRNA constructs. Northern blot assay probing Cond-siRNA guide strands recovered from HCT 116 cells after 48 hours. Lanes are as follows: (L) Ambion decade marker; (0) RNA from cells with mock transfection; (1) and (2) guide strands from a third prototype Cond-siRNA not reported in this paper; (3) and (4) OFF and ON states of prototype the HIV construct; (5) and (6) OFF and ON states of the AML construct. Arrow marks position of Dicer cleaved guide strand. Dicer cleavage products (˜21 nt guide strand fragment) were detected in RNA material extracted from cells transfected with ON state Cond-siRNAs, but not from cells transfected with OFF state Cond-siRNAs.

FIG. 7 shows that the base-pair parameters of 23 base-pairs in each helix was measured every 5 ps using x3DNA⁴³ and plotted as heat maps. Base-pairs were numbered from top (1) to bottom (23) in the orientation shown below. Base-pair parameter definitions are illustrated in the website x3dna.org/articles/seeing-is-understanding-as-well-as-believing.

FIGS. 8a-8l show base-pair parameters from Molecular Dynamics simulations of sensor and siRNA duplexes over 5 nanoseconds. Shear (8g), buckle (8k), stretch (8i), propeller (8f), stagger (8h) and opening (8l) parameters are defined for base-pair 1-23. Shift (8e), tilt (8j), slide (8d), roll (8c), rise (8b), and twist (8a) parameters are measured for base-pairs 1-22 versus the succeeding base-pair. The heat scale for each measurement is centered on the average values measured over 5 ns control MD trajectories for four normal unmodified RNA duplexes with identical base sequences as the siRNA and sensor duplexes of the HIV and AML constructs. The heat scales span three standard deviations as measured from the same control trajectories. We observed significant deviations from normal RNA values for twist, rise, roll, slide, shift, and propeller measures (8a-8f). In these plots, construct 1 is the HIV construct, construct 2 is the AML construct.

FIGS. 9a-9c (SEQ ID NOS: 70-72) show RNAi activity, sensor design, and sequence of CBFβ-MYH11 sensing, MCL-1 targeted Cond-siRNA. FIG. 9a shows RNAi activity of construct IV variants in cells expressing irrelevant, fusion activator, MYH11, or CBFB transcripts. FIG. 9b shows sensor strand binding position on each transcript. FIG. 9c shows sequence map of IV.3. Yellow highlights show areas (regions A, B, C and D) with optimized chemical modification motifs.

FIGS. 10a-10b show the relative RNAi activity for different RNA inputs for constructs I.1 (FIG. 10a) and III.1 (FIG. 10b). The target expression data from FIGS. 5c and 5f were renormalized against expression levels for cells transfected with Cond-siRNA but expressing fully mismatched input. Results show that target expression levels in cells expressing matching inputs were lower than those in cells expressing mismatched inputs. FIG. 10a: For construct I.1, cells expressing 5′ mismatched and fully matched inputs had decreased target expression. FIG. 10b: For III.1, cells expressing the fusion input had decreased target expression.

FIG. 11 shows the Northern blot of various prior generation RNAi trigger designs transfected at 1 nM concentration into HCT116 cells for 24 hours. The secondary structure of the triggers is diagramed. Green bubbles indicated 2′-O-methyl RNA bases. Blue bubbles indicate DNA. White bubbles indicate RNA. Black arrow indicates Dicer product. Results show that duplex RNA with adjacent 2′-O-methyl modified duplexes had reduced Dicer products.

FIGS. 12A-12C (SEQ ID NOS: 73-75) illustrate an example of the Cond-siRNA disclosed herein. FIG. 12A illustrates the structure of the construct, where the sensor domain and the siRNA domain are shaded in gray and the sensor duplex is shown in the box. FIG. 12B illustrates the cores strand regions I, II, and III, as well as the exonuclease blocking regions I and II in the core strand. FIG. 12C illustrates the sensor strand regions I, II, and III.

### DETAILED DESCRIPTION

Disclosed herein are approaches for developing programmable, conditionally activated small interfering RNAs (Cond-siRNAs). These simple riboswitches can maintain their integrity over days in the mammalian cytosol and detect cellular RNA transcripts from specific input genes via toehold mediated strand displacement. Upon input detection, the Cond-siRNAs can release potent RNAi triggers⁹ silencing specified target genes with completely independent sequences from the input. As demonstrated in the working examples, the switching activity of dozens of Cond-siRNA variants were tested in human adherent cells to identify necessary and sufficient chemical modification motifs that allow good device performance over diverse input: output combinations. Some optimized Cond-siRNAs achieved more than 90% silencing of target genes (protein expression versus baseline) in cells expressing sequence-matched RNA transcripts, and strongly suppressed background RNAi activity (<25% knockdown) in cells expressing mismatched inputs. Thus, provided herein is a method of substantively improving the performance of strand displacement switches in live mammalian cells. The Cond-siRNA technology provides a practical and versatile platform for gene expression activated RNAi smart drugs.

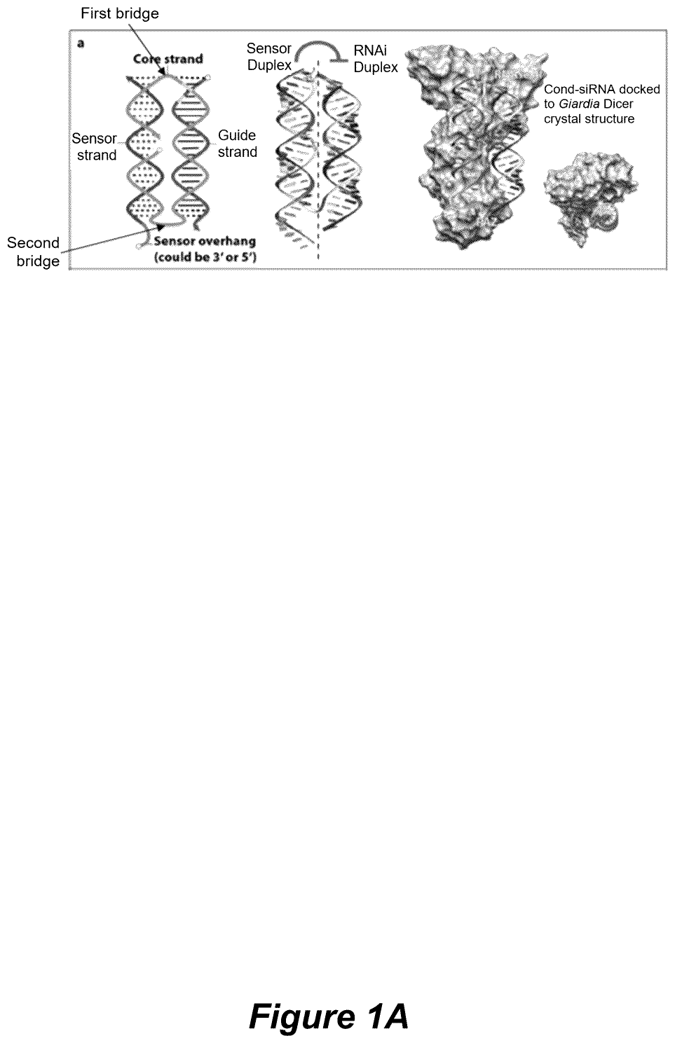

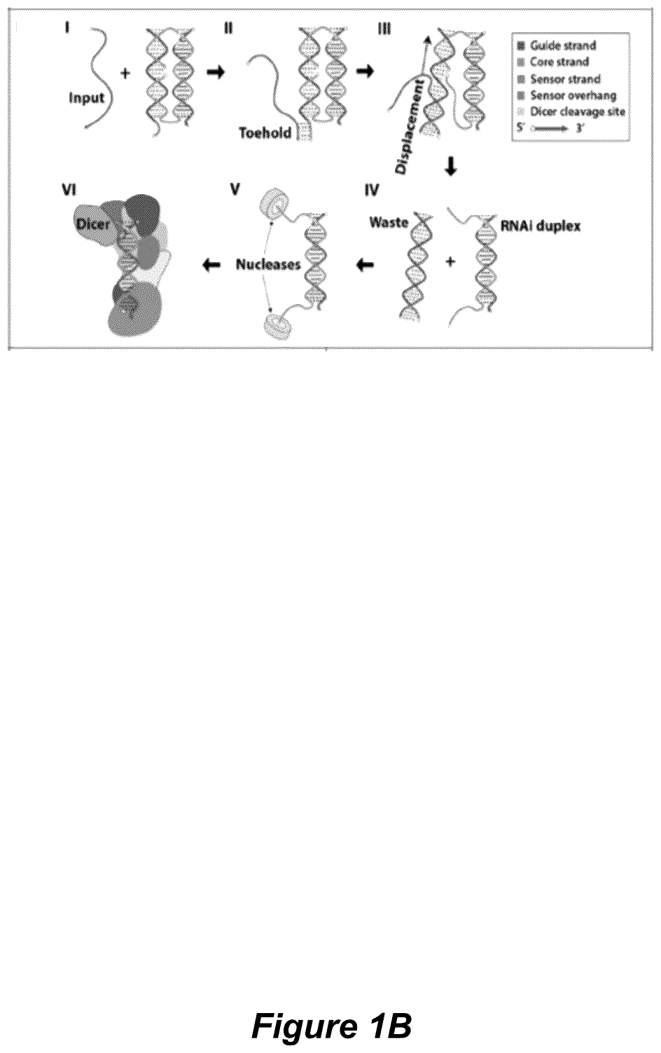

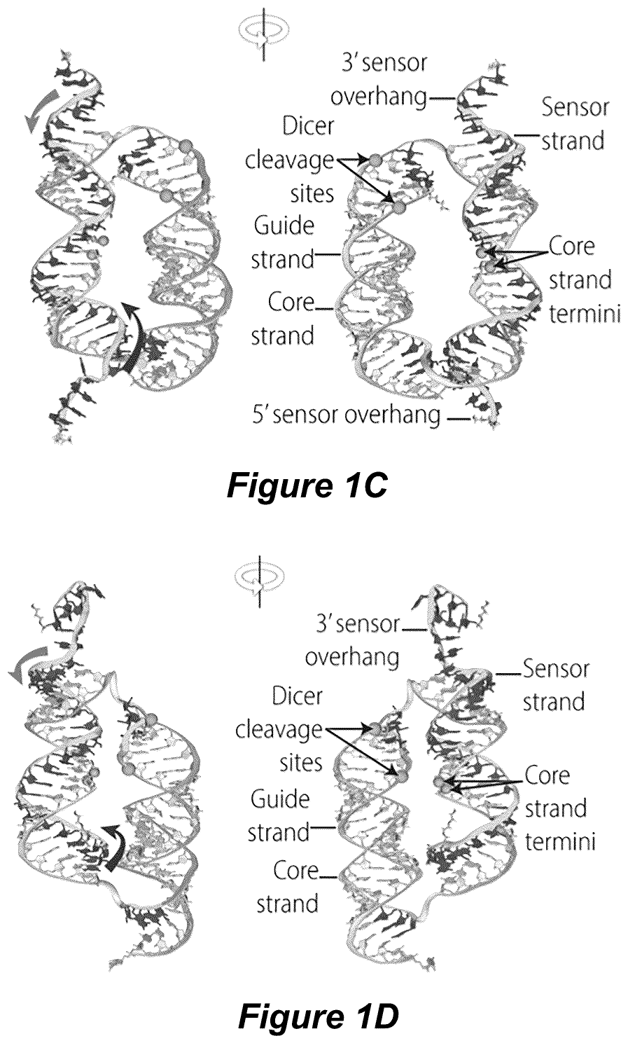

The Cond-siRNA constructs disclosed herein comprises two domains, the sensor domain and the RNAi domain, linked to each other by two fragments of the core strand. This structure is obtained by complementary binding of the sensor strand and the 3′ fragment and the 5′ fragment of the core strand to form a sensor duplex, and the complementary binding of the guide strand and a middle fragment of the core strand to form a RNAi duplex. As illustrated in FIG. 1, not the entire sequence of the core strand complementarily binds to the sensor strand or the guide strand. Rather, a first fragment in the core strand between the 3′ fragment binding to the sensor strand and the middle fragment binding to the guide strand remains single-stranded to form a first “bridge” connecting a first end of the sensor domain to a first end of the RNAi domain. Likewise, a second fragment in the core strand between the 5′ fragment binding to the sensor strand and the middle fragment binding to the guide strand remains single-stranded to form a second “bridge” connecting a second end of the sensor domain to a second end of the RNAi domain.

As used herein, the phrase “complementary binding” or “bind complementarily” means that two single strands are base paired to each other to form a double-stranded duplex. However, a certain percentage of mismatches between the two single strands are allowed as long as a stable double stranded duplex can be formed. For example, in some embodiments, the sensor duplex or the RNAi duplex has about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% mismatches.

### A. Input Strand

Input strands for Cond-siRNAs are “triggers” that switch on the Cond-siRNAs and are usually cellular RNA transcripts that are present at relatively high expression levels in a set of targeted cells (e.g. cancer cells) and at a relatively low level of expression in a set of non-targeted cells (e.g. normal cells). Based on the design of the disclosed Cond-siRNA constructs, only in the targeted cells, the RNAi is turned on; while in the non-targeted cells, the RNAi remains in OFF state. In the targeted cells, the input strand is expressed at a level at least 2-fold, at least 5-fold, at least 10-fold, at least 20-fold, at least 30-fold, at least 40-fold, at least 50-fold, at least 60-fold, at least 70-fold, at least 80-fold, at least 90-fold, or at least 100-fold higher than in the non-targeted cells. Alternatively, in the targeted cells, the input strand is expressed at a level of at least 50, at least 100, at least 200, at least 300, at least 400, at least 500, at least 600, at least 700, at least 800, at least 900, at least 1000 transcripts; and in the non-targeted cells, the input strand is expressed at a level of less than 50, less than 40, less than 30, less than 20, or less than 10 transcripts. Preferably, the non-targeted cells have no detectable expression of the input strand.

The input strand includes an mRNA, an miRNA, or a non-coding RNA such as a long non-coding RNA, an RNA fragment, or an RNA transcript of a virus. For therapeutic uses, the input RNA is usually an RNA transcript that is expressed in a set of cells that are causing the progression of a disease and are therefore targeted for RNAi therapy. The non-targeted cells are usually a set of cells where silencing of the RNAi target can cause side effects that are not beneficial for therapy. For treating a disease or a condition where the input RNA is overexpressed in targeted cells, the Cond-RNAi is designed such that the sensor strand has a sequence complementary to the input RNA sequence. Upon administration of the Cond-RNAi, the binding of sensor strand to the input RNA induces the dissociation of the RNAi duplex from the sensor duplex in targeted cells thereby to activate the RNAi targeting the disease or condition. In non-targeted cells the Cond-RNAi remains inactive.

### B. Construct Design

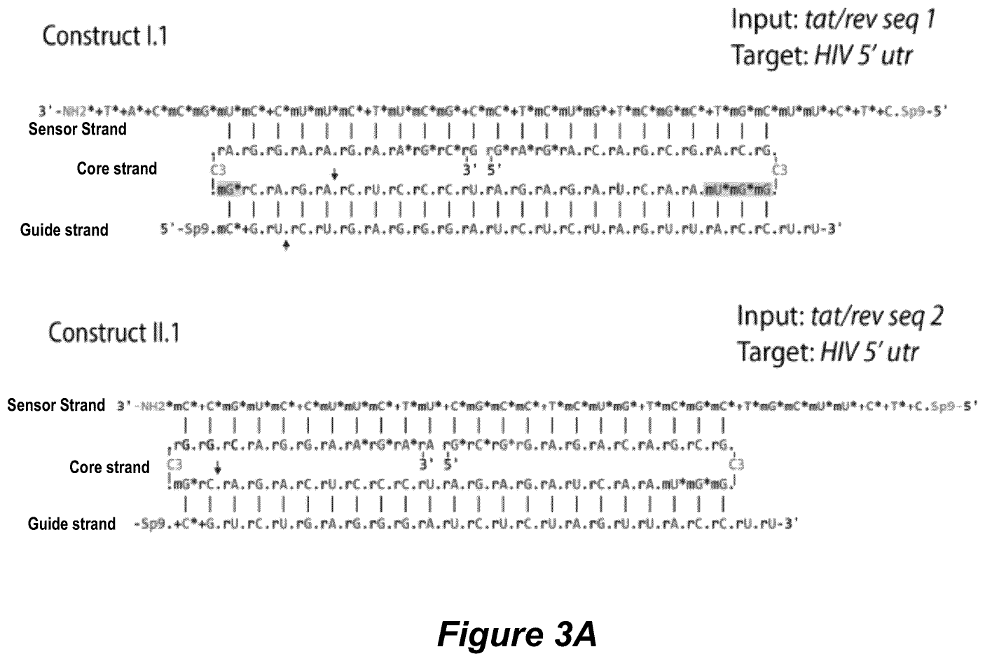

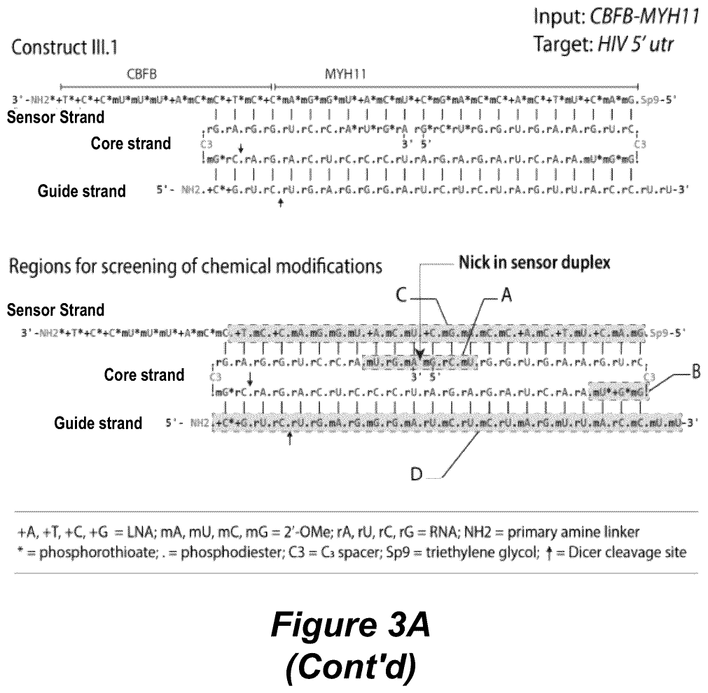

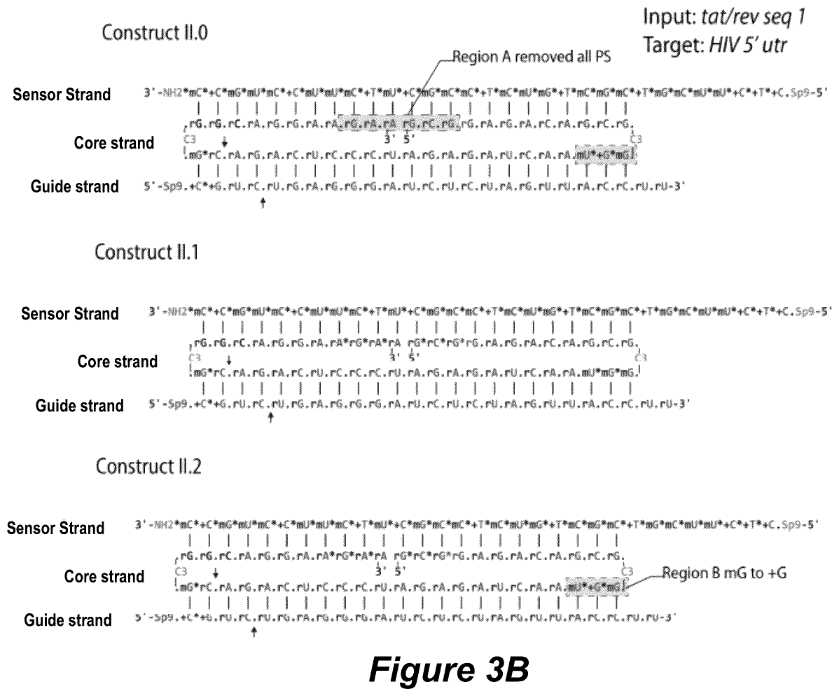

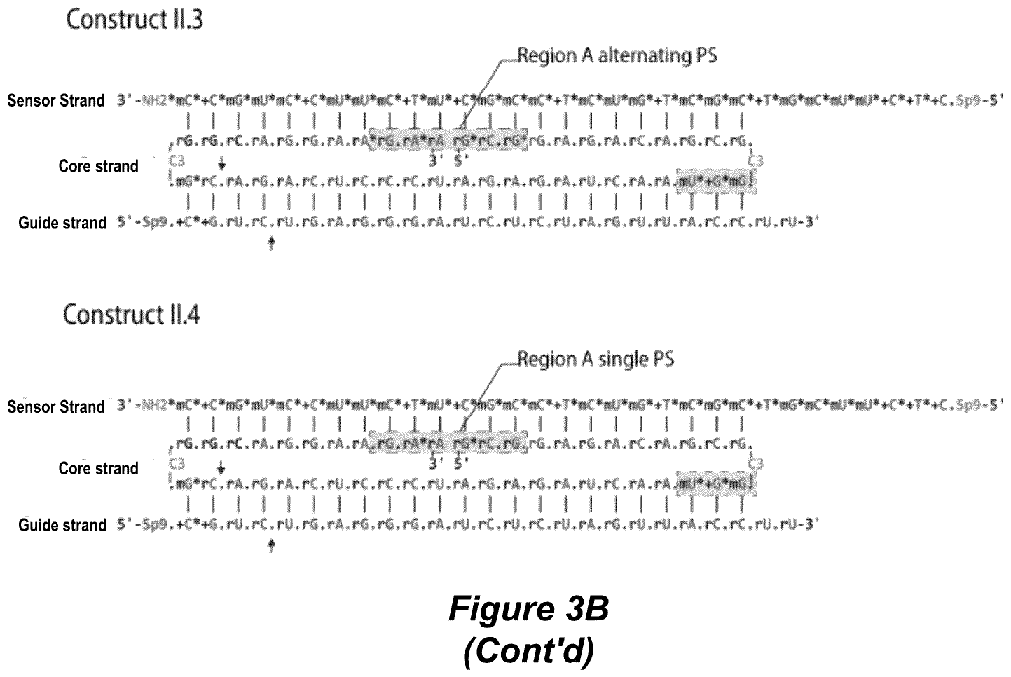

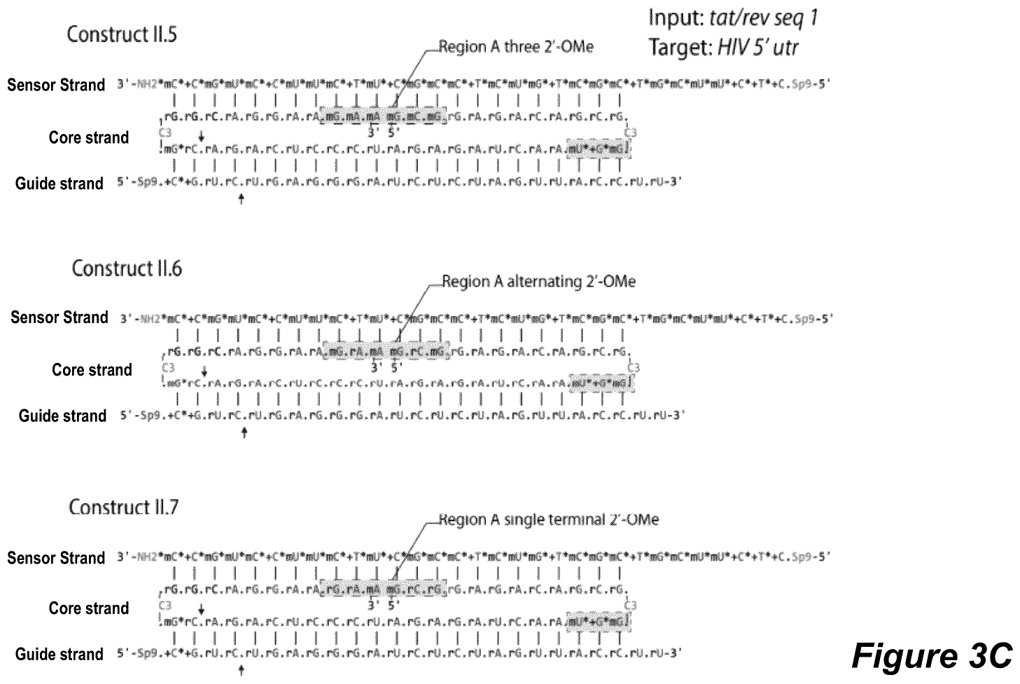

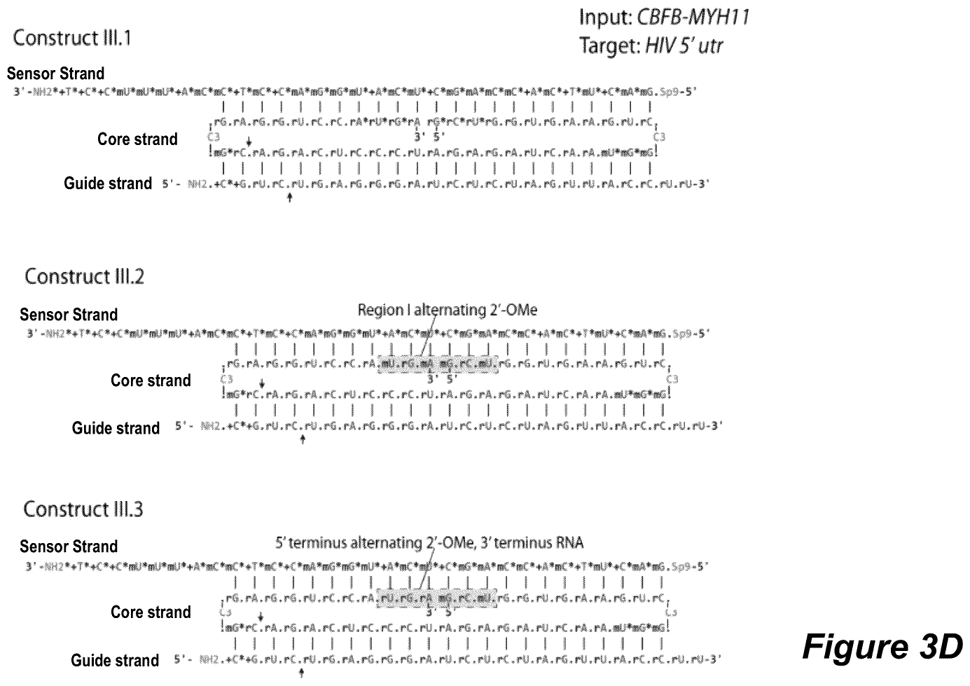

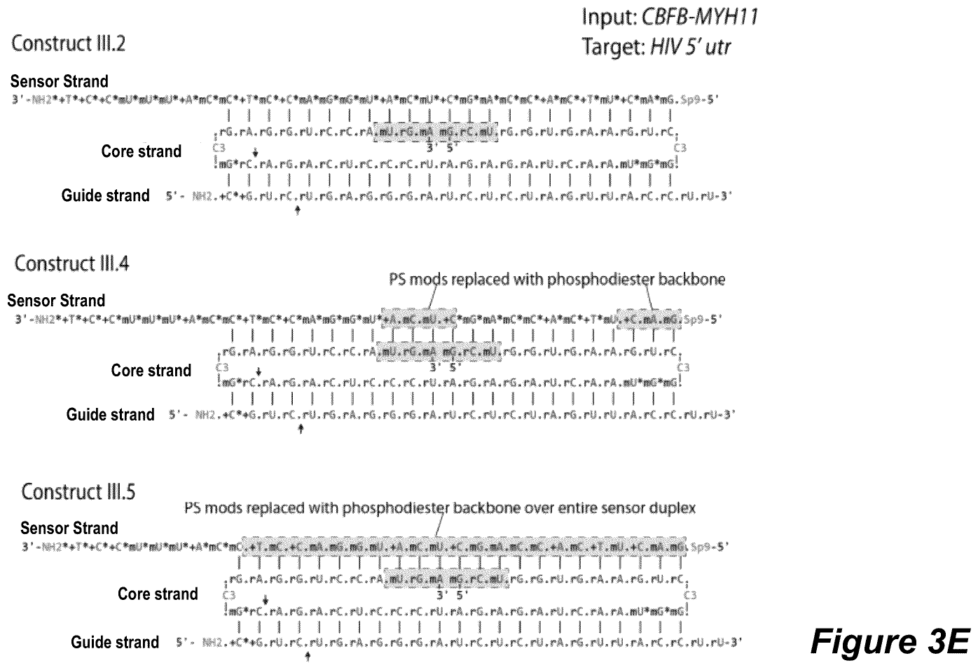

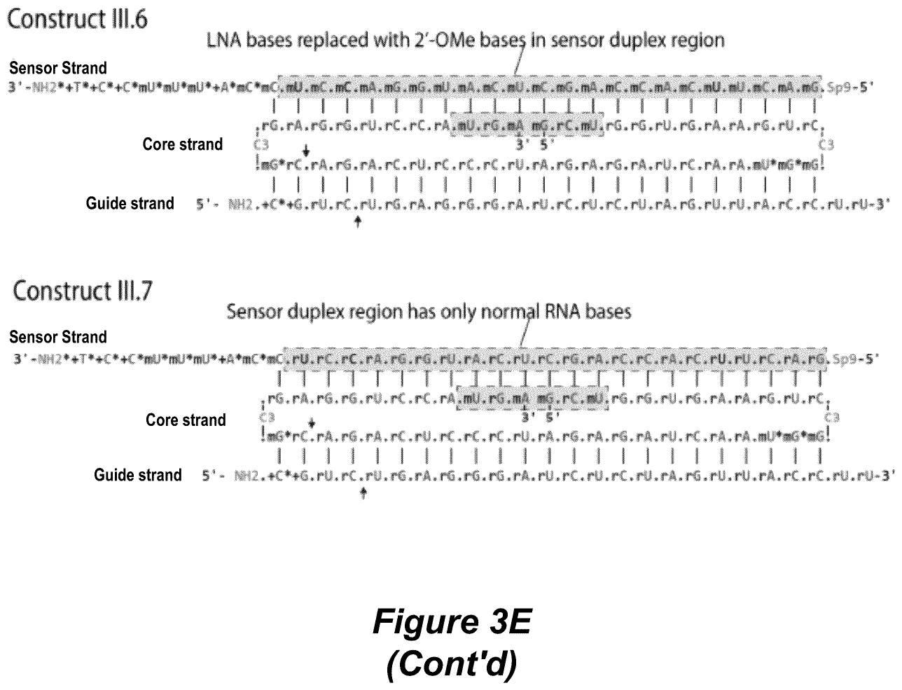

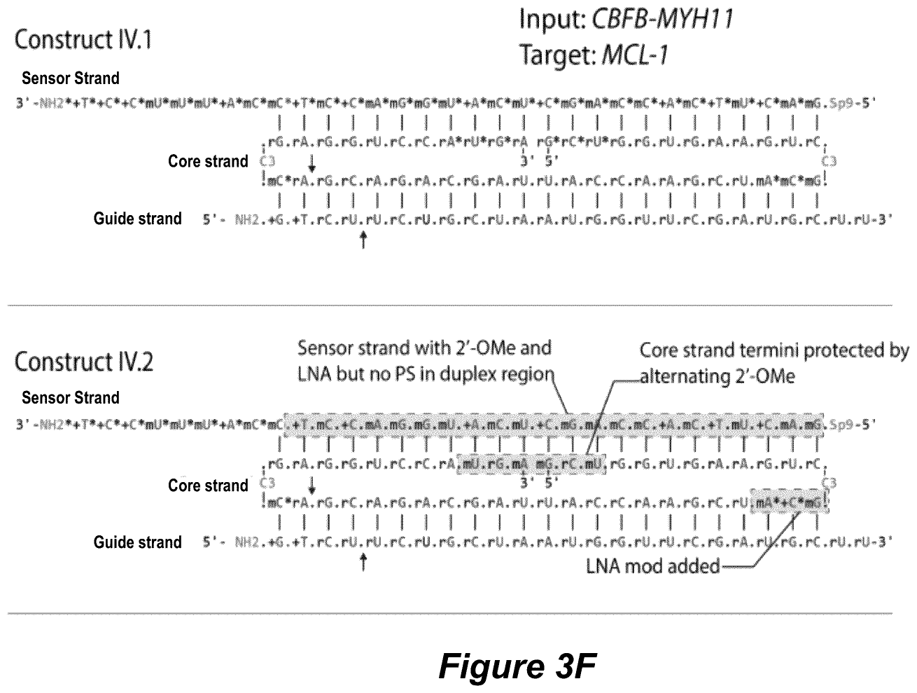

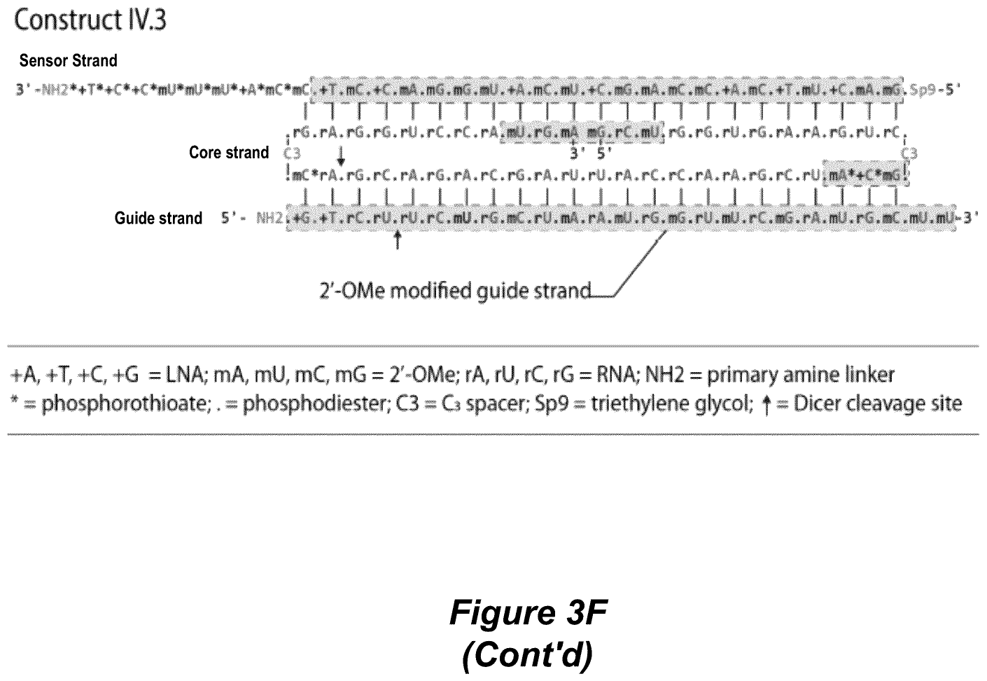

As disclosed herein, Cond-siRNA constructs are designed for specific pairing of inputs and targets using an iterative protocol, exemplified below:
1. Obtain a guide strand sequence for the RNAi domain from previously validated siRNAs, literature sources, or siRNA design tools. In some embodiments, the guide strand has a size of 21 nt.
2. Create a Dicer substrate from the chosen guide strand by adding four G/C rich bases to the 5′ of the guide strand. In some embodiments, the Dicer substrate has a size of 23 bp. Use Nupack (RNA strand, Mathews et al 1999 parameters, some dangle treatment) to check that the RNAi duplex forms with >95% probability at 1 nM concentration of guide (antisense) and sense strands.
3. From the sequence of the input biomarker, generate a list of all possible sensor segments which are antisense to the input strand. In some embodiments, the sensor segments are 31-33 nt in size. For the CBFB-MYH11 fusion sequence, only sensor segments that approximately meet parameters illustrated in FIG. 3b were considered.
4. Rank sensor sequences for uniqueness in the transcriptome of the target animal using NCBI BLAST. For human cancer cell lines, sequences were checked against human transcript and genomic collection using the BLASTn algorithm. Where possible, eliminate sensor segments that have more than 17 bases of sequence complementarity AND complete overhang complementarity to known or predicted RNA transcripts.
5. Starting with the most unique sensor segments, construct core strand sequences in accordance with desired structural parameters for the Cond-siRNA. Core strands have sequences of the form 5′-B-C₃-P-C₃-A-3′ where A and B are complementary to the 5′ and 3′ ends of the sensor strand's putative duplex domain, P is complementary to the putative guide strand, and C₃ are C₃ linkers.
6. Use Nupack to rank the thermodynamic stability of the duplexes formed between sensor strand segments and their corresponding 5′ and 3′ core strand overhangs. Use RNA strand, Mathews et al 1999 parameters, with some dangle treatment. Ideally, >95% of strands should be base-paired at 1 nM strand concentration. Also verify that the core strand does not have heavy internal secondary structure.
7. Choose the best constructs (guide, core, and sensor sequences) generated in steps 1-6.
8. Add chemical modifications disclosed herein.
9. Use oligonucleotide design tools such as “LNA Oligo Tm Prediction” (qiagen.com/us/service-and-support/learning-hub/technologies-and-research-topics/lna/custom-lna-design-and-applications/lna-design-tools-calculators/lna-oligo-tm-prediction/) or “LNA Oligo Optimizer” (qiagen.com/us/service-and-support/learning-hub/technologies-and-research-topics/lna/custom-lna-design-and-applications/lna-design-tools-calculators/lna-oligo-optimizer/) to optimize the placement of LNA modifications. LNA modifications are added to the sensor strand approximately 1 LNA per every 3 to 4 bases. Use the “LNA Oligo Optimizer” tool to check that the LNA pattern used does not lead to secondary structure or self-pairing interactions with scores higher than 60. Minimize self-complementarity and self-pairing scores to the extent possible. Use the “LNA Tm Prediction” tool to check the Tm of the LNA modified oligo when paired with RNA. Choose a placement that maximizes Tm while avoiding self-pairing interactions and secondary structure scores higher than 60 using the “LNA Oligo Optimizer” tool.

### C. Design Features of Core Strand

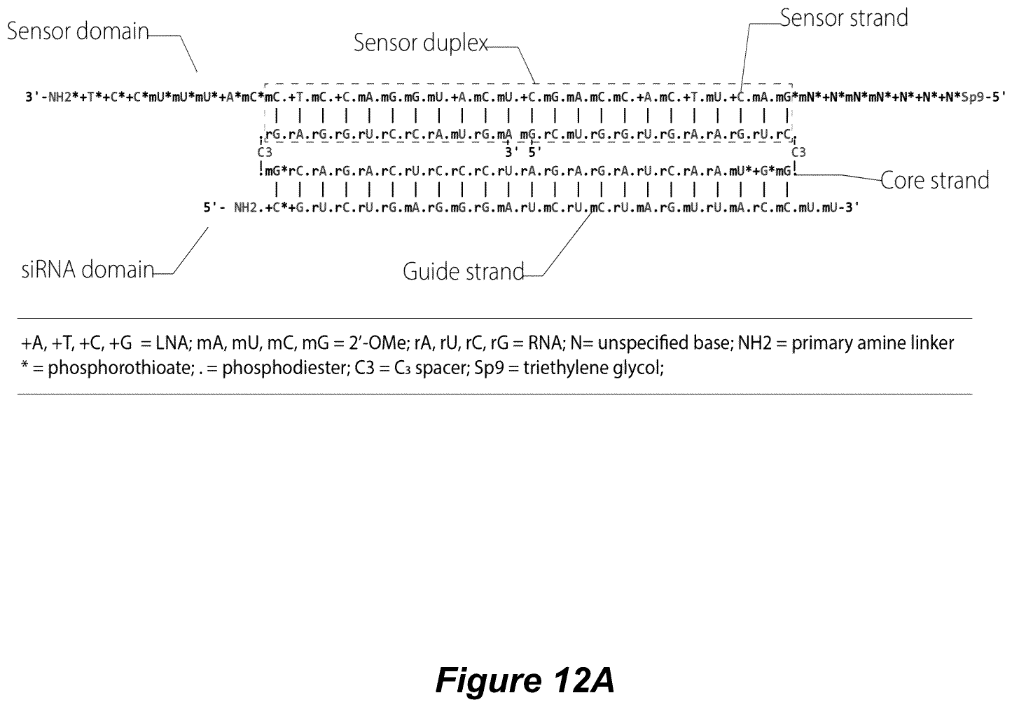

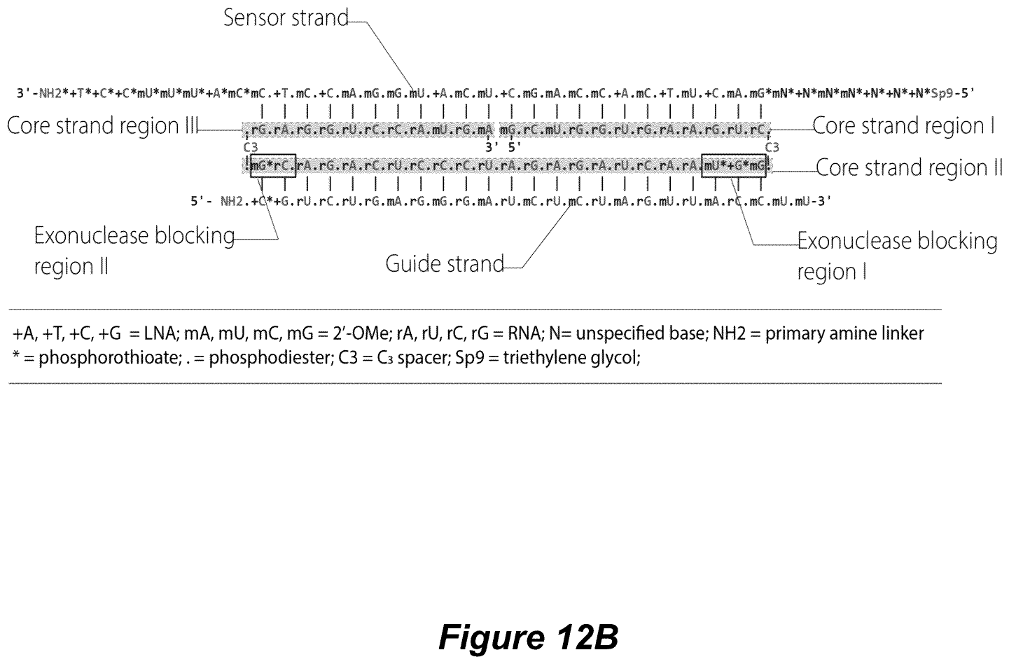

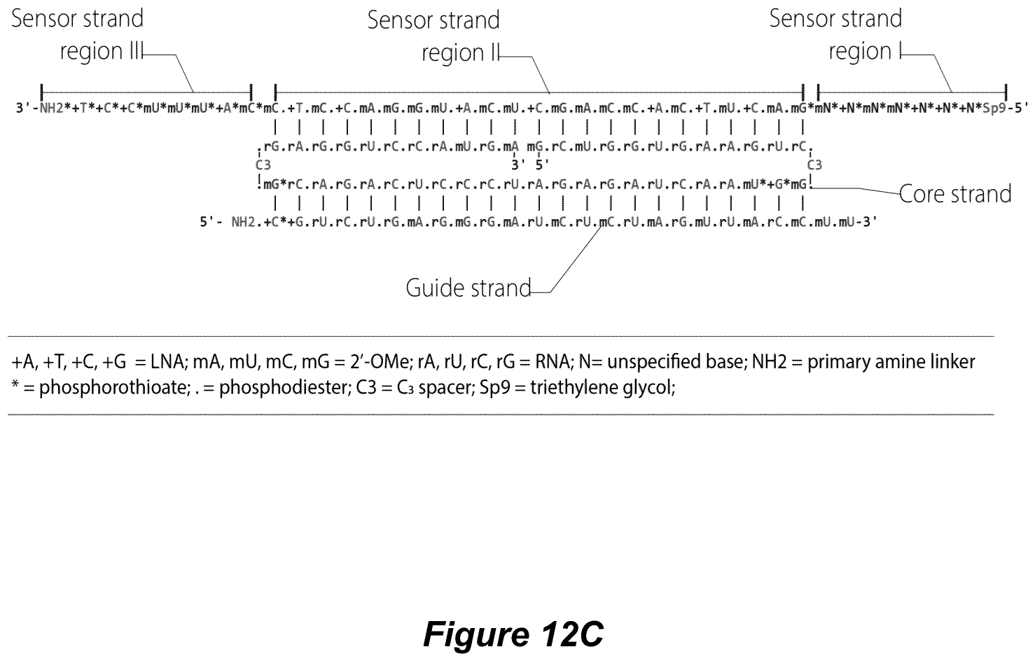

As illustrated in FIG. 12, in some embodiments, the core strand has one or more of the following features.

The 5′ and 3′ termini of the core strand have one or more of the following features:
a. The terminal base on the 5′ is a 2′-F, 2′-O-methyl, or other modified base that resists nuclease cleavage.
b. The terminal base on the 3′ is a 2′-F, 2′-O-methyl, or other modified base that resists nuclease cleavage.
c. The three terminal bases on the 5′ have the pattern MRM, where M is a modified base (2′-O-methyl, 2′-F), and R is an RNA base.
d. The three terminal bases on the 3′ have the pattern MRM, where M is a modified base (2′-O-methyl, 2′-F), and R is an RNA base.
e. The three terminal bases on the 3′ and 5′ do not have consecutive PS backbone modifications.
f. The portions of the core strand that are base-paired with the sensor strand have an alternating chemical modification pattern (MR)n.
g. In the above where M is a chemically modified base that does not decrease Tm of the duplex when compared to the equivalent RNA base.
h. Any combination of above where both the 5′ and the 3′ ends of the core strand have at least one of the features from a-g.
i. The 3′ and 5′ regions I and III of the core strand that are base-paired with the sensor strand are:
   (a) Entirely made out of the pattern (M)n, where M is a 2′-O-methyl or 2′-F modified base, or
   (b) at least 50% of the bases in this area are 2′-O-methyl or 2′ F and, up to 30%, 50%, 80% or 100% of the backbone connections are not phosphorothioate.
      The exonuclease blocking region I attached to the 5′ terminus of the core strand (where the three bases base-paired with 5′ terminus of the guide strand) has one or more of the following features:
a. an M*+*M pattern, where M is a 2′ modified base (e.g. 2′-O-methyl or 2′-F), * is a PS backbone connection, and + is an LNA base or other 2′-4′ bridged base,
b. an M*+*+ pattern, as defined above,
c. a +*+*+ pattern,
d. an R*+*M pattern, where R is an RNA base,
e. an R*+*+ pattern,
f. a +*M*M pattern, and
g. the patterns in a-f, where * can be either a PS backbone connection or an unmodified (phosphodiester) backbone connection.
   The exonuclease blocking region II of the core strand has one or more of the following features:
a. an M*R pattern, where M is a 2′ modified base (e.g. 2′-O-methyl or 2′-F), * is a PS backbone connection, and R is an LNA base or other 2′-4′ bridged base,
b. an M*M pattern, as defined above,
c. a +*M pattern,
d. a +*R pattern, where R is an RNA base,
e. a +*+ pattern,
f. an M*+ pattern, and
g. the patterns in a-f, where * can be either a PS backbone connection or an unmodified (phosphodiester) backbone connection.

As used herein, “LNA” means locked nucleic acid, which is widely used in the field. See, for example, glenresearch.com/products/dna-rna-nucleosides-analogs-and-supports/backbone-modification/locked-analog-phosphoramidites.html, and en.wikipedia.org/wiki/Locked_nucleic_acid. Other techniques of base modifications, in addition to LNA modification, 2′-O-methyl modification, and 2′-F modification, are known in the art. See, for example, glenresearch.com/browse/nucleoside-analog-phosphoramidites. In some embodiments, glycol nucleic acids can be used. ^{61,62}

### D. Design Features of Sensor Strand

As illustrated in FIG. 12, in some embodiments, the sensor strand has one or more of the following features. The sensor strand has a 5′ overhang, a 3′ overhang, or both.

Bases of the sensor strand have one or more of the following features: (1) at least 25% of the bases are not RNA or DNA, (2) at least 50% of the bases are not RNA or DNA, (3) at least 75% of the bases are not RNA or DNA, (4) 100% of the bases are not RNA or DNA, (5) at least one of the bases is an LNA or LNA analogue, (6) at least one of the 3 terminal bases are LNA or LNA analogue, (7) 10% to 50% of the bases are LNAs or LNA analogues, (8) 25% to 100% of the bases are LNAs or LNA analogues, (9) bases that are not LNAs are one or more of the following: (a) 2′-o-methyl, (b) 2′-fluoro, (c) 2′-MOE, (d) glycol nucleic acids, 61,62 and (e) other variants shown in glenresearch.com/browse/nucleoside-analog-phosphoramidites.

Phosphorothioate backbone connections can be present at various locations such as between the terminal base and the second to the last base, between the last 3 bases from the 5′ or the 3′ terminus, between the last 5 bases from the 5′ or the 3′ terminus, between the last 8 bases from the 5′ or the 3′ terminus, on 50% of the backbone connections, between all of the bases, and/or at the connection between regions I and II and between regions II and III.

Terminal modifications at the 5′ end, the 3′ end, or both include one or more of the following: (1) a tri or hexa ethylene glycol spacer, (2) a C3 spacer, (3) an inverted dT, (4) an amine linker, (5) other linkers or terminal modifications known in the art such as those listed at eu.idtdna.com/pages/products/custom-dna-rna/oligo-modifications, and www.glenresearch.com/browse/labels-and-modifiers, and (6) modifications may be used to attach the 3′ and 5′ end to other chemical moieties such as antibodies, gold or other metallic nanoparticles, polymeric nanoparticles, dendrimer nanoparticles, small molecules, single chain or branched fatty acids, peptides, proteins, aptamers, and other nucleic acid strands and nucleic acid nanostructures.

As illustrated in FIG. 3, region C of the sensor strand has one or more of the following features: (1) 50% or less of the backbone positions are phosphorothioate (PS) connections, (2) 50% or more of the bases are chemically modified to resist nuclease degradation or increase the melting temperature of the duplex (Tm), (3) 100% of the bases are chemically modified to resist nuclease degradation and increase Tm, (4) about 10%-50% of the bases are LNAs or other chemically modified base with 2′-4′ bridging modifications that substantively increase the Tm. See, for example, glenresearch.com/products/dna-rna-nucleosides-analogs-and-supports/backbone-modification/locked-analog-phosphoramidites.html, and en.wikipedia.org/wiki/Locked_nucleic_acid.

In some embodiments, non-LNA modifications include 2′-o-methyl and 2′-F, as well as other modifications disclosed in glenresearch.com/browse/nucleoside-analog-phosphoramidites. In some embodiments, glycol nucleic acids can be used.^{61,62}

In one aspect, the programmable conditionally activated RNAi (such as Cond-siRNAs) disclosed herein has a transfection concentration at lower than 0.1 nM in mammalian cells, compared to greater than 10 nM of other RNAi molecules known in the art. The Cond-siRNAs are active for an extended period of time such as at least 12 hours, at least 24 hours, at least 36 hours, at least 48 hours, at least 60 hours, at least 72 hours, at least 84 hours, or at least 96 hours. In some embodiments, the Cond-siRNAs are active for up to 30 days, up to 60 days, or up to 90 days.

As used herein, the term “programmable” means that the Cond-RNAi constructs disclosed herein are designed to allow the change of the sequences of input strand without substantial change of the secondary and tertiary structure of the constructs. Additional design principles are disclosed in U.S. Pat. No. 9,115,355, the contents of which are incorporated herein by reference.

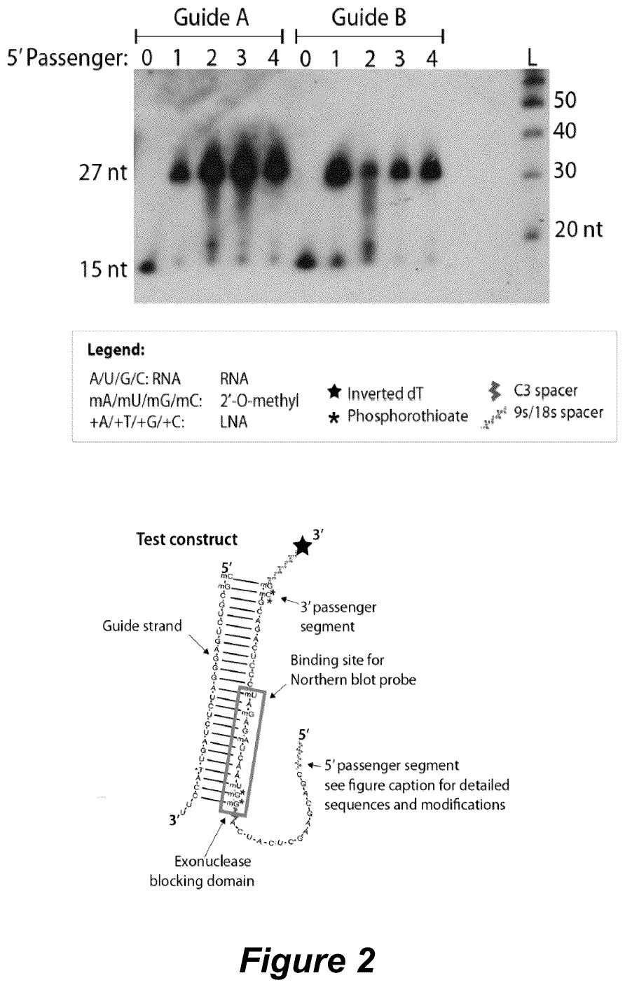

In some embodiments, dissociation of the RNAi duplex from the input-sensor duplex is increased by mismatches or wobble pairs between the core strand and the sensor strand. The RNAi trigger needs to completely dissociate from the waste duplex formed by the input RNA and the sensor strand to have potent RNAi activity. Prior schemes for conditional RNAi often featured activated RNAi triggers that remain attached to the input signal (e.g. an mRNA) via Watson-Crick base pairing^{14,15,17,18,37}. During development of a conditional RNAi trigger, it was found that the connection of Dicer substrates to adjacent chemically protected duplex RNA domains significantly reduced RNAi activity (FIG. 2). A possible cause for this could be binding of a Dicer inhibiting protein such as PACT38 to the extended duplex. In any case, dissociation of the RNAi duplex from the input-sensor duplex was critical in allowing simultaneous optimization of OFF state RNAi suppression and ON state RNAi potency.

In some embodiments, the Cond-siRNA disclosed herein has a single-construct design comprising a sensor duplex attached to a RNAi duplex. The existing designs for conditional RNAi triggers and other DNA circuits have featured either single-construct^{14,15,39} or multi-construct¹⁷ schemes for translating an input sequence into an independent output sequence. Single-construct translators should theoretically be intrinsically more efficient in signal detecting and transduction. However, the disadvantage is that the RNAi trigger must be concealed within the construct, creating an opportunity for spurious RNAi activation due to construct degradation.

In some embodiments, the Cond-siRNAs disclosed herein are chemically modified. For strand displacement sensors, the duplex domain of the sensor strand has LNA modification, 2′-O-methyl modification, or both. In some embodiments, the duplex domain of the sensor strand does not have phosphorothioate (PS) modifications. In some embodiments, either or both termini of the core strand or the protecting strand are modified with either PS or 2′-O-methyl. In some embodiments, thermodynamically stabilizing modifications generally improve suppression of background activation, while thermodynamically destabilizing modifications such as PS backbones can increase spurious activity when used extensively in duplex regions. In some embodiments, the chemical modifications are in the toehold domain, including LNA modification, 2′-O-methyl modification, PS modification, or a combination thereof. These modifications in the toehold domain improve the base-pairing affinity and nuclease resistance of the single stranded overhang.

As used herein, the binding partner to the sensor strand can also be called the “protecting strand”. The protecting strand is the strand that is homologous to the input strand. The protecting strand can be the core strand. The sensor strand is the strand that is complementary to the input strand. Input binding to the sensor strand displaces the protecting strand.

In some embodiments, the chemical modifications include: (i) a sensor strand is modified with LNA and 2′-O-methyl, where the single stranded toehold region has PS backbone modifications but the base-paired duplex region does not have PS backbone modifications, (ii) either or both termini of a protecting strand have 2′-O-methyl modifications, or both (i) and (ii).

In some embodiments, the 3′ and 5′ terminal regions of the core strand are PS modified or 2′-OMe modified such that the Cond-siRNA construct is highly stable when base-paired with the sensor strand but vulnerable to degradation when unpaired. The degradation of single stranded overhangs can be stopped at the ends of duplexes by exonuclease blocking domains.

Complementing targeted drug delivery with self-regulating “smart drugs” that can activate or deactivate RNAi activity in response to cell-specific biochemical signals provide the means to overcome current limitations. For example, an RNAi smart drug that is able to detect and respond to viral RNA transcripts could potentially ablate persistent viral infections by killing virus-infected cells via viral-RNA-activated silencing of survival-essential host genes. This pharmacodynamics centered approach is well suited to the application of nucleic acid switches based on toehold mediated strand displacement, as these switches can sense and respond to specific base sequences in DNA or RNA inputs³.

In one aspect, the Cond-siRNA disclosed herein is a self-assembled molecular mechanical transducer composed of a sensor strand, a guide strand, and a core strand, as shown in FIGS. 1a, 1c, and 1d. These three strands base-pair together to form a double crossover construct²⁰ consisting of a 23-base pair (bp) sensor duplex coupled to a 23-bp RNAi duplex. In the assembled configuration, the sensor inhibits enzymatic processing of the RNAi duplex, thereby keeping RNAi activity switched OFF in the mammalian cytosol (FIG. 1a).

In some embodiments, the Cond-siRNA constructs are further modified by chemical modifications such that their switching performance is improved. Specifically, the Cond-siRNAs disclosed herein have a reduced unwanted RNAi activity when the constructs are in their OFF state and improved switching from OFF to ON state in the presence of cellular RNA transcripts with the correct sequence.

The inhibition of enzymatic processing of the RNAi duplex occurs in multiple ways, which are synergistic in combination. For example, the position of the sensor duplex has large overlaps with the space required by Dicer's dsRBD and endonuclease domains during binding to the RNAi duplex (FIG. 1a, right)²¹. In another example, Dicer's PAZ domain has stabilizing interactions with the 5′ terminal phosphate and 3′ two-base overhang on the ends of canonical dsRNA triggers²². The assembled Cond-siRNA prevents these interactions because the core strand's 5′ and 3′ termini extend past the ends of the RNAi duplex into the middle of the sensor duplex (FIGS. 1c, 1d). The RNAi duplex is 23-bp long. This makes it too long to bypass Dicer cleavage for RNAi loading via alternative pathways²³. In addition, interactions with RNAi pathway proteins required by alternative loading pathways (TRBP and Argonaute(1-4)²¹) are also likely hindered by the construct's unusual tertiary structure. Additionally, to prevent premature degradation of the sensor duplex or unintended separation of the sensor from the RNAi duplex, the entire sensor strand and other key sites on the construct are stabilized thermodynamically and protected from nuclease activity by extensive chemical modifications²⁴ (FIGS. 1c, 1d).

To release and activate the RNAi duplex, a cellular RNA transcript induces separation of the sensor strand from the core strand via toehold mediated strand displacement (FIG. 1b). In some embodiments, the cellular RNA transcript is from an internal source of the subject, such as a metabolism product. Displacement can start from a toehold formed at the 3′ or 5′ terminus of the sensor strand. The sequence specificity of input detection is enforced in the toehold region by the strong dependence of strand displacement kinetics on toehold stability²⁵, and in the duplex region by the need to displace the fully complementary core strand.

After separation from the sensor strand, the displaced regions of the core strand become 3′ and 5′ overhangs extending from the RNAi duplex. The overhangs are degraded by cytosolic nucleases²⁶ (FIG. 1b V and FIG. 2), which are stopped at the ends of the RNAi duplex by chemically modified nuclease blocking domains (FIG. 3a, yellow highlights in construct I.1). This leaves an active RNAi trigger for Dicer processing and RISC loading (FIG. 1b, VI, siRNA).

Strand compositions used in the intracellular degradation of chemically modified single stranded overhangs were as follows:

Sequences (5′->3′)
Guide A:
(SEQ ID NO: 76)
mCmG C GUCUGAGGGAUCUCUAGU UACCUU
Guide B:
(SEQ ID NO: 77)
mCmG+CGUCUGAGGGAUCUCUAGU+TACCUU
3′ passenger segment:
(SEQ ID NO: 78)
cccucagacg mc*mg*9s idT
5′ Passenger segments
0 (control):
(SEQ ID NO: 79)
c3 mG*mG*mU AACUmAGAmGAm U
1:
(SEQ ID NO: 80)
C G A C G A G C U C A U C A
c3mG*mG*mU AACUmAGAmGAm U
2:
(SEQ ID NO: 81)
18s *C*G*A*C*G*A*A*G*C*U*C*A*U*C*
c3mG*mG*mU AACUmAGAmGAm U
3:
(SEQ ID NO: 82)
18s *C*G*A*C*G*A*A*G*C*U*C*A U C
c3mG*mG*mU AACUmAGAmGAm U
4:
(SEQ ID NO: 83)
18s *C*G*A*C G A G C U C A U C
c3mG*mG*mU AACUmAGAmGAm U
Northern probe:
(SEQ ID NO: 84)
ATCTCTAGTTACC

L: Ambion decade marker

### Abbreviations

9s: triethylene glycol spacer
18s: hexaethylene glycol spacer
C3: C3 spacer
idT: inverted dT
*: phosphorothioate backbone connection.

As shown in FIG. 2, samples with guide strand A had sufficient loading and exposure to visualize all bands. Lane 0 shows position of control strand with no overhang (15 nucleotides). Passenger 1 has a reduced amount of full length passenger strand with a single detectable band at ˜15 nt, indicating rapid processive degradation of the overhang. Passengers 2 and 3 had multiple bands and streaks throughout the size range between 15 and 27 nt, indicating a slow, non-processive loss of nucleotides, consistent with presence of PS backbone connections throughout the overhang. Passenger 4 showed higher amounts of full length product versus 0, with two bands visible near 15 nt, indicating a slower initial degradation rate due to end protection, before rapid processive loss of the overhang once 5′ terminal protection is lost.

Several Cond-siRNAs were designed with chemical modification motifs similar to those used to protect antisense oligonucleotides (ASO) and other oligonucleotide therapeutics²⁴. Construct I.1 (FIG. 3a) was programmed to detect a conserved HIV (tat/rev) mRNA gene sequence²⁷. The activator sequences for constructs I and II are shown in Table 1 below. Regions meant to align with sensor strand are bold and italicized; regions meant to align with toehold are underlined; segments complementary to the sensor strand are in upper case; and segments mismatched with sensor strand are in lower case.

TABLE 1
Activator Sequences for Constructs I and II

| Fully | GcuAUGGCAGGAAGAAGCGGAGACAGCGACGAAGAGcuca |
|---|---|
| matched | ucagaacagucggcgcaagccuuuuuu (SEQ ID NO: |
| | 85) |
| Full | Gcgaacggcauuagcggcacaagagacgacggaagaguca |
| mismatch | ucagaacagucggcgcaagccuuuuuu (SEQ ID NO: |
| | 86) |
| 5′ | GcuAUGGCAGGAAGAAGCGGAGACAGCGcaauccuuauca |
| mismatch | ucagaacagucggcgcaagccuuuuuu (SEQ ID NO: |
| | 87) |
| 3′ | GcucacugAGGAAGAAGCGGAGACAGCGACGAAGAGcuca |
| mismatch | ucagaacagucggcgcaagccuuuuuu (SEQ ID NO: |
| | 88) |

Construct III.1 (FIG. 3a) was programmed to detect an acute myeloid leukemia (AML) associated fusion oncogene sequence (CBFB-MYH11)^{28,29}. The activator sequences for constructs III and IV are shown in Table 2 below. Regions meant to align with sensor strand are bold and italicized; regions meant to align with toehold are underlined; segments complementary to the sensor strand are in upper case; and segments mismatched with sensor strand are in lower case.

TABLE 2
Activator Sequences for Constructs III and IV

| | |
|---|---|
| Fully | GacaggucucaucgggAGGAAAUGGAGGUCCAUGAGCUG |
| matched | GAGAAGUCCAAgcgggcgcaagccuuuuuu |
| | (SEQ ID NO: 89) |
| Full | gcuauggcaggaagaagcggagacagcgacgaagagcuc |
| mismatch | aucagaacagucggcgcaagccuuuuuu |
| | (SEQ ID NO: 90) |
| MYH11 | ucagcuccaaggaugacgugggcaagaacGUCCAUGAGC |
| parental | UGGAGAAGUCcaagcgggcgcaagccuuuuuu |
| | (SEQ ID NO: 91) |
| CBFβ | gacaggucucaucgggAGGAAAUGGAGGcaagaagacaa |
| parental | caagacccuaguccugggcgcaagccuuuuu |
| | (SEQ ID NO: 92) |

To allow direct readout of RNAi activity, both constructs I.1 and III.1 targeted a Renilla luciferase mRNA bearing a biologically irrelevant target sequence in its 3′ UTR (from the U5 region of HIV).

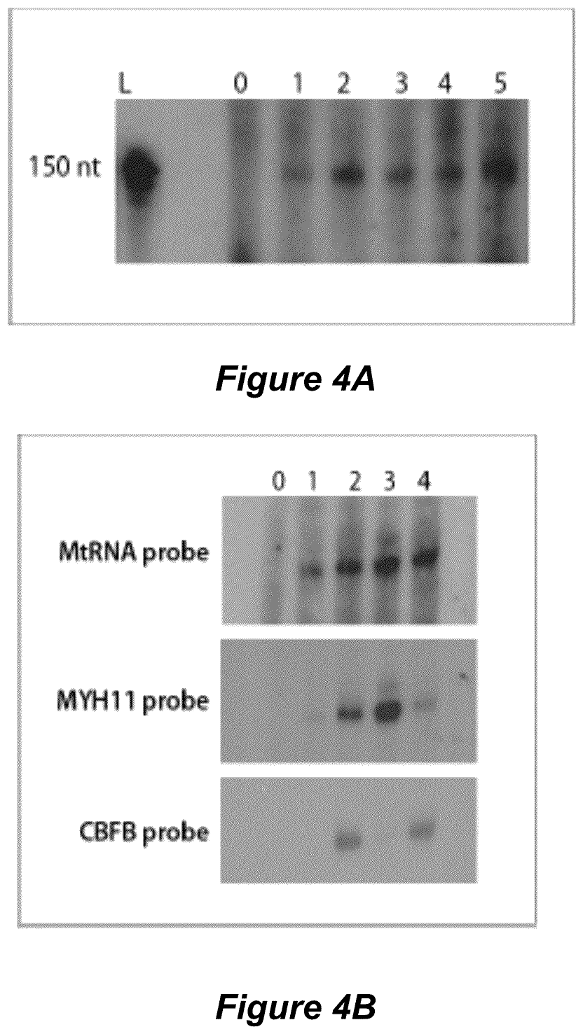

To predict realistic molecular conformations, atomistic molecular dynamics (MD) simulations³⁰ of I.1 and III.1 were conducted in explicit solvent using a hybrid Amber force field that uses a combination of previously published parameters³¹⁻³³ for describing the chemical modifications used in our design. MD optimized models for both constructs (FIGS. 1c, 1d) show that there are structural distortions in the sensor and RNAi duplexes compared with ideal A-form RNA helical parameters (FIG. 4). The distortions are attributable to (1) extensive incorporation of chemical modifications and (2) structural strain intrinsic in the double crossover motif (FIGS. 1c, 1d, 4, and Tables 3a and 3b). Tables 3a and 3b below show average base-pair parameters of sensor and siRNA duplexes over 5 nanoseconds of Molecular Dynamics trajectories. The mean and standard deviation values for each base-pair parameter for each denoted duplex was calculated from the data shown in FIG. 4. For comparison, mean and standard deviations were also calculated for unconnected RNA duplexes with the same sequence composition as the siRNA and sensor duplexes in the HIV and AML constructs (both constructs had the same sequence in the siRNA duplex).

TABLE 3a

| | | | Degrees | Å | Degrees | Degrees | Degrees | Degrees |
|---|---|---|---|---|---|---|---|---|
| | | | Buckle | Rise | Twist | Opening | Propeller | Roll |
| RNA | siRNA duplex | Mean | 0.87 | 2.74 | 31.59 | 0.78 | −12.57 | 7.71 |
| duplexes with | | STD | 12.00 | 0.52 | 4.14 | 4.88 | 8.46 | 6.17 |
| no modified | I.1 sensor duplex | Mean | −3.49 | 2.66 | 32.32 | 0.50 | −12.56 | 7.91 |
| nucleotides | | STD | 10.99 | 0.56 | 4.58 | 4.64 | 8.35 | 6.50 |
| | II.1 sensor duplex | Mean | −4.36 | 2.61 | 31.57 | 0.01 | −12.40 | 8.44 |
| | | STD | 11.12 | 0.89 | 7.28 | 4.59 | 8.83 | 6.42 |
| I.1 construct | siRNA | Mean | 0.00 | 2.68 | 31.55 | 0.38 | −12.31 | 7.98 |
| | | STD | 12.47 | 0.63 | 5.02 | 4.96 | 8.71 | 7.14 |
| | Sensor | Mean | 1.54 | 2.95 | 29.54 | −0.42 | −8.47 | 4.09 |
| | | STD | 11.85 | 0.57 | 4.66 | 4.75 | 8.63 | 6.71 |
| II.1 construct | siRNA | Mean | 0.09 | 2.75 | 31.55 | 0.25 | −12.08 | 7.29 |
| | | STD | 9.62 | 0.69 | 5.36 | 4.07 | 9.16 | 6.12 |
| | Sensor | Mean | 1.49 | 2.73 | 29.87 | −0.11 | −10.36 | 5.89 |
| | | STD | 10.14 | 0.70 | 5.67 | 4.35 | 8.44 | 6.22 |

TABLE 3b

| | | | Degrees | Å | Degrees | Degrees | Degrees | Degrees |
|---|---|---|---|---|---|---|---|---|
| | | | Shear | Shift | Slide | Stagger | Stretch | Tilt |
| RNA | siRNA duplex | Mean | 0.00 | 0.02 | −1.68 | −0.06 | −0.09 | −0.04 |
| duplexes with | | STD | 0.33 | 0.67 | 0.49 | 0.42 | 0.14 | 4.66 |
| no modified | I.1 sensor duplex | Mean | −0.02 | 0.01 | −1.63 | −0.09 | −0.10 | 0.34 |
| nucleotides | | STD | 0.31 | 0.66 | 0.51 | 0.42 | 0.13 | 4.86 |
| | II.1 sensor duplex | Mean | −0.04 | 0.10 | −1.68 | −0.08 | −0.05 | 0.17 |
| | | STD | 0.32 | 0.80 | 0.51 | 0.43 | 0.13 | 4.63 |
| I.1 construct | siRNA | Mean | 0.00 | 0.04 | −1.82 | −0.08 | −0.03 | 0.12 |
| | | STD | 0.32 | 0.71 | 0.57 | 0.43 | 0.14 | 4.80 |
| | Sensor | Mean | 0.00 | −0.25 | −2.18 | 0.04 | −0.06 | −0.82 |
| | | STD | 0.33 | 0.69 | 0.52 | 0.43 | 0.14 | 4.87 |
| II.1 construct | siRNA | Mean | 0.00 | 0.05 | −1.82 | −0.05 | −0.04 | 0.06 |
| | | STD | 0.32 | 0.71 | 0.56 | 0.40 | 0.12 | 4.72 |
| | Sensor | Mean | −0.01 | −0.24 | −2.16 | 0.03 | −0.05 | −0.80 |
| | | STD | 0.33 | 0.71 | 0.53 | 0.41 | 0.13 | 4.69 |

Despite distortions, all intended base pairs were maintained throughout simulation, and the relative dispositions of the sensor and RNAi duplexes in both models were consistent with steric protection from Dicer cleavage.

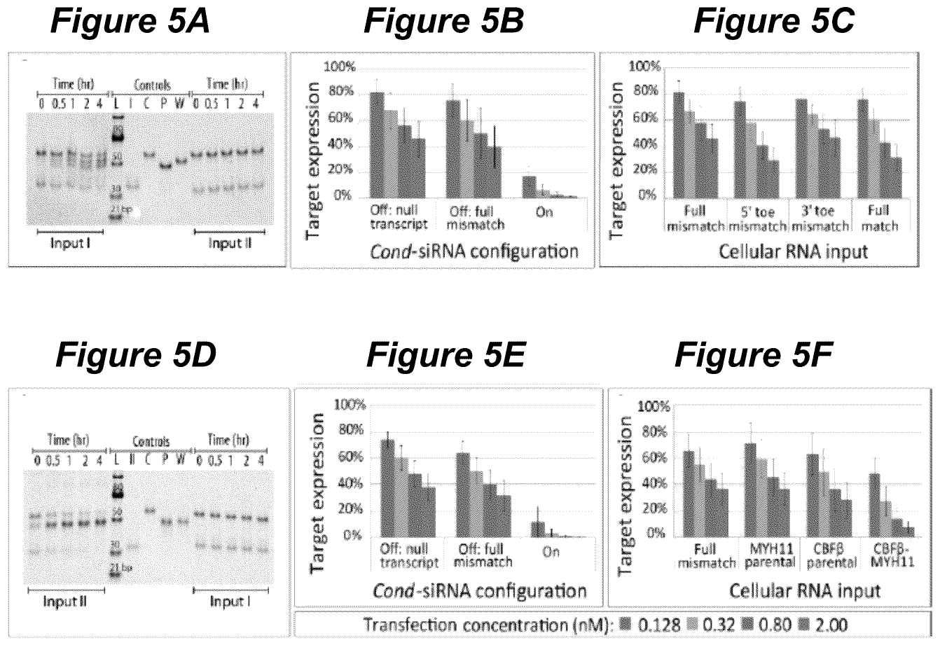

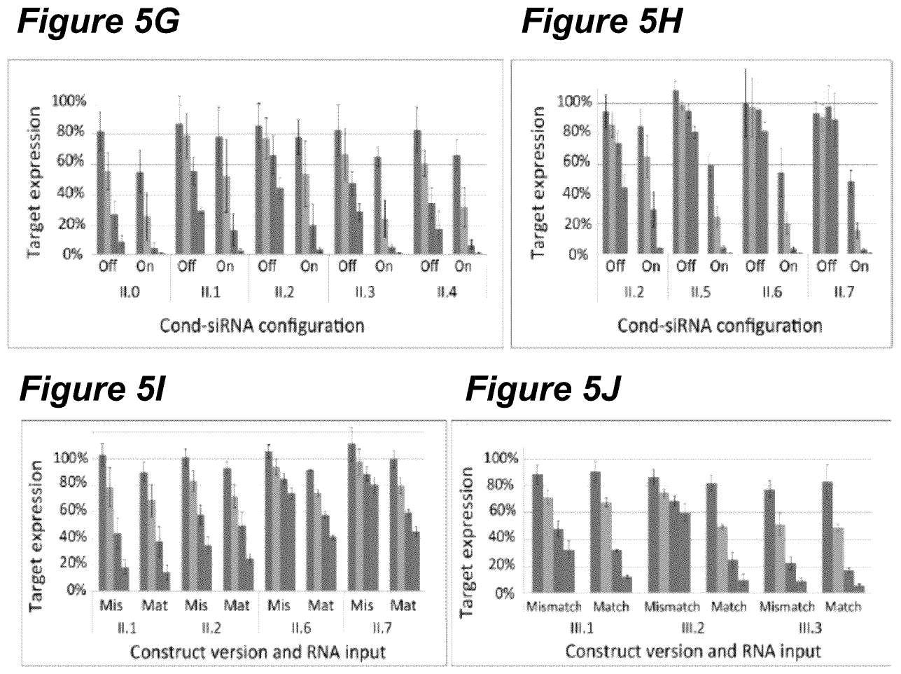

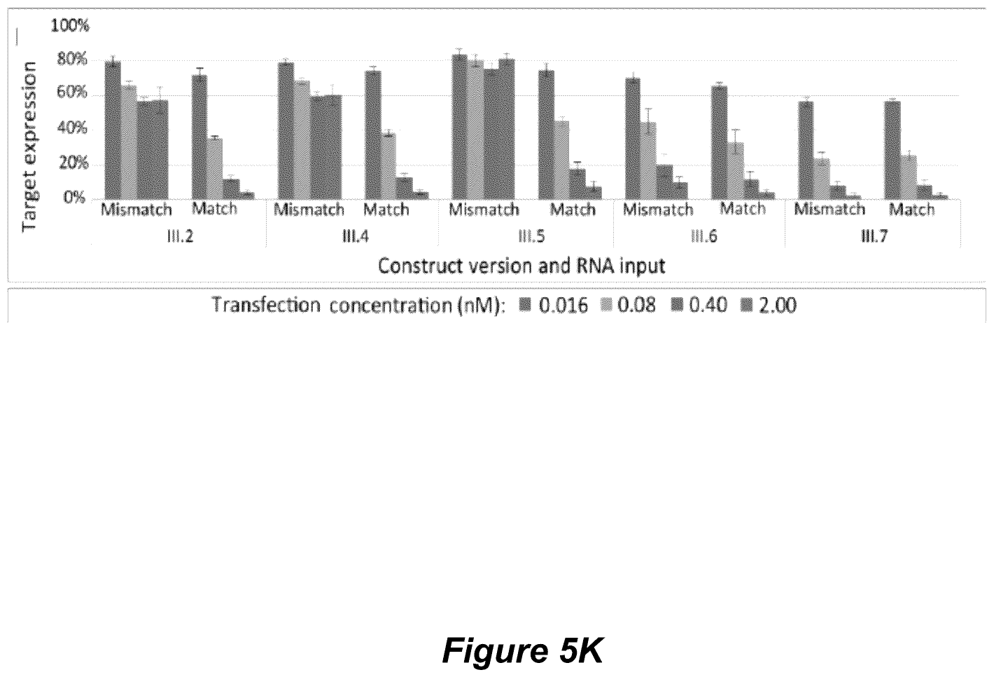

For empirical testing of the constructs, the component strands were purchased from commercial sources and Cond-siRNAs were assembled using thermal annealing in 1×PBS. Non-denaturing polyacrylamide gel (PAGE) analysis showed that both constructs assembled with high purity, were capable of sequence specific detection of input RNA, and could release RNAi duplexes via isothermal strand displacement at 37° C. in 1×PBS buffer (FIGS. 5a, 5d).

The RNAi activities of OFF (guide, core, and sensor strands) and ON (guide and core strands only) state Cond-siRNAs were measured using dual luciferase assays. Varying amounts of constructs were co-transfected into HCT116 cells with fixed amounts of DNA vectors encoding (a) the dual luciferase reporter and (b) short RNA inputs complementary (matched) or noncomplementary (mismatched) sequences to the sensor strand (Tables 1 and 2).

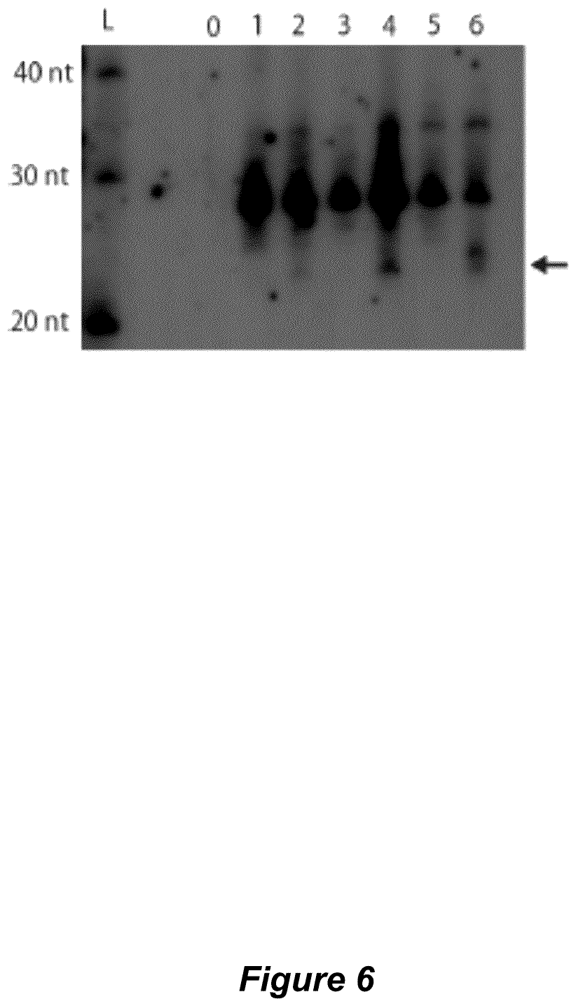

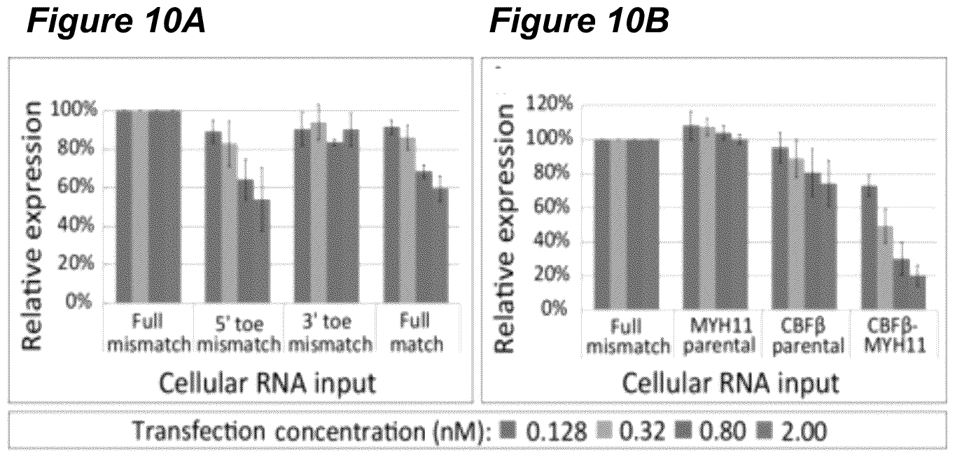

In cells expressing null or fully mismatched inputs, OFF state Cond-siRNAs had significantly reduced RNAi activity while ON state constructs proved to be potent RNAi triggers (FIGS. 5b, 5e corroborated by Northern blot assay results FIG. 6). For RNAi activation, OFF state constructs were transfected into cells expressing matched or mismatched inputs (FIGS. 5c, 5f). Expression levels of input RNA were assessed using Northern blot (FIG. 4). Although fluctuations in the background (OFF state) RNAi activity level reduced the apparent statistical significance when the data was normalized to target expression in cells with no transfect siRNA (FIGS. 5c and 5f), an alternative analysis directly comparing matching and mismatched inputs suggested that there was a significant RNAi activity increase with matching inputs (FIG. 10).

The different sensor designs of I.1 and III. 1 appeared to result in some functional differences in RNAi activation. Construct I.1 had five nucleotide 5′ and 3′ sensor strand overhangs. RNAi activation was unaffected by input mismatches to the 5′ overhang but completely eliminated by mismatches to the 3′ overhang (FIGS. 5c, 5f, and 10). Thus, only the 3′ sensor overhang was functional as a toehold for strand displacement. In the MD simulations, strand displacement from construct I.1′s 5′ toehold would initially proceed towards the interior of the construct while displacement from the 3′ toehold would proceed towards the exterior (FIGS. 1c, 1d). Thus, steric hindrance may play a role in preventing strand displacement from a 5′ toehold. Other factors such as binding site accessibility on the RNA input or differences in base-pairing stability between the 5′ and 3′ toeholds may also contribute to preventing strand displacement from a 5′ toehold.

Construct III.1 showed greater RNAi activation (FIG. 5f) than construct I.1 (FIG. 5c) despite having a similarly potent ON state RNAi trigger (FIGS. 5b, 5e). A plausible explanation is that the greater thermodynamic stability of III.1's eight base 3′ toehold results in faster strand displacement kinetics.

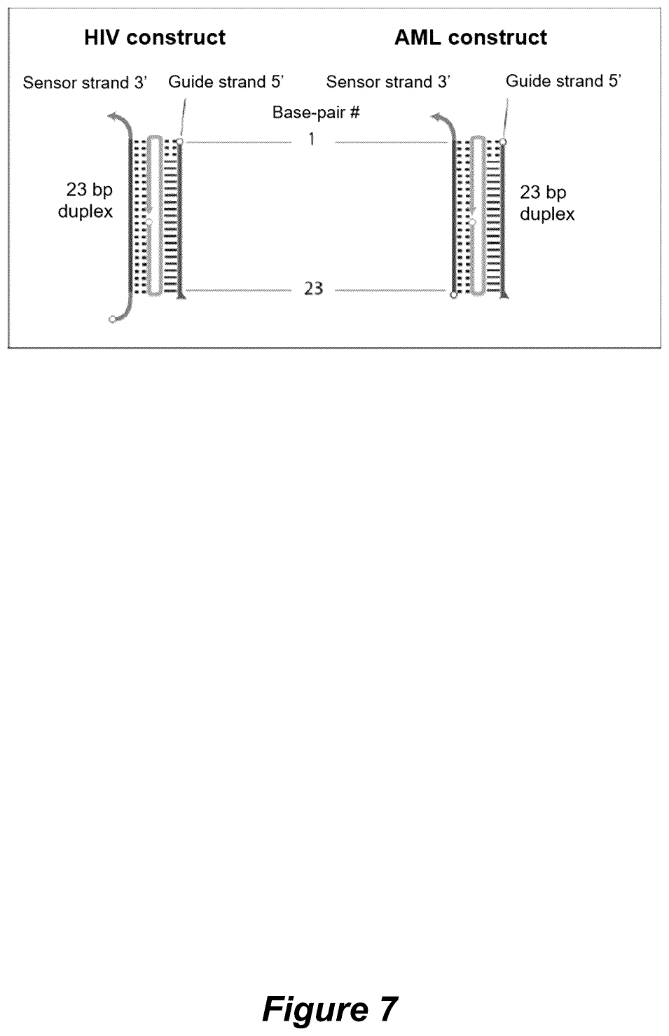

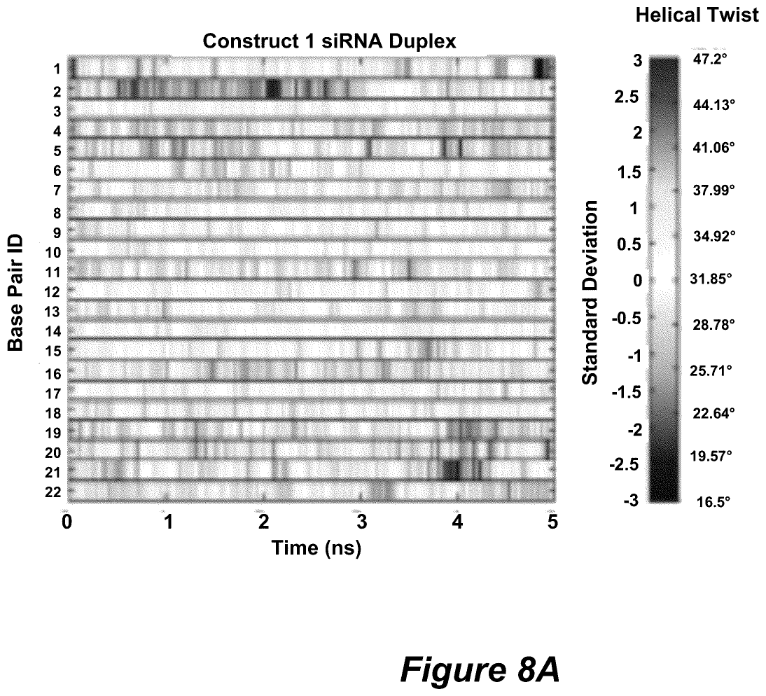

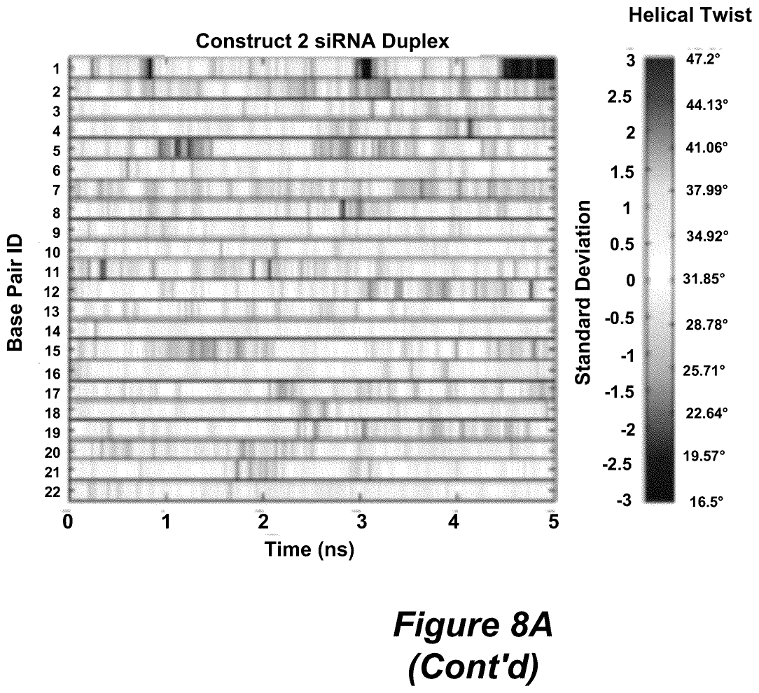

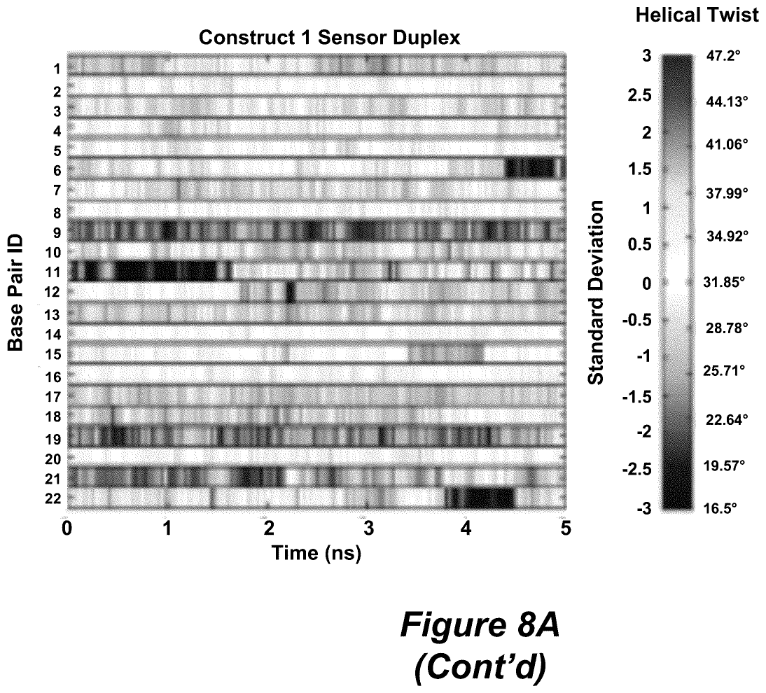

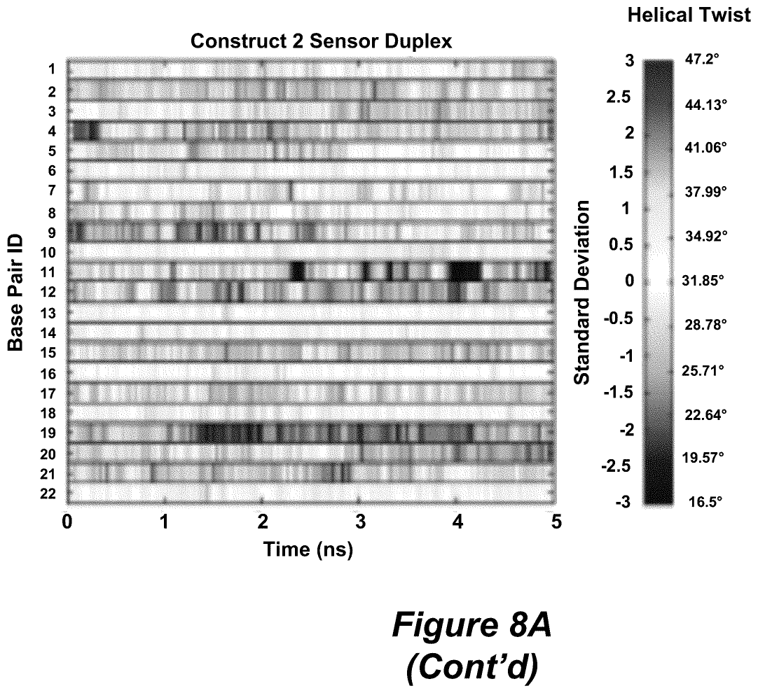

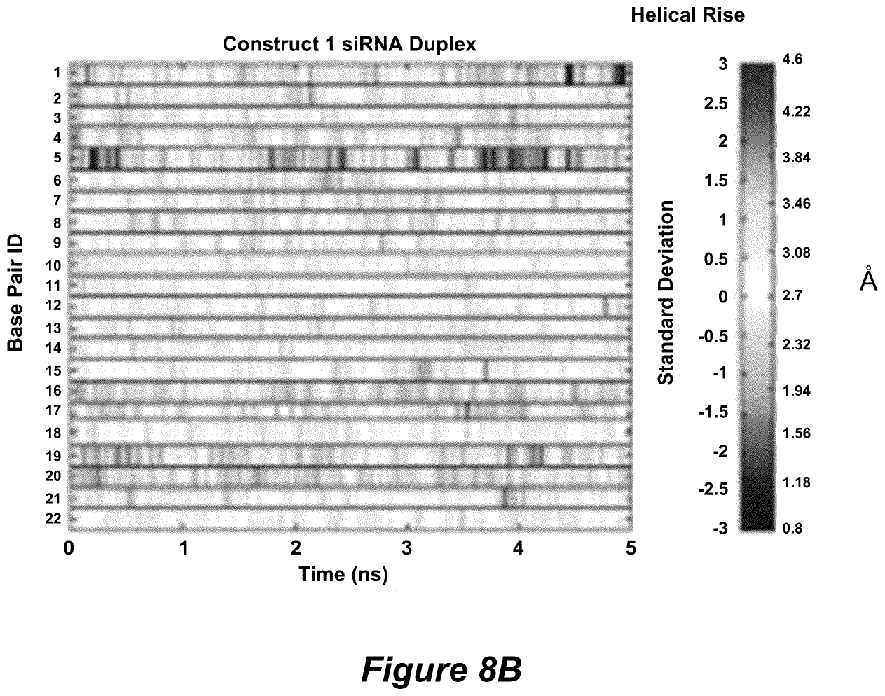

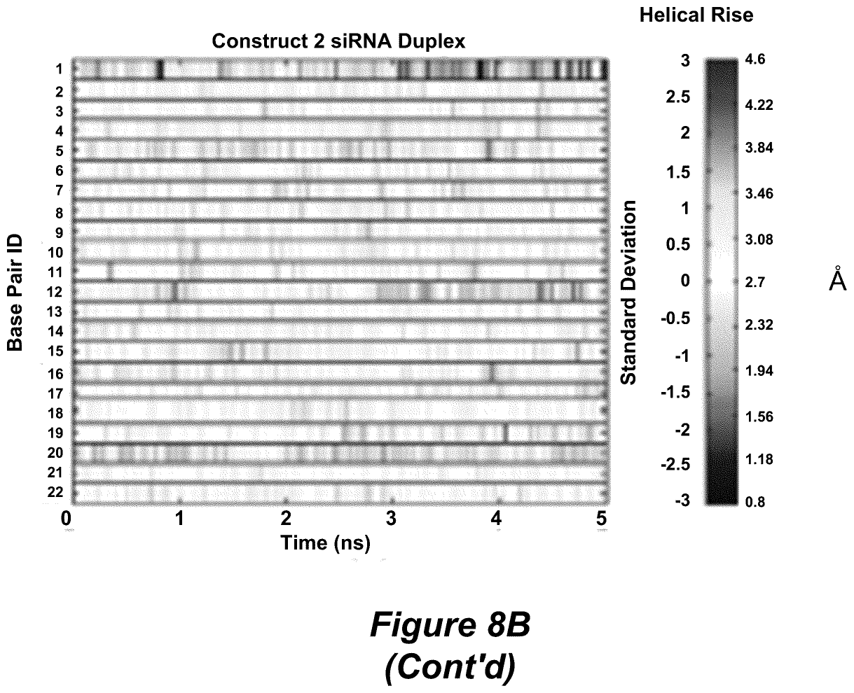

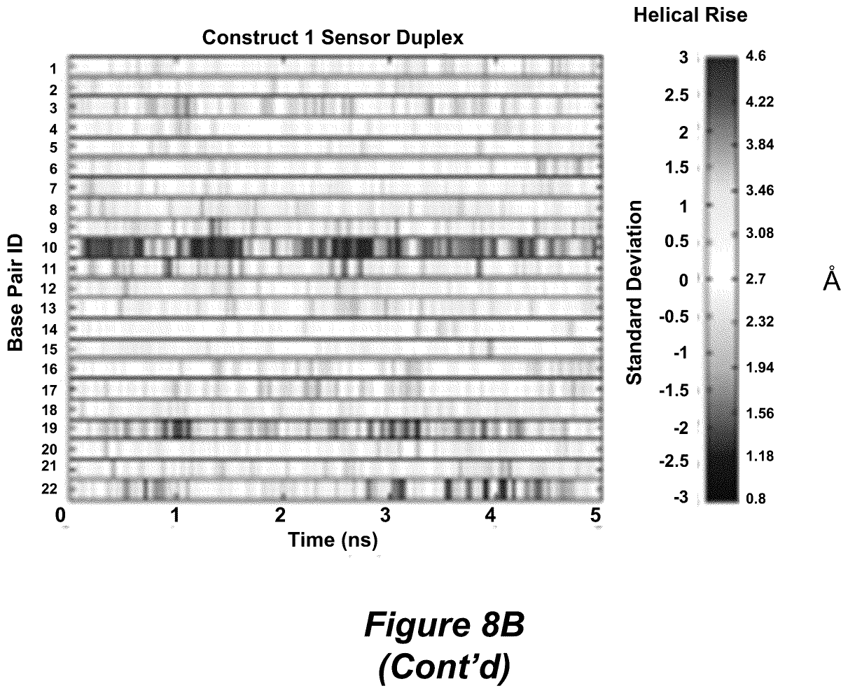

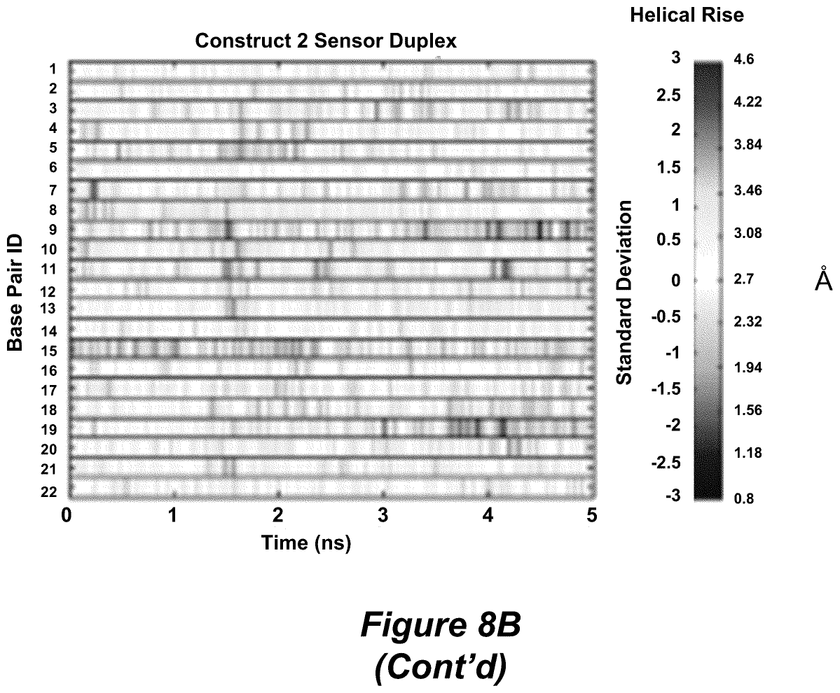

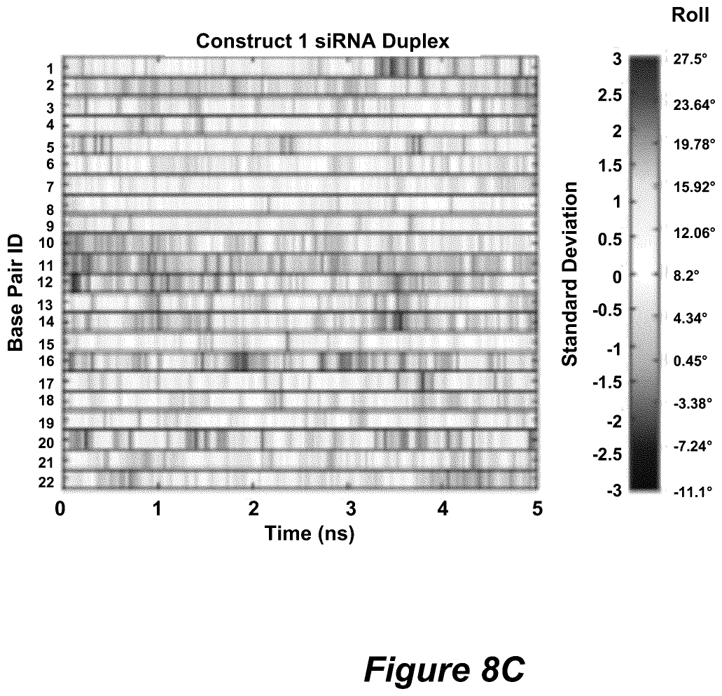

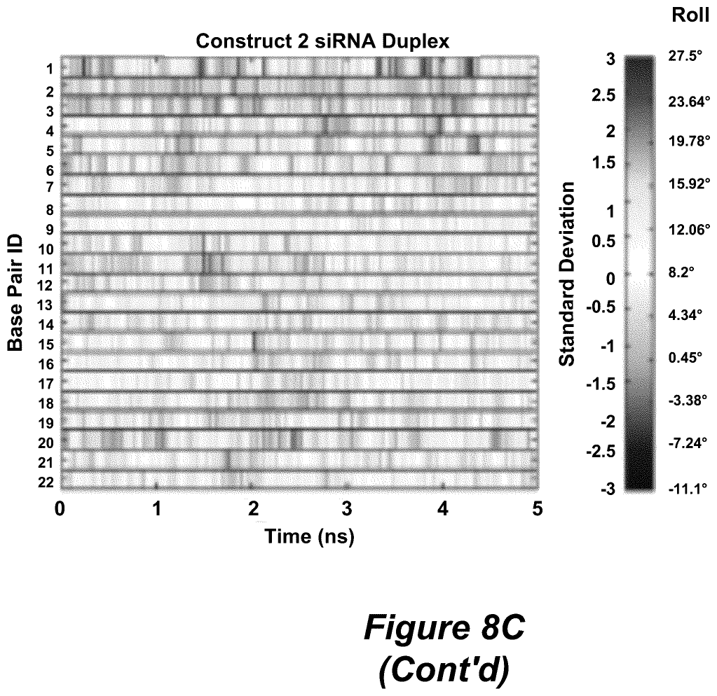

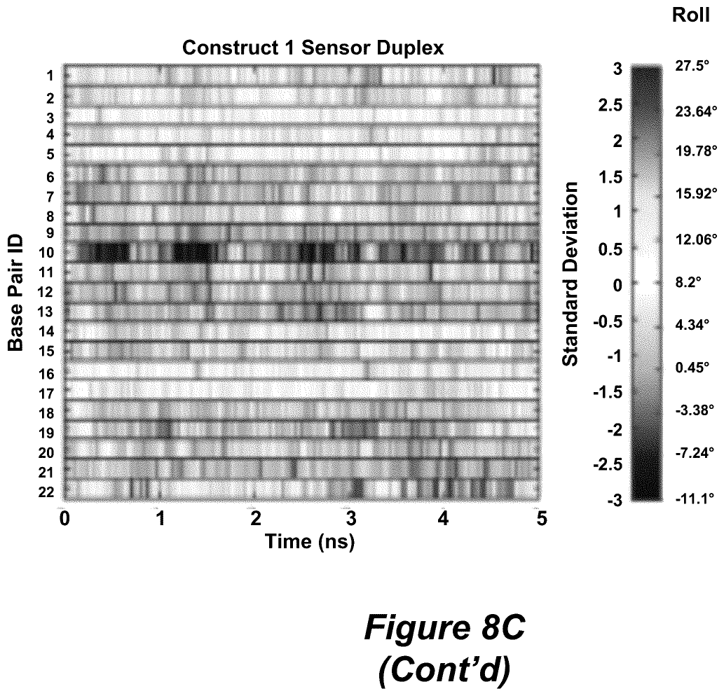

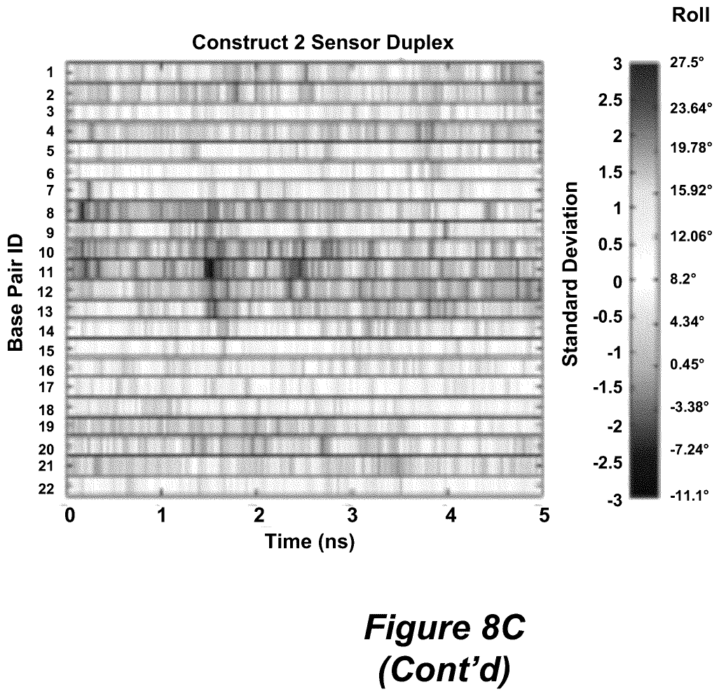

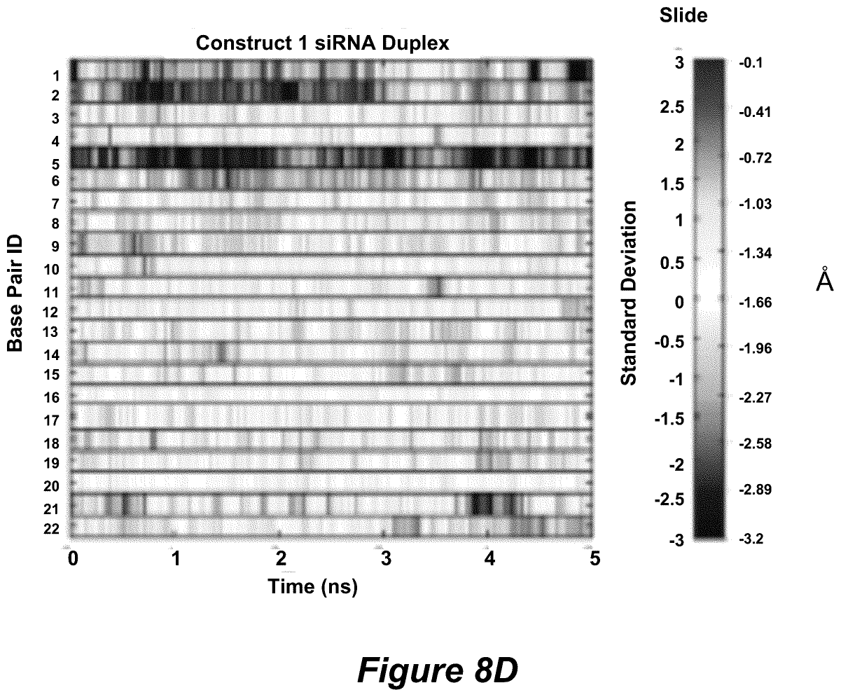

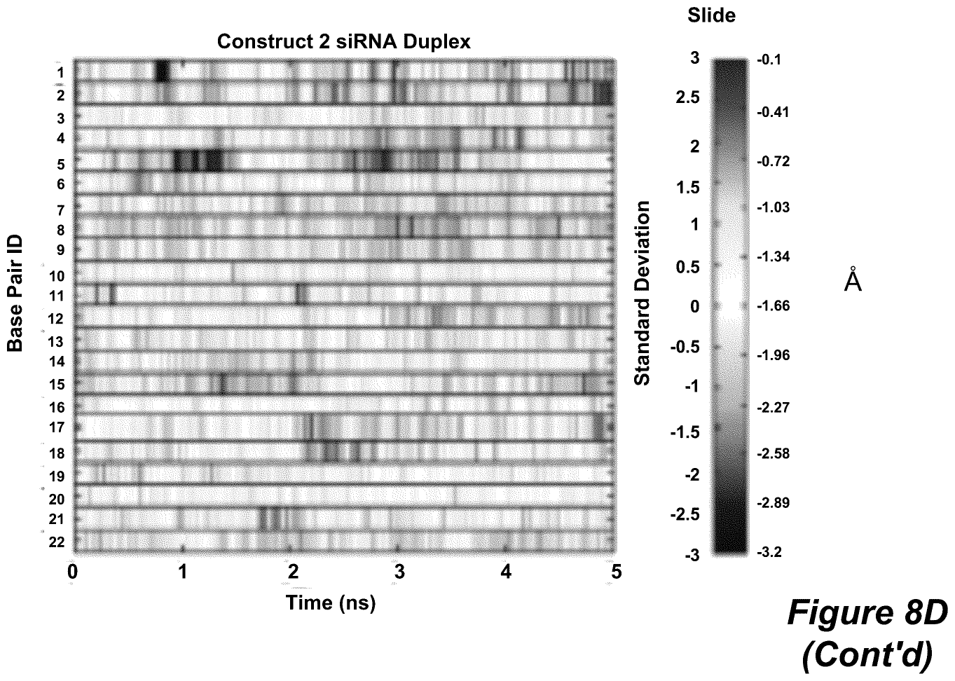

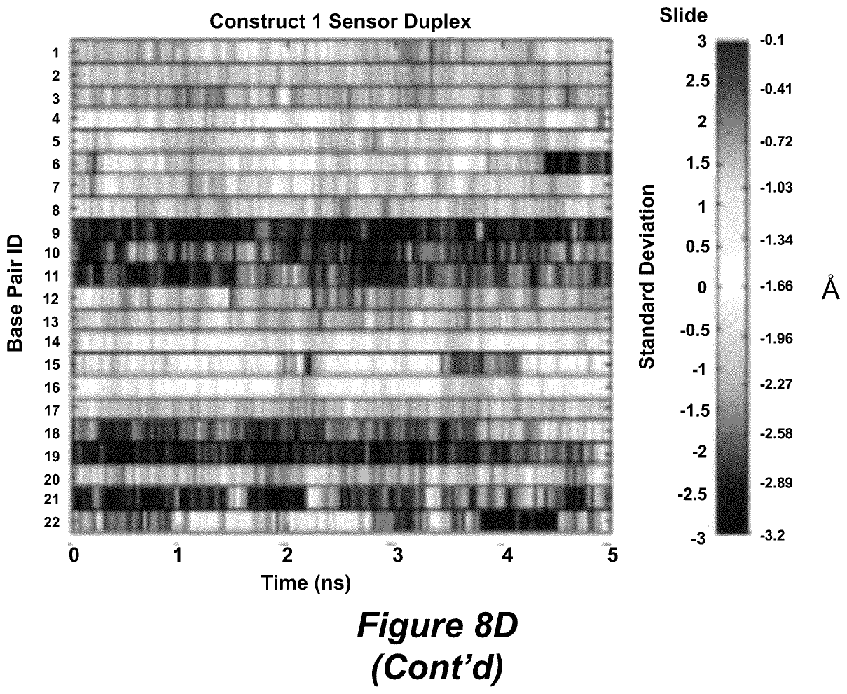

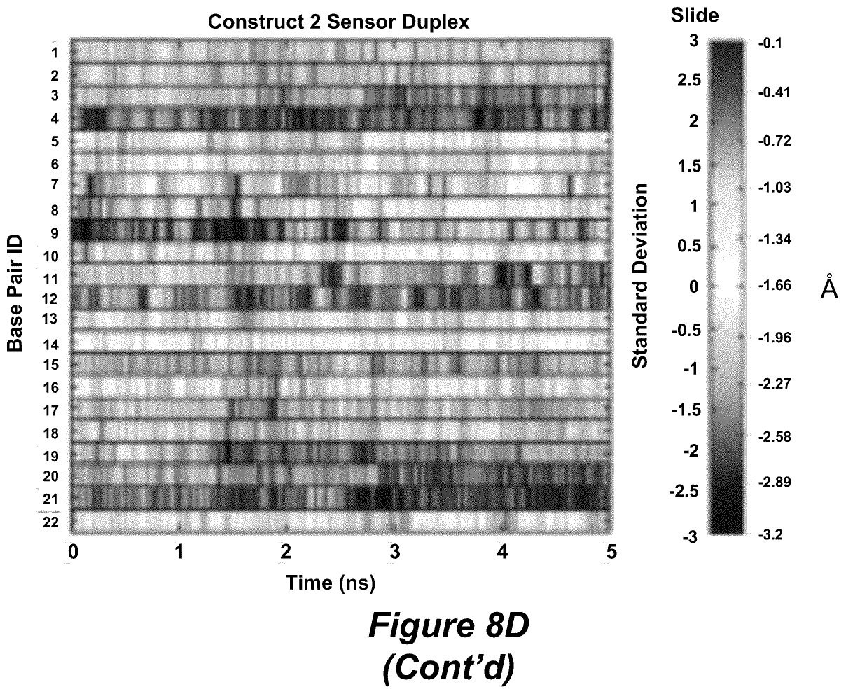

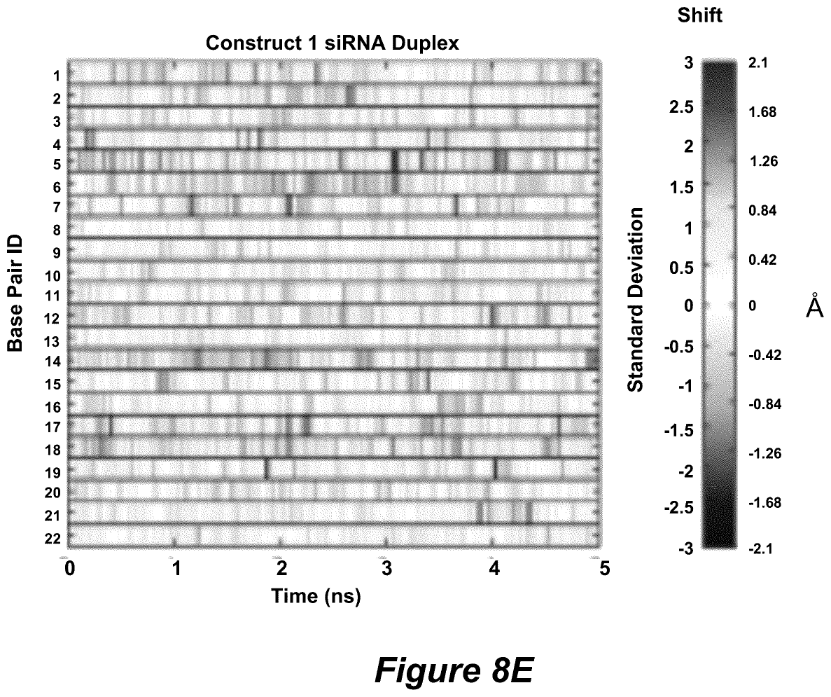

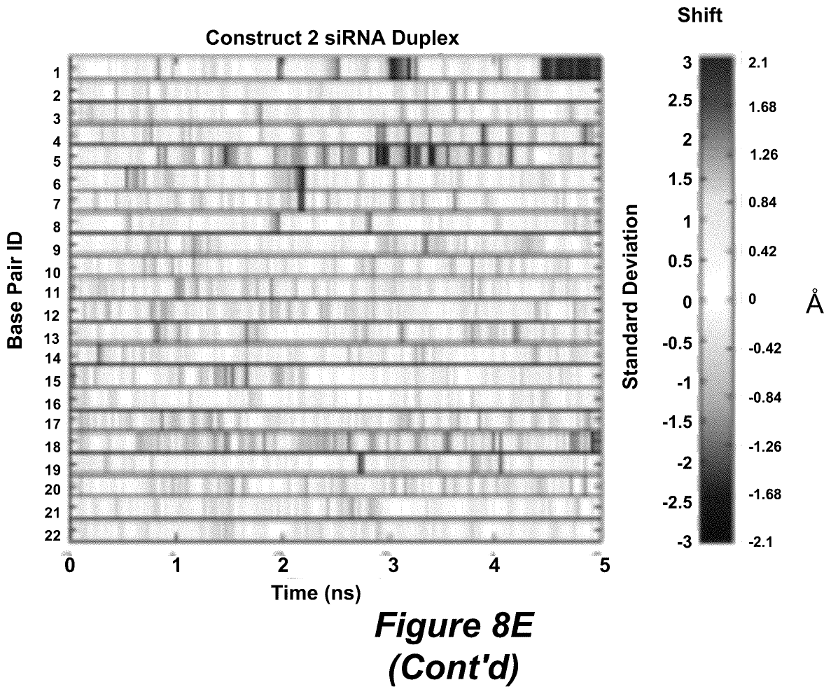

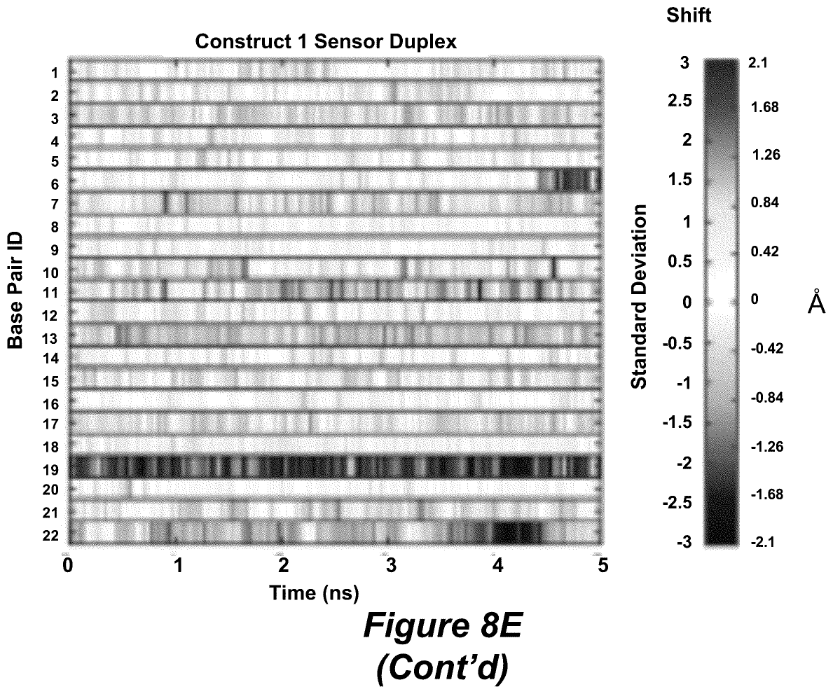

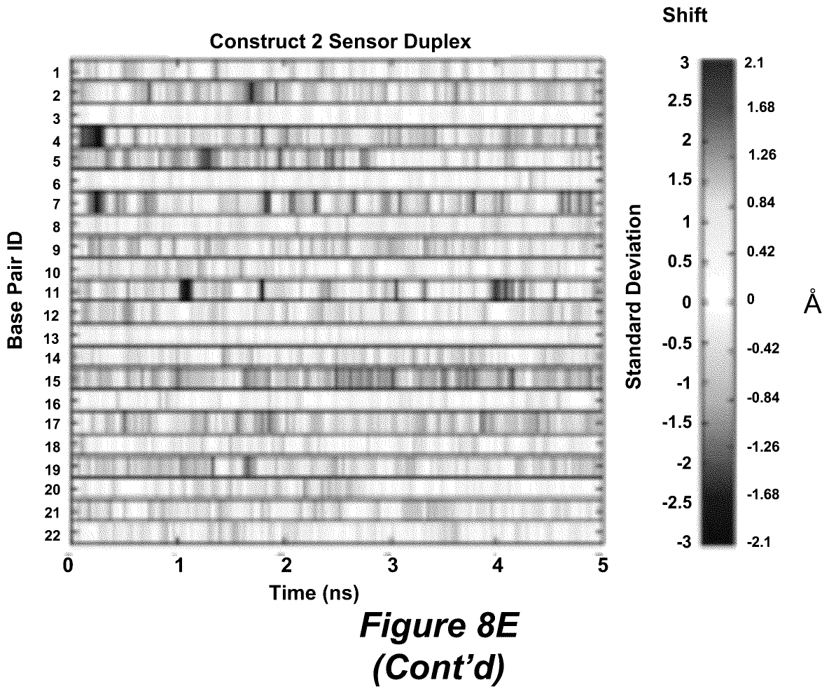

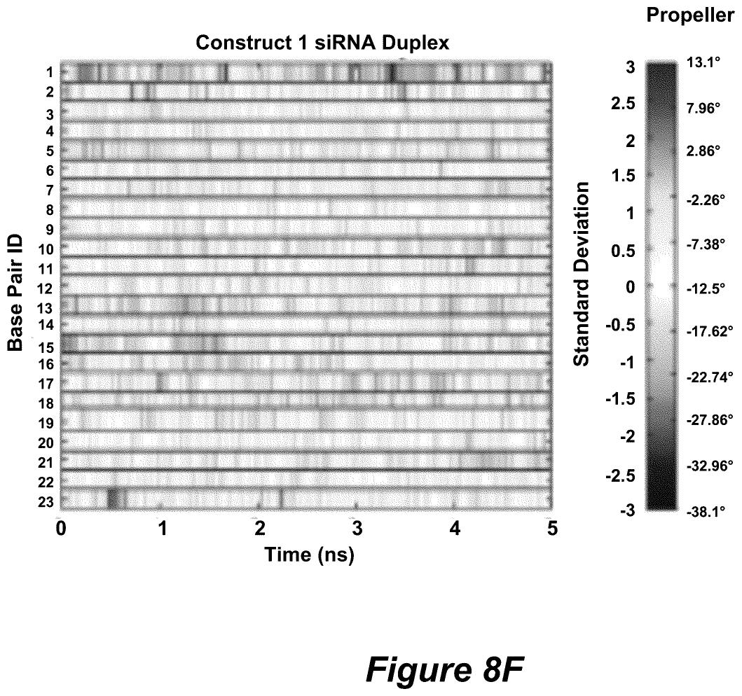

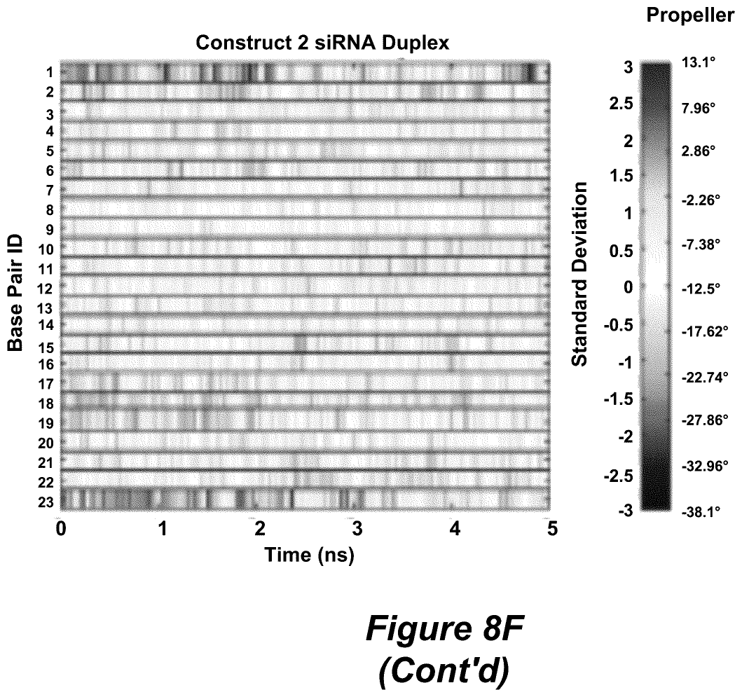

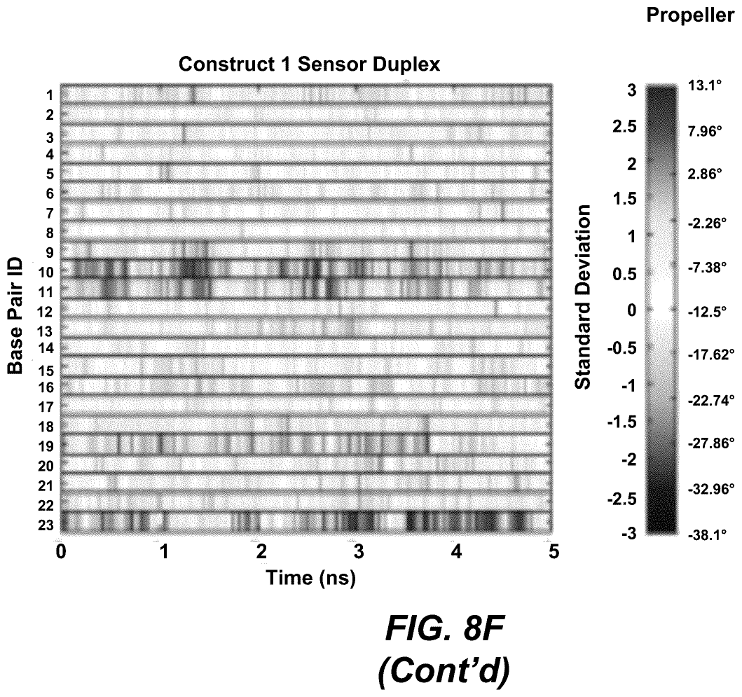

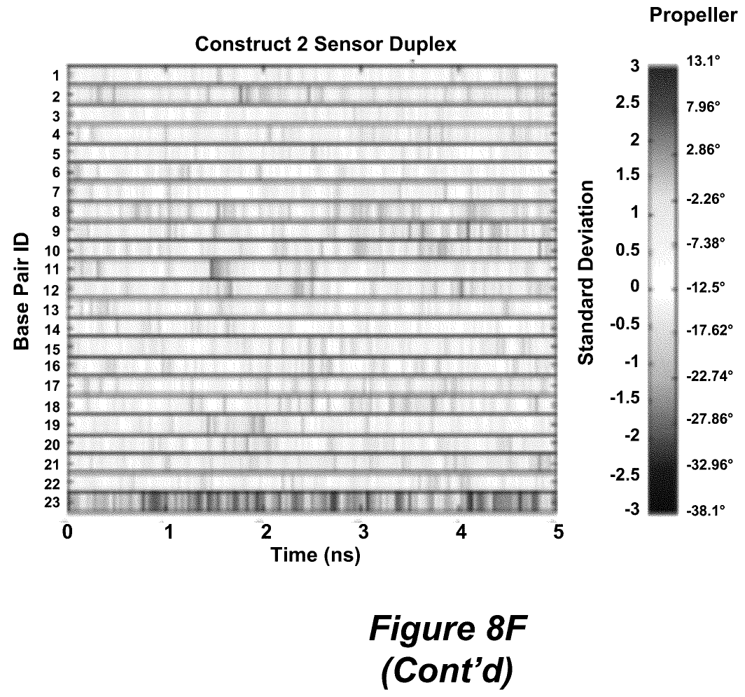

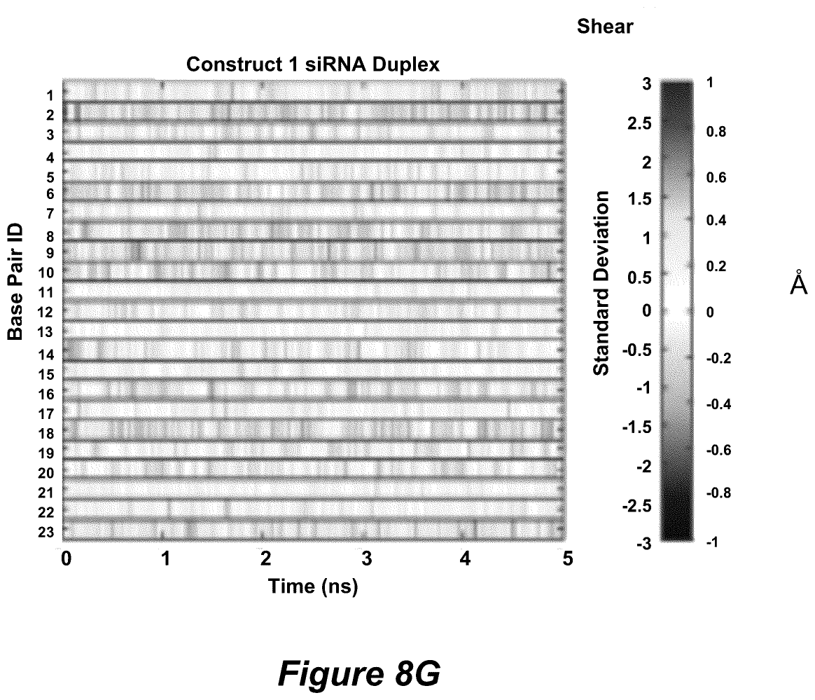

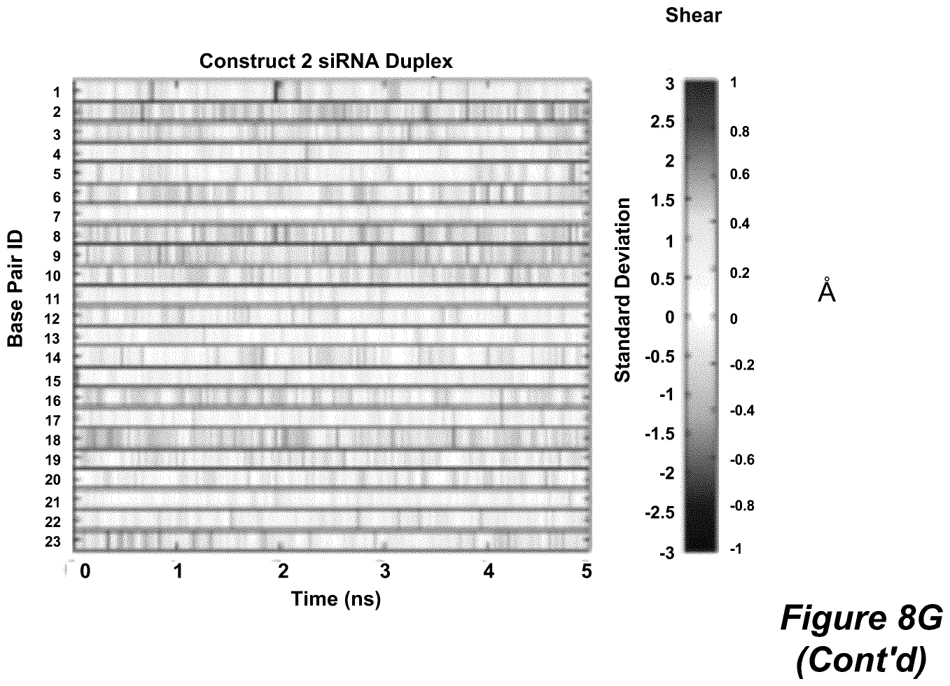

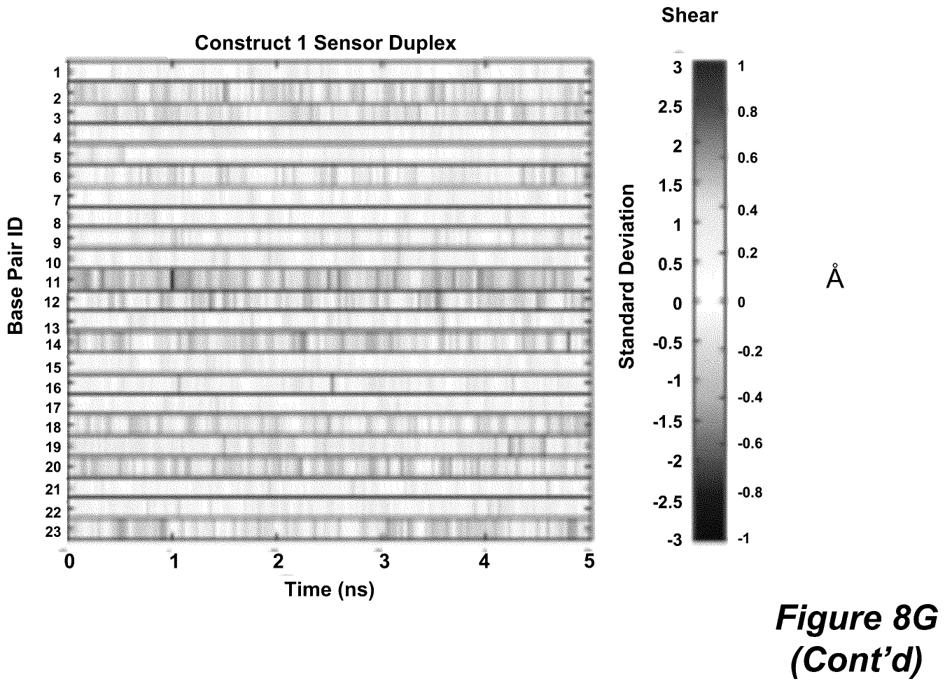

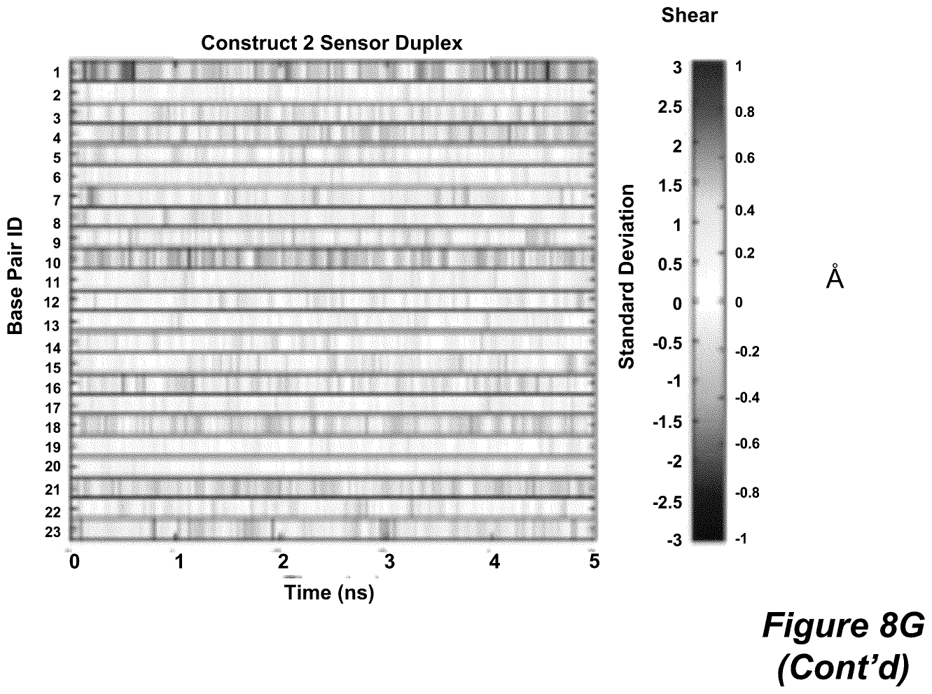

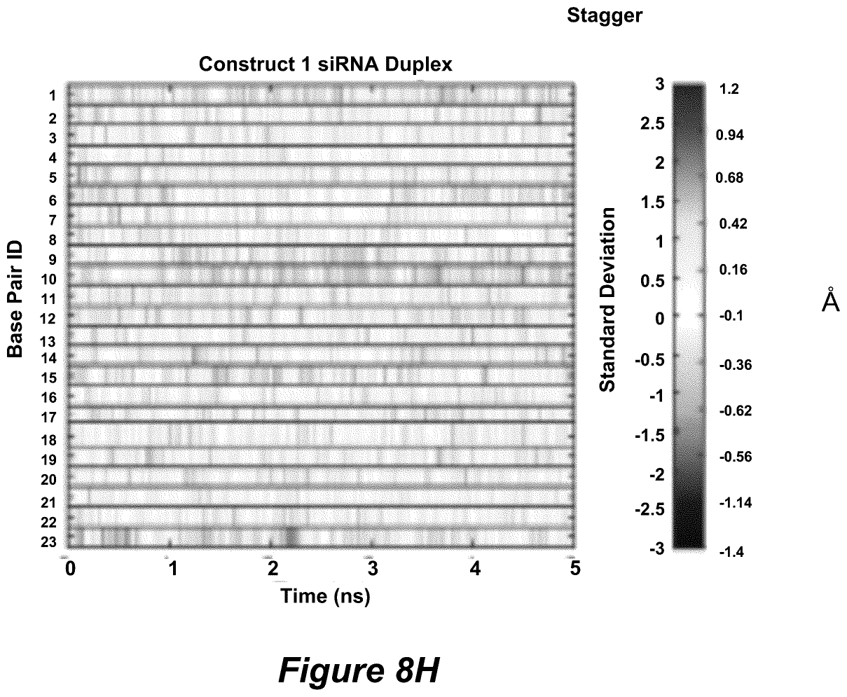

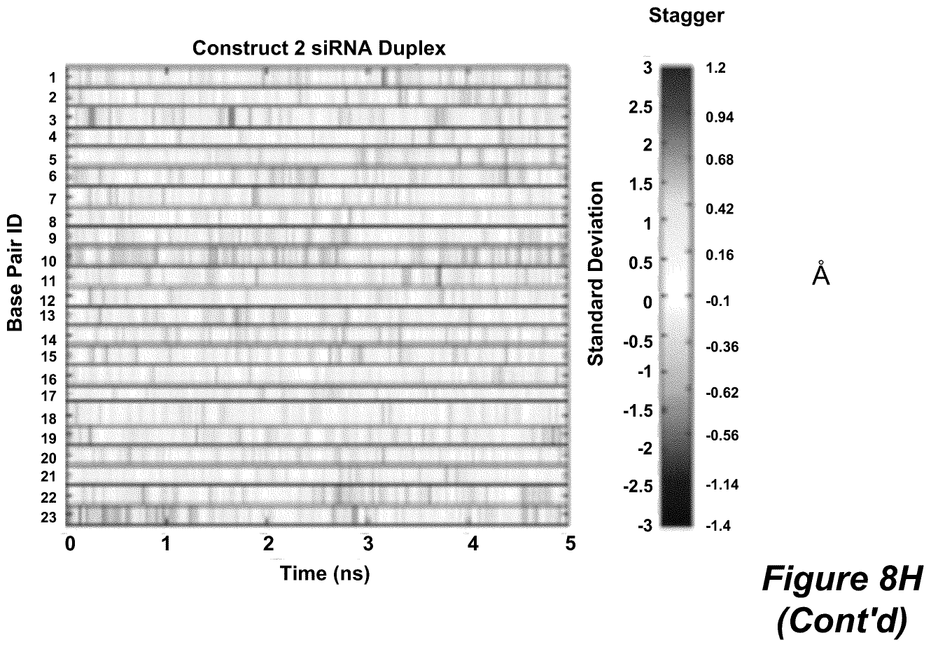

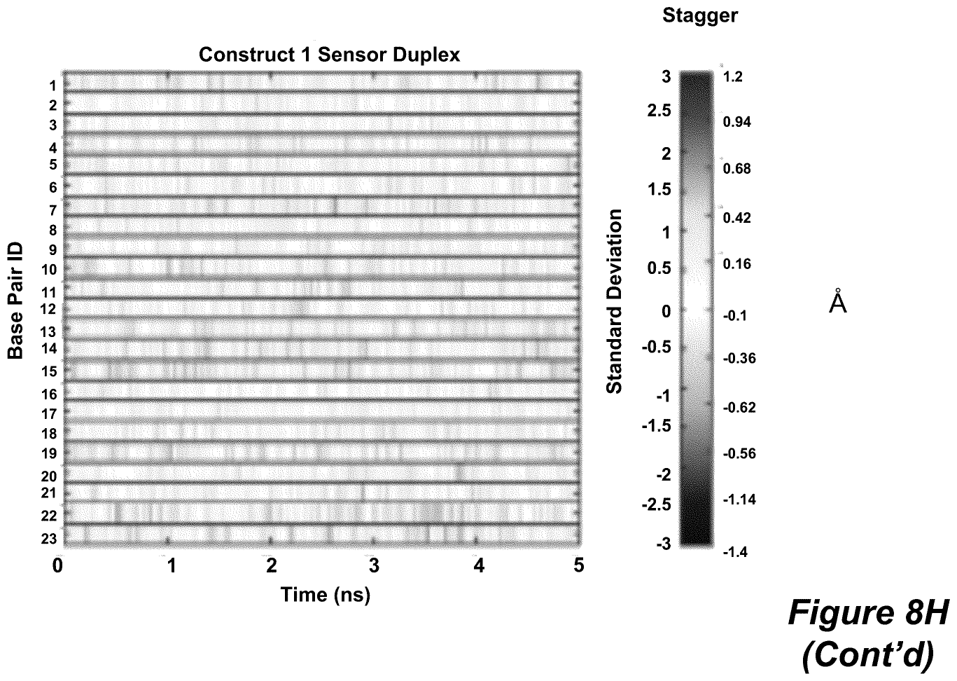

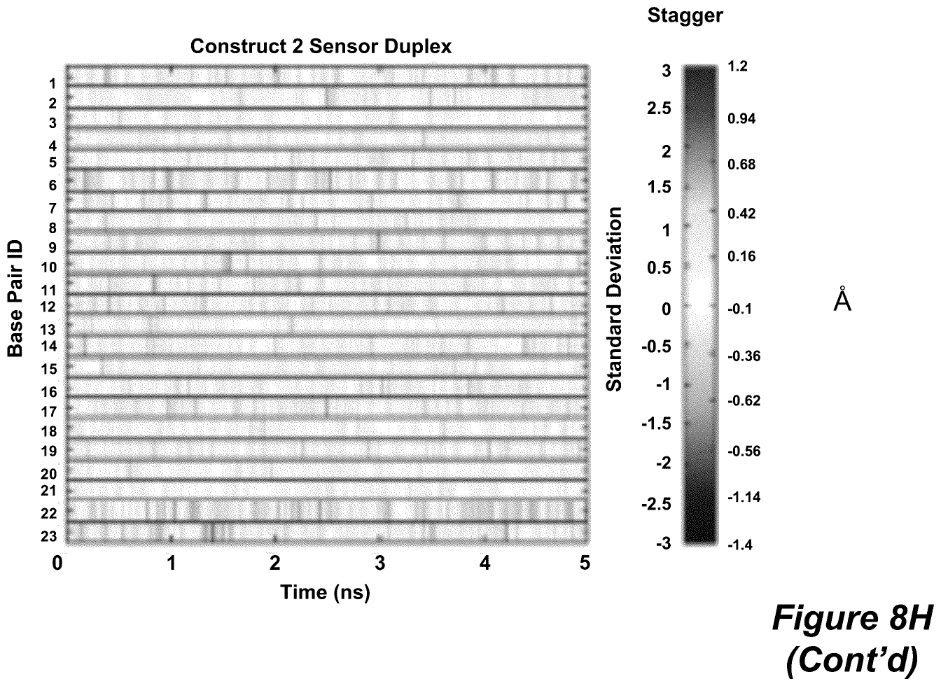

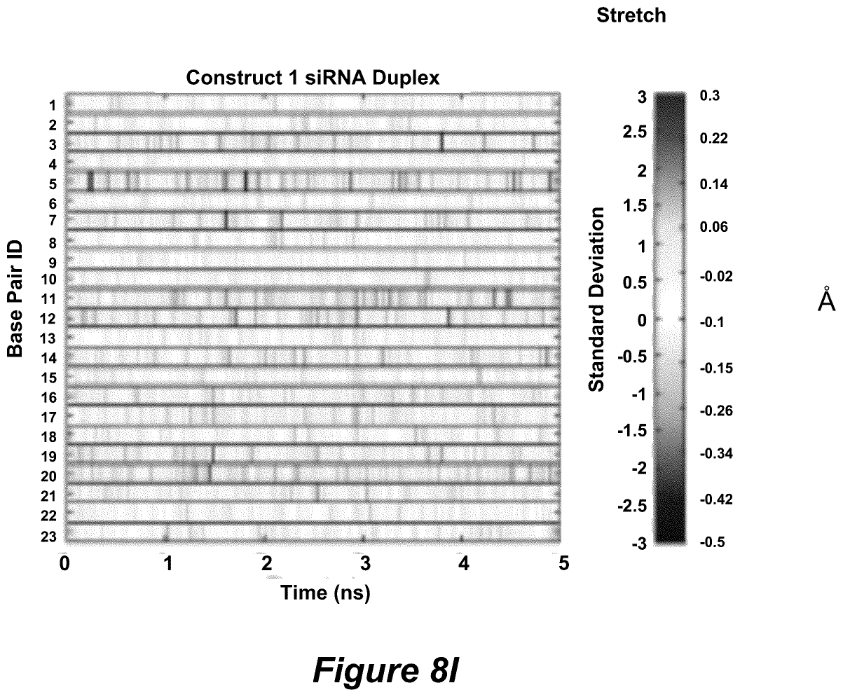

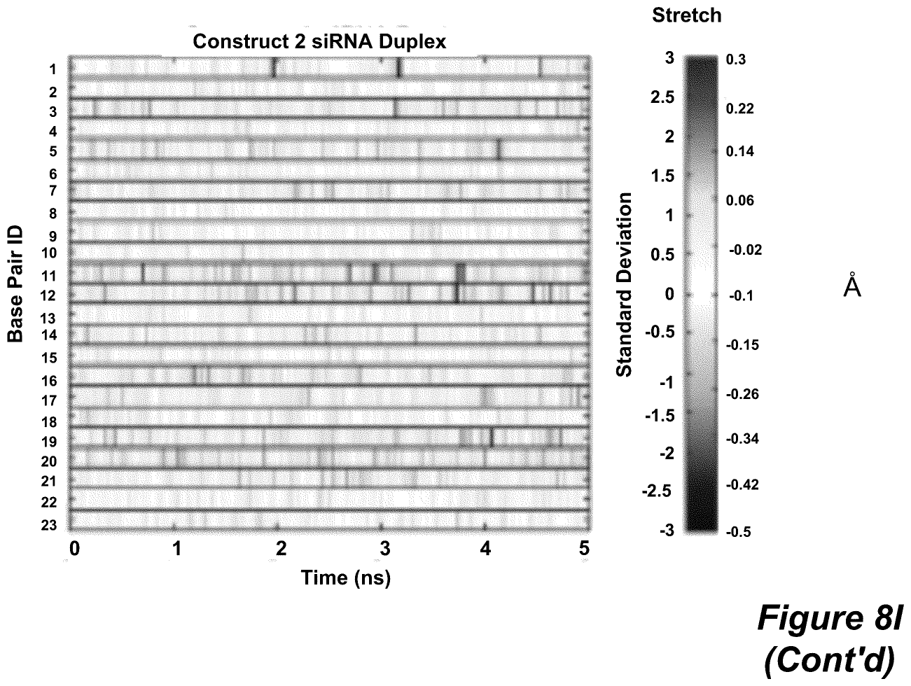

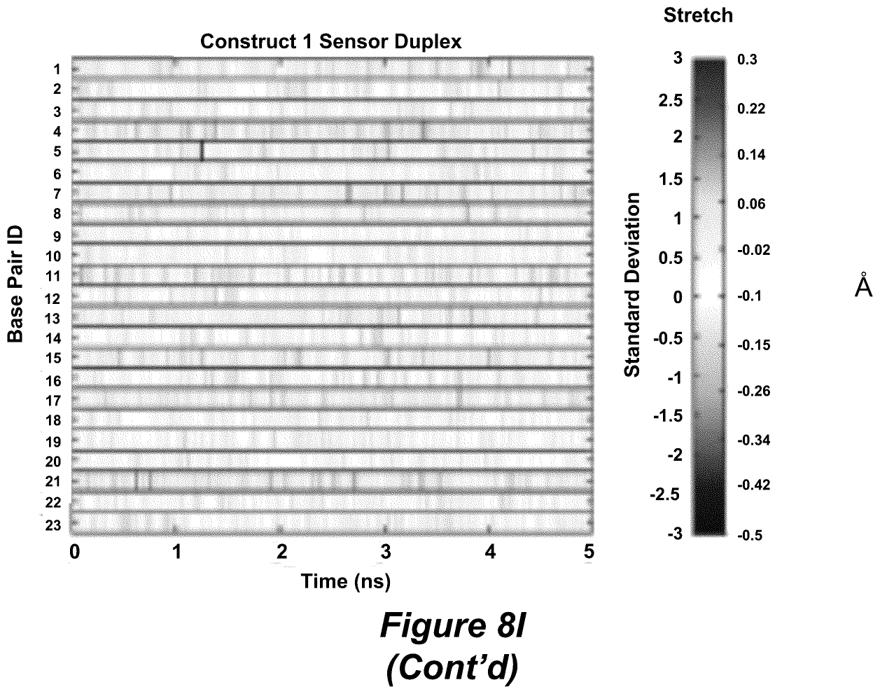

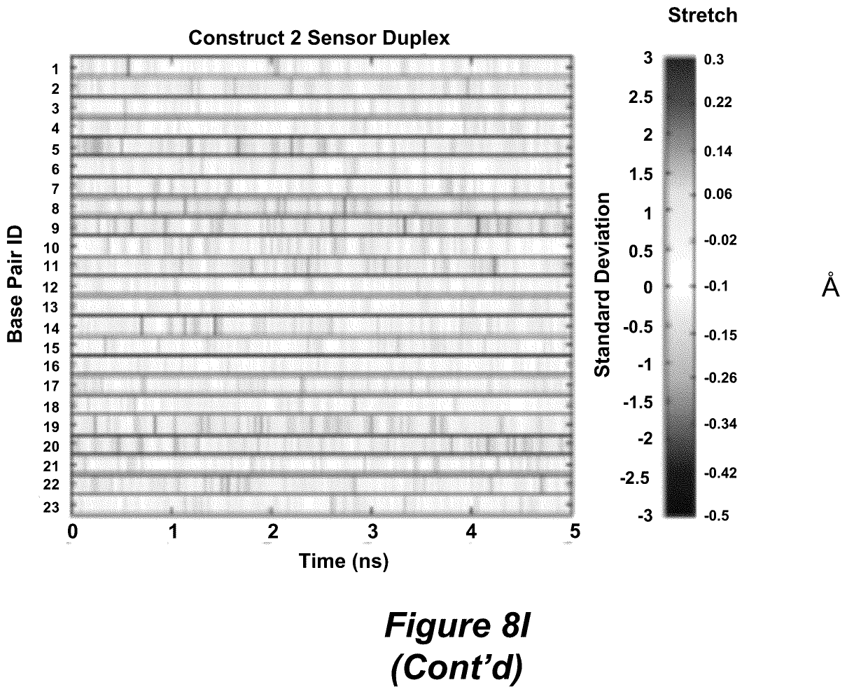

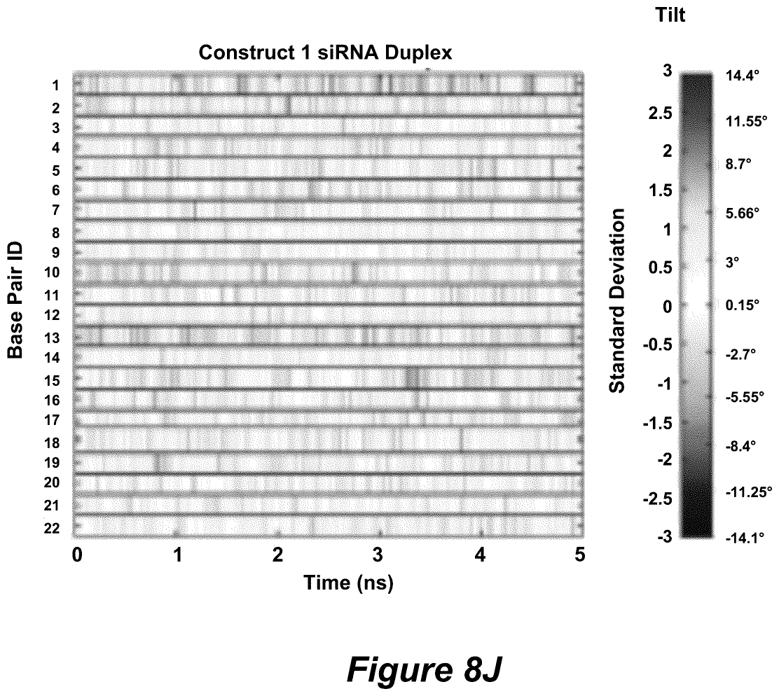

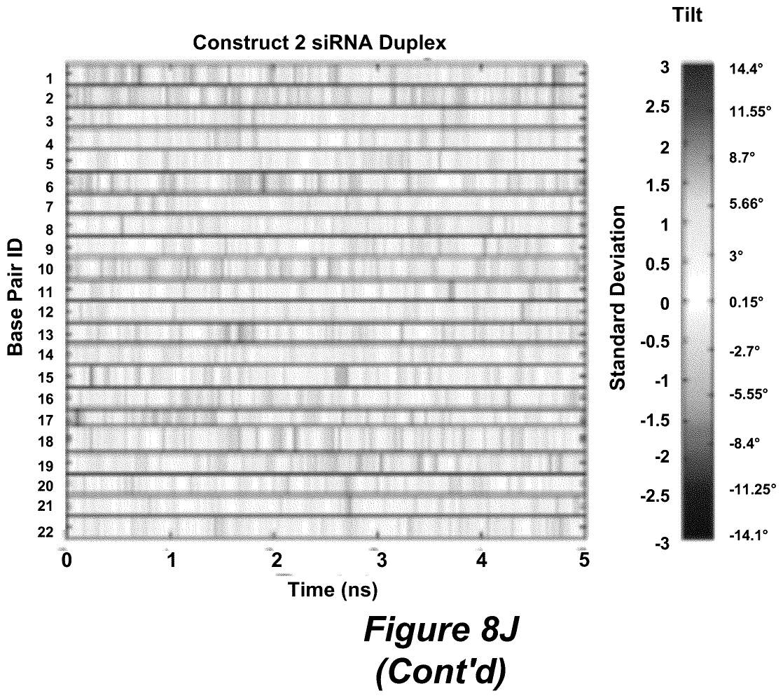

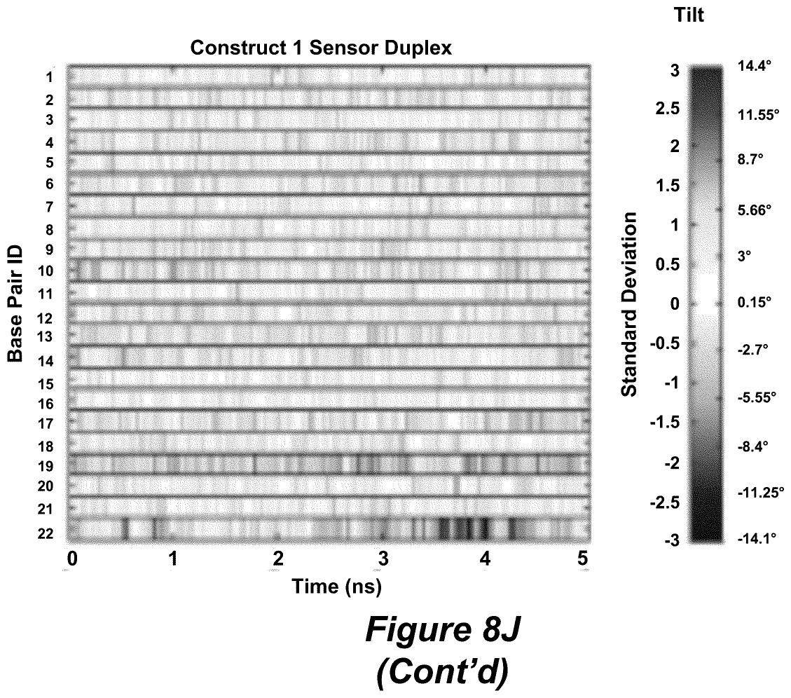

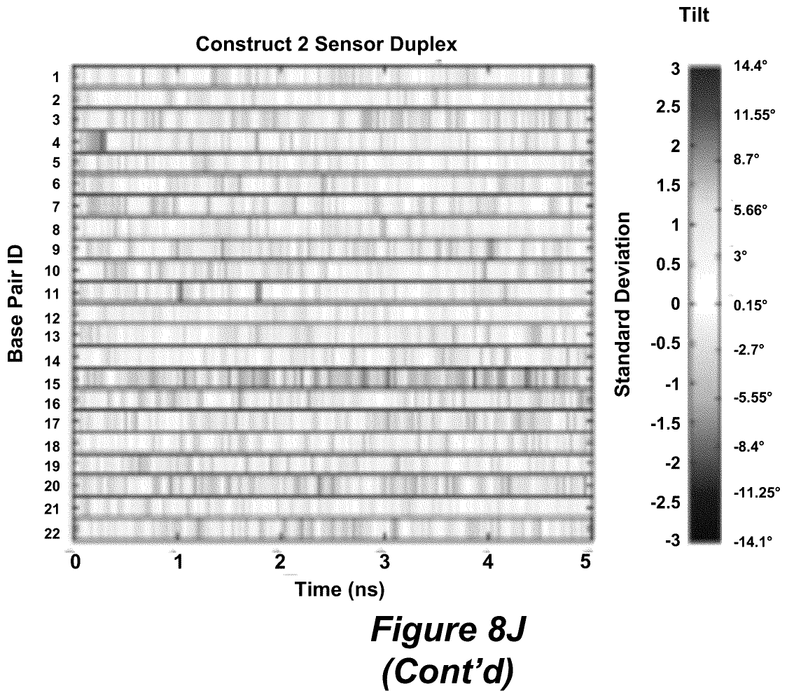

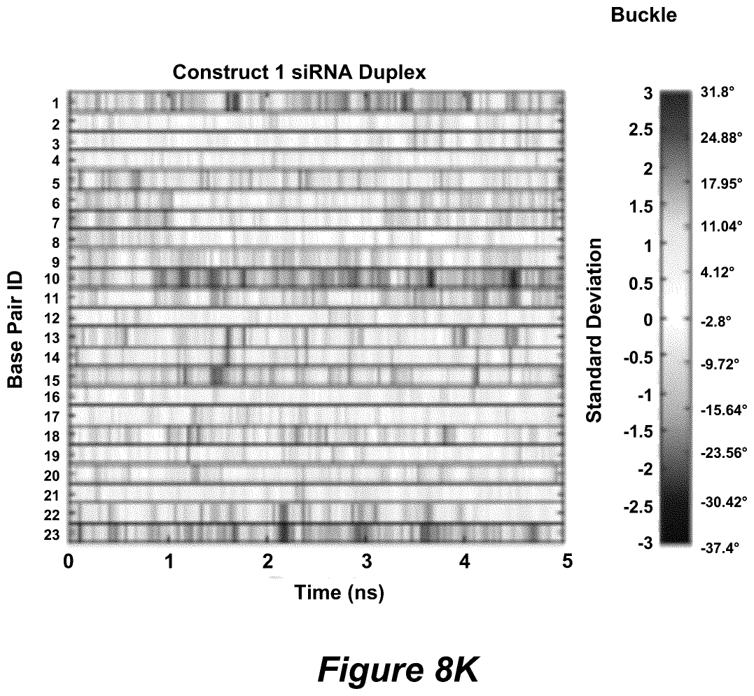

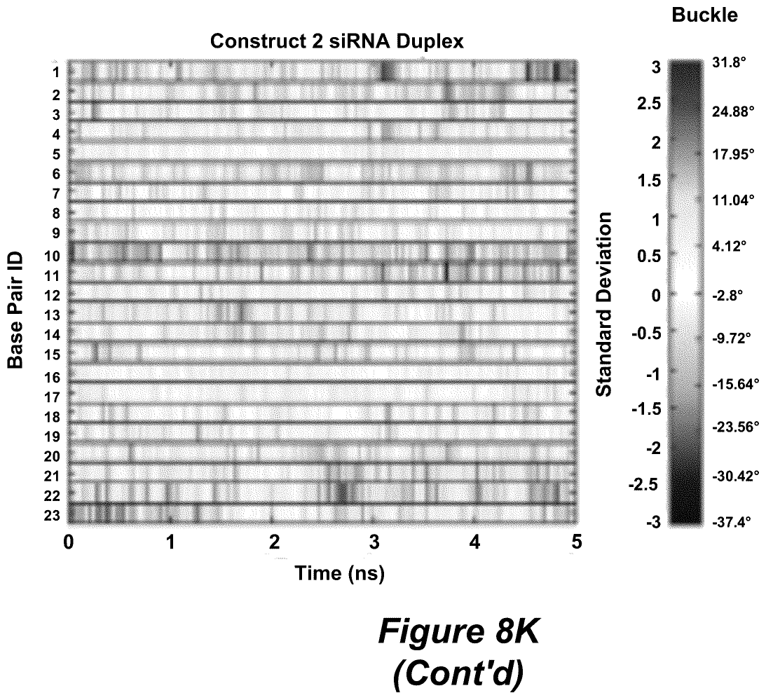

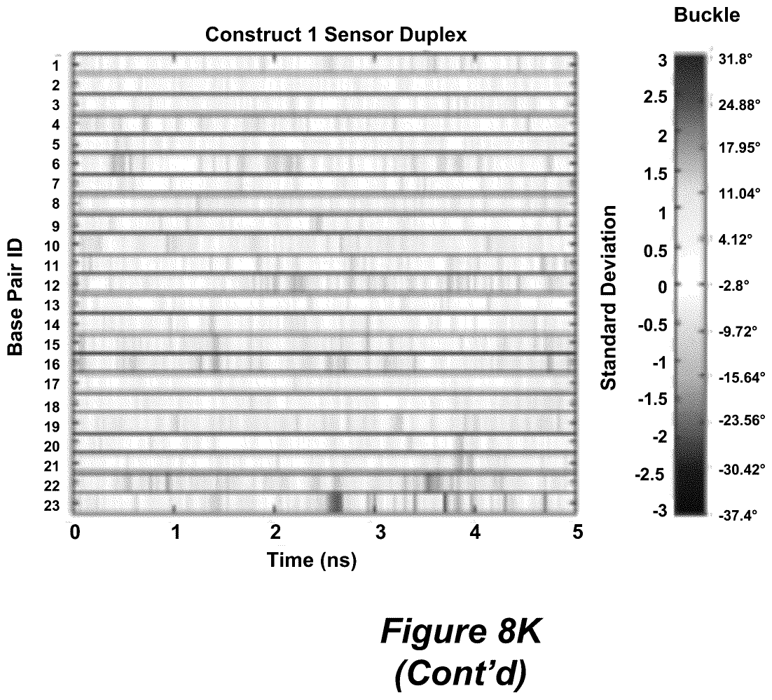

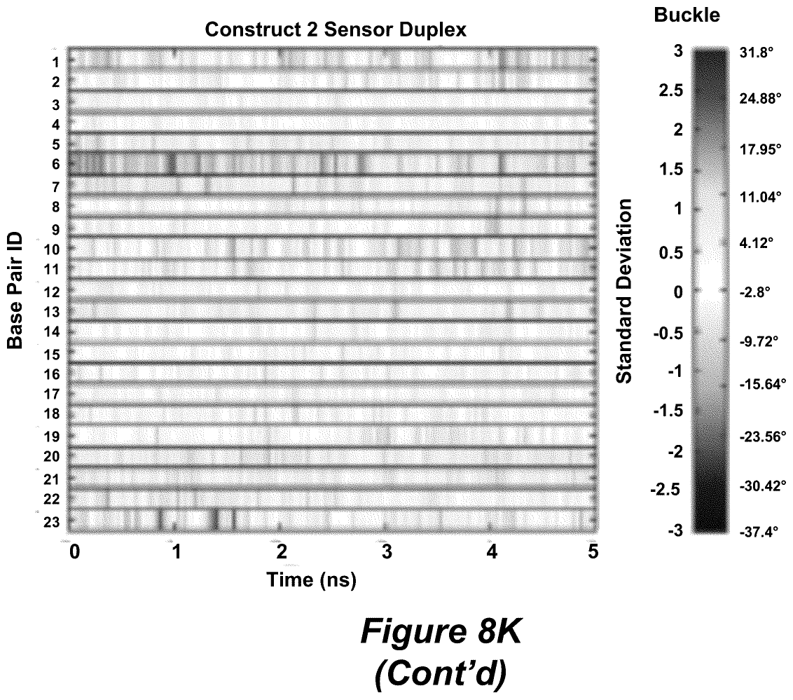

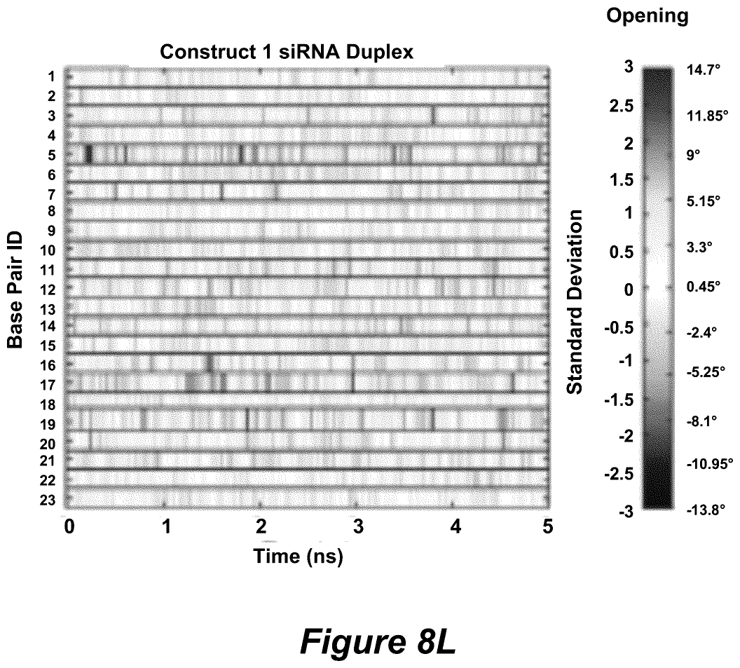

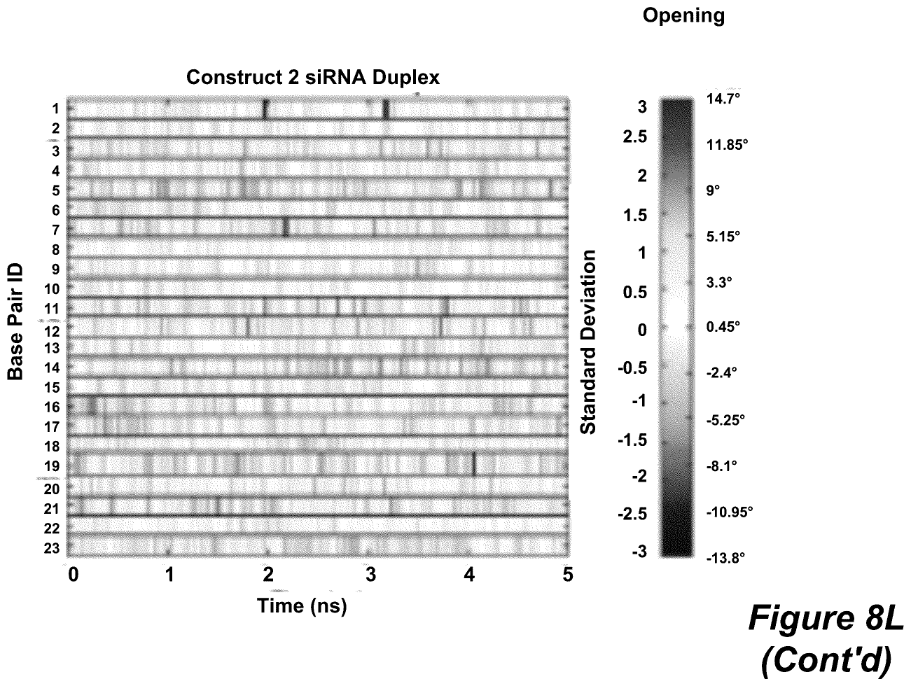

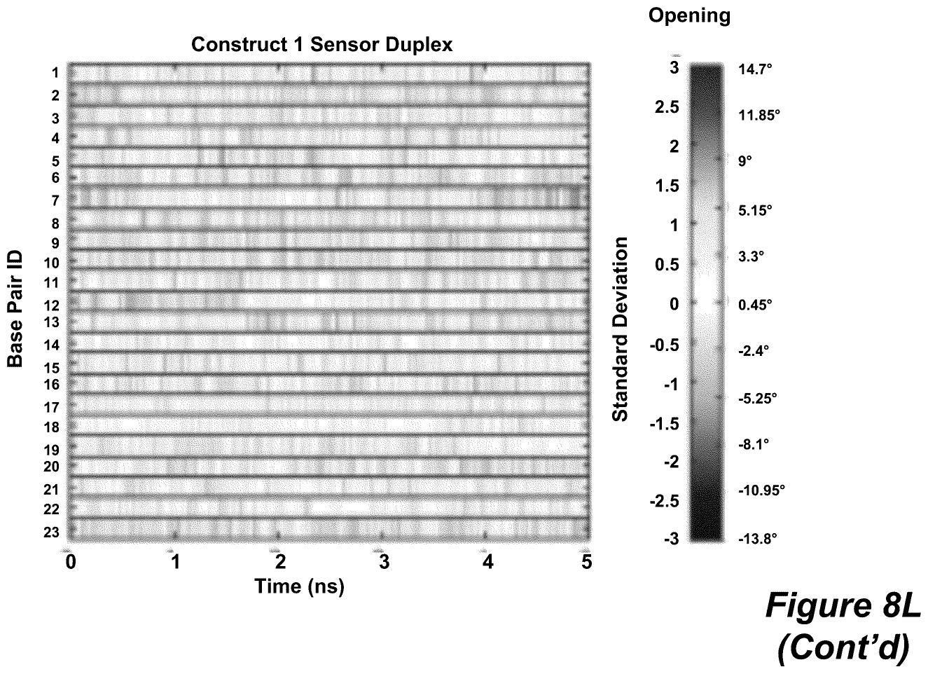

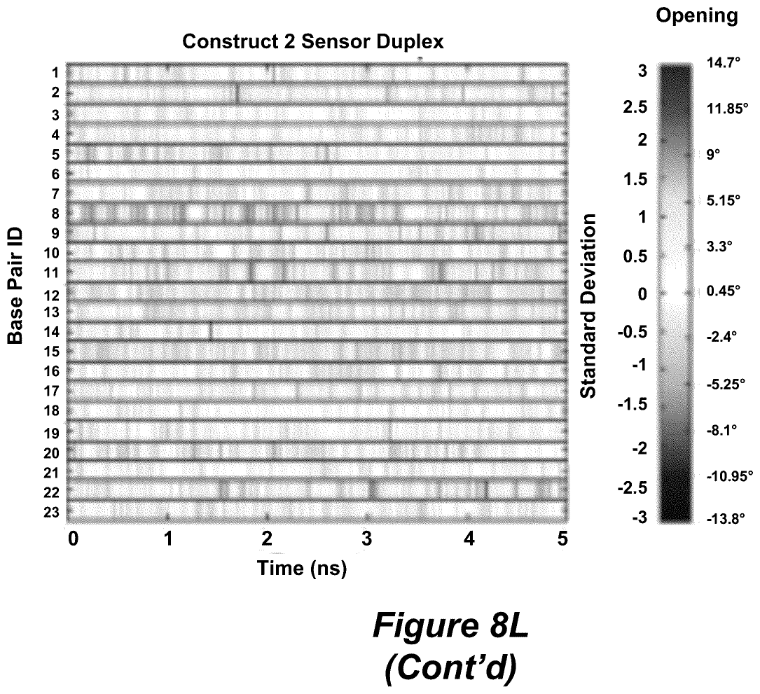

Further optimization of chemical modifications in key areas of the construct were performed (FIG. 3a, highlighted areas). The OFF and ON state RNAi activities of construct II variants were measured (FIGS. 7, 8, construct II.1-5) with differing patterns of chemical modifications at the 3′ and 5′ termini of the core strand (FIG. 3b, region A, flanking the central nick in the sensor duplex) and in the exonuclease-blocking domain of the RNAi duplex (FIG. 3a, region B).

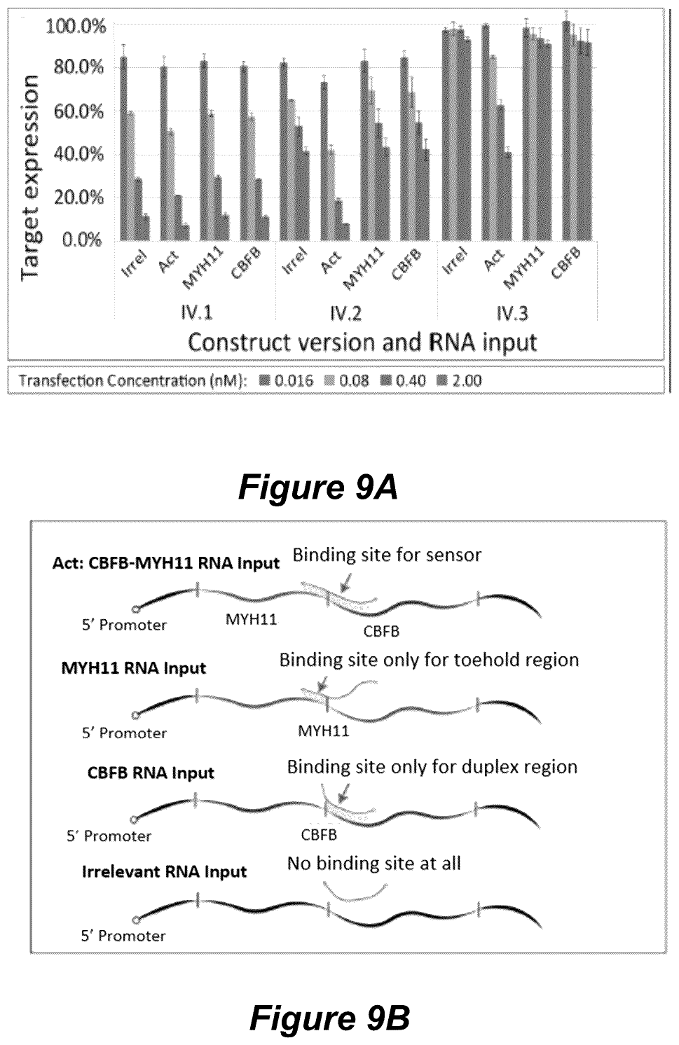

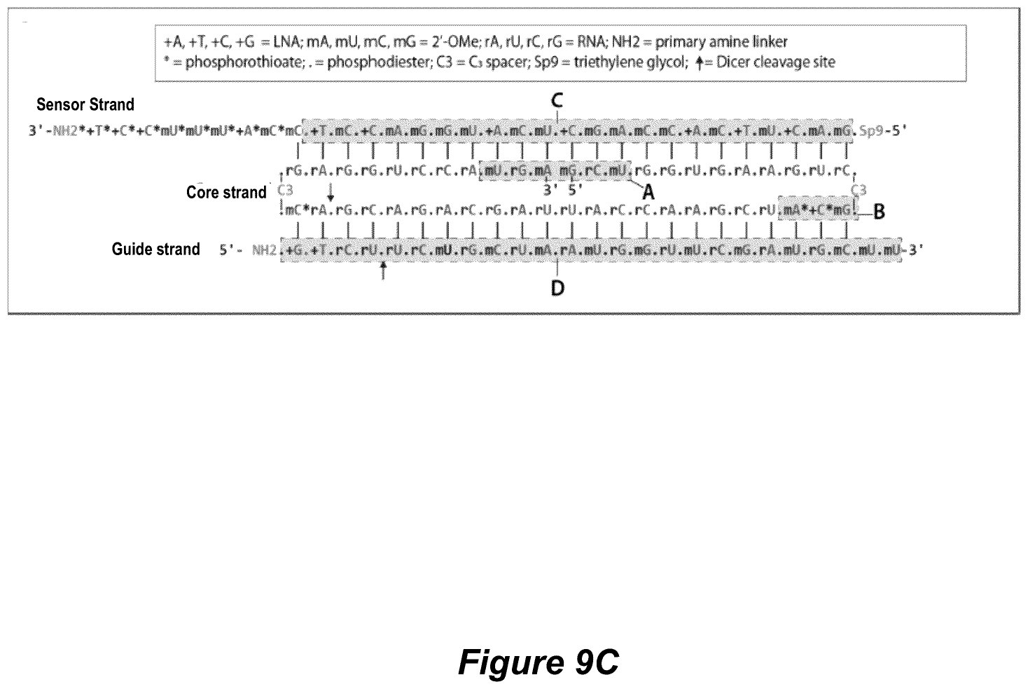

For region B, the results showed that that thermodynamic stability at the ends of the RNAi duplex may be important for suppression of OFF state RNAi activity. The replacement of a 2′-OMe modification in the center of the nuclease-blocking module with a locked nucleic acid (LNA) modification (FIG. 9c region C) reduced background RNAi activity without compromising RNAi potency (FIG. 5g, II.1 vs II.2).

For region A, to shut down OFF-state RNAi activity, the 3′ and 5′ termini of the core strand need to be protected by chemical modifications to shut down OFF-state RNAi activity. Background RNAi activity increased significantly when PS backbone linkages were replaced by phosphodiester linkages (FIG. 5g, II.2 had 3 consecutive terminal PS, II.3 had 2 alternating PS, II.4 had single terminal PS), and the ability to control RNAi activity was entirely lost with unmodified termini (II.0).

A problem with the use of PS linkages in region A is that each PS is a stereocenter with two possible enantiomeric conformations. Unfortunately, the more nuclease resistant Sp conformation is destabilizing to RNA base pairing³⁴. To improve thermodynamic stability of the sensor duplex and reduce racemic heterogeneity, PS backbone modifications were replaced with 2′-OMe base modifications (FIGS. 5h, 8c constructs II.5-7). The use of three consecutive (II.5), two alternating (II.6), or one terminal (II.7) 2′-OMe base at each core strand terminus all significantly reduced background RNAi activity. Furthermore, all three motifs increased ON state RNAi potency over PS modified analogues (FIG. 5h, II.5-7 vs II.2, ON state).

To determine whether 2′-OMe modifications allowed RNAi activation by cellular RNA transcripts, RNAi activation for II.1, II.2, II.6, and II.7 was compared (FIG. 5i) using a two-step transfection protocol. Both II.6 and II.7 showed significantly reduced background RNAi activity, rejected activation by mismatched RNA transcripts, and exhibited increased RNAi knockdown of the Renilla luciferase target in cells that expressed sequence matched inputs for the sensor (FIG. 5i, ˜50% reduction in target expression at 2 nM Cond-siRNA concentration).

To test the general applicability of region A optimizations, a version of construct III with 2′-OMe protected core strand termini was created (FIG. 8d, III.2) and its activation by cellular RNA transcripts was tested (FIG. 5j). Compared with the original PS protected variant (III.1), III.2 had significantly reduced background RNAi activity in cell expression mismatched inputs and better RNAi activation in cells expressing correct inputs (FIG. 5j, III.1 vs III.2). Notably, the removal of the terminal 2′-OMe modifications from just one of the core strand termini (III.3, the 3′ overhang of the core strand) nearly abolished control over RNAi activity (FIG. 5j, III.3).

Having optimized the core strand, the sensor strand is also improved. The initial constructs (I.1, II.1, III.1) used fully modified sensor strands with PS, LNA and 2′-OMe modifications in all regions. PS modification in the duplex region of the sensor may increase spurious activation by reducing base pairing stability. Also, other researchers have used 2′-OMe modifications alone without LNA to stabilize strand displacement switches for operation in mammalian cells^{8,19}. Disclosed herein a series of construct III variants that combined a 2′-OMe protected core strand (region A) with sensor strands that had either LNA+2′-OMe+PS (FIGS. 5k, 8e, III.2, III.4), LNA+2′-OMe (III.5), 2′-OMe only (III.6), or no modifications (III.7) in the duplex region of the sensor strand were tested. The results showed that the RNA (III.7) and 2′-OMe only (III.6) versions of the sensor strand were unable to suppress unintended RNAi activity (FIG. 5k). The most optimal variant was III.5, which had LNA and 2′-OMe modified bases, but no PS modifications in the duplex region. At 2 nM Cond-siRNA concentration, construct III.5 had only 20% background RNAi activity in cells expressing mismatched RNA inputs and achieved more than 90% knockdown of the Renilla target in cells expressing the correct RNA inputs (˜10× reduction in target expression between matched and mismatched). Statistically significant RNAi activation could be seen down to only 80 pM of Cond-siRNA.

Using insights gained from optimization of the CBFB-MYH11 sensor for construct III, construct IV was developed. Construct IV is a CBFB-MYH11 sensing Cond-siRNA with an RNAi domain targeting MCL-1, an endogenous apoptosis inhibitor that is crucial for the survival of AML cells³⁵ (but not the HCT116 cells used for dual luciferase experiments). Three versions of construct IV were tested for RNAi activation in response to transcripts with CBFB-MYH11, CBFB, or MYH11 sequences. To ensure that the sensor activates on the fusion sequence, the sensor strand (same as used in construct III) was designed to be complementary to the MYH11 sequence in its toehold forming region and complementary to the CBFB sequence in its duplex region (FIG. 9b). Three versions of construct IV were tested (FIG. 8f). IV.1 had modification patterns analogous to prototype construct III.1; construct IV.2 had optimized region A, B, and sensor strand motifs. IV.3 had additional 2′-O-methyl modifications in the guide strand using patterns previously found to have small impact on RNAi potency³⁶. Surprisingly, both IV.2 and IV.3 had significantly improved suppression of background RNAi activity and improved RNAi switching compared to IV.1 (FIG. 9c). IV.2 had background RNAi leading to ˜60% reduction in the Renilla readout target at 2 nM of Cond-siRNA but showed ˜90% reduction in Renilla in the fusion sequence expressing cells. IV.3 had almost no detectable background RNAi, but also lower activated RNAi activity (˜60% reduction in Renilla at 2 nM).

In some embodiments, the chemical modifications of the Cond-siRNA construct disclosed herein include one or more of the following:
A. The use of a sensor strand where the highlighted region C in FIG. 3a, “Regions for screening of chemical modifications” has one or more of the following features:
   a. 50% or less of the backbone positions are phosphorothioate (PS) connections.
   b. 50% or more of the bases are chemically modified to resist nuclease degradation or increase the melting temperature of the duplex (Tm)
   c. 100% of the bases are chemically modified to resist nuclease degradation and increase Tm
   d. About 10%-50% of the bases are locked nucleic acid (LNA) or other chemically modified base with 2′-4′ bridging modifications that substantively increase the Tm.
B. The 5′ and 3′ termini of the core strand have one or more of the following features:
   a. The terminal base on the 5′ is a 2′-F, 2′-O-methyl, or other modified base that resists nuclease cleavage
   b. The terminal base on the 3′ is a 2′-F, 2′-O-methyl, or other modified base that resists nuclease cleavage
   c. The three terminal bases on the 5′ have the pattern MRM, where M is a modified base (2′-O-methyl, 2′-F), and R is an RNA base
   d. The three terminal bases on the 3′ have the pattern MRM, where M is a modified base (2′-O-methyl, 2′-F), and R is an RNA base
   e. The three terminal bases on the 3′ and 5′ do not have consecutive PS backbone modifications
   f. The portions of the core strand that are base-paired with the sensor strand have an alternating chemical modification pattern (MR)n
   g. The above where M is a chemically modified base that does not decrease Tm of the duplex when compared to the equivalent RNA base
   h. Any combination where both the 5′ and the 3′ ends of the core strand have at least one of the features from a-g
   i. The 3′ and 5′ regions of the core strand that are base-paired with the sensor strand are:
      (a) Entirely made out of the pattern (M)n, where M is a 2′-O-methyl or 2′-F modified base, or
      (b) at least 50% of the bases in this area are 2′-O-methyl or 2′ F and, up to 30%, 50%, 80% or 100% of the backbone connections are not phosphorothioate.
C. A core strand where the three bases base-paired with 3′ terminus of the guide strand have one or more of the following features:
   a. A M*+*M pattern, where M is a 2′ modified base (e.g. 2′-O-methyl or 2′-F), * is a PS backbone connection, and + is an LNA base or other 2′-4′ bridged base
   b. A M*+*+ pattern, as defined above
   c. A +*+*+ pattern
   d. A R*+*M pattern, where R is an RNA base
   e. A R*+*+ pattern
   f. A +*M*M pattern
   g. The patterns in a-f, where * can be either a PS backbone connection or an unmodified (phosphodiester) backbone connection
D. A guide strand with one or more of the following features:
   a. 30% to 95% of the bases are chemically modified bases (2′-O-methyl, 2′-F, LNA, 2′-4′ bridged bases)
   b. Where the two terminal bases on the 5′ are chemically modified
   c. Where the two terminal bases on the 5′ have at least one LNA
   d. Where the two terminal bases on the 5′ are connected by a PS backbone
   e. Where the two terminal bases on the 3′ are both chemically modified
   f. Where ˜5% to 50% of backbone connections are PS
   g. Where the bases flanking the indicated site of Dicer cleavage are not chemically modified.

In sum, disclosed herein is a programmable, conditionally activated small interfering RNA (Cond-siRNA). This device outputs an active RNA interference trigger against a target gene only upon detection of RNA transcripts from a different input gene. The improved performance of strand displacement sensors and realization of conditionally activated RNAi are attributable in part to five key design principles.

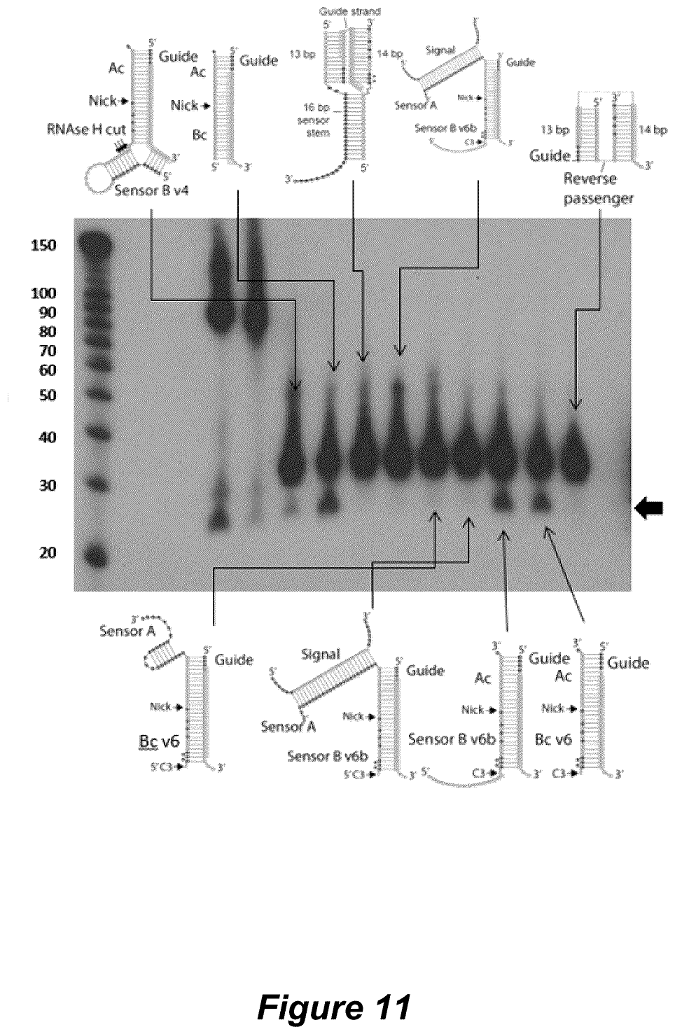

First, the RNAi trigger needs to completely dissociate from the waste duplex formed by the input RNA and the sensor strand in order to have potent RNAi activity. Prior schemes for conditional RNAi often featured activated RNAi triggers that remain attached to the input signal (e.g. an mRNA) via Watson-Crick base pairing^{15,17,18,37,60}. During development of a prior generation conditional RNAi trigger, it was found that the connection of Dicer substrates to adjacent 2′-O-methyl modified duplex RNA domains significantly reduced RNAi activity (FIG. 11). A possible cause for this could be binding of a Dicer inhibiting protein such as PACT³⁸ to the extended duplex. However, dissociation of the RNAi duplex from the input-sensor duplex was necessary for achieving simultaneous optimization of OFF state RNAi suppression and ON state RNAi potency.

The sequences used for the constructs in FIG. 11 are as follows:

TABLE 4
Sequences for Testing Constructs

| | | SEQ |
|---|---|---|
| | | ID NO |
| Guide | mCmUmUmGCGUCUGAGGGAUCUCUAGUUACCUU | 93 |
| DNA probe | dAdAdGdGdTdAdAdCdTdAdGdAdGdAdTdCdCdCdTdCd | 94 |
| for guide | AdGdA | |
| strand | | |
| Sensor A | CCUCAGACGCAAGmCmUmGmAmUmGmAmGmCmUmCm | 95 |
| | UmUmCmGmUmCmG | |
| | *mC*mU*mG*mU*mC*mU*mC(18s)(idT) | |
| Ac | CCUCAGACGCAAG(idT) | 96 |
| Sensor B v6b | CGACGAAGAGCUCAUC(c3)mG*mG*mUAACmUAmGAmGA | 97 |
| | UmC | |
| Sensor B v4 | mAmAmGmGmUdCdCdCdTdGdAdTCGACGAAGAGCUCAU | 98 |
| | CAGGGUAAC mUAmGAmGAUmC | |
| Bc | GGUAACUAGAGAUC | 99 |
| Bc v6 | (c3)mG*mG*mUAACmUAmGAmGAUmC | 100 |
| Signal | mAmAmAmAmAAGCGGAGACAGCGACGAAGAGCTCATCA | 101 |
| | GmAmAmAmAmA mA | |
| Reverse | CCUCAGACGCAAGGGUAACmUAmGAmGAUmC | 102 |
| Passenger | | |

Second, past designs for conditional RNAi triggers and other DNA circuits have featured either single-construct^{15,16} or multi-construct¹⁷ schemes for translating an input sequence into an independent output sequence. Single-construct translators should theoretically be intrinsically more efficient in signal detecting and transduction. However, the disadvantage is that the RNAi trigger must be concealed within the construct, creating an opportunity for spurious RNAi activation due to construct degradation. The success of the disclosed Cond-siRNA design indicates that the advantages of single-construct translators can be utilized while the risk of spurious activation effectively controlled.

Third, extensive chemical modifications are key to proper functioning of strand displacement sensors, and by extension, the Cond-siRNA. The working examples of the optimization experiments show that: 1) the duplex domain of the sensor strand needs to have both LNA and 2′-O-methyl modifications but not PS modifications; 2) the termini of the protecting strand need to be modified with either PS or 2′-O-methyl; 3) thermodynamically stabilizing modifications generally improve suppression of background activation, while thermodynamically destabilizing modifications such as PS backbones can actually increase spurious activity when used extensively in duplex regions; 4) while the chemical modifications were not tested in the toehold domain, the combination of LNA, 2′-O-methyl and PS modifications are beneficial, as they improve the base-pairing affinity and nuclease resistance of the single stranded overhang.

Fourth, the endogenous RNA degradation machinery can be an effective tool for construct switching. As shown in the working examples, the chemically modified 3′ and 5′ terminal regions of the core strand were highly stable when base-paired with the sensor strand but vulnerable to degradation when unpaired. This differential susceptibility to nuclease activity can be accomplished with PS modified termini but appeared more effective with 2′-OMe modifications. As demonstrated herein, the degradation of single stranded overhangs can be stopped at the ends of duplexes by exonuclease blocking domains. The trimming of the disclosed RNAi duplex using this scheme resulted in a potent RNAi trigger from the DX secondary structure. Similar schemes might be useful in the dynamic reconfiguration of other nucleic acid nanostructures.

Finally, the separation of the sensor and RNAi domains in two distinct duplexes was important for intracellular stability, programmability and ease of engineering development. First, there is no base-pairing overlap between the two domains and no competing secondary structure conformation. This ensured a large margin of thermodynamic stability and simplified programming for new input and output sequences. Second, the dimensions of the duplexes and the linkage chemistry at the crossover points were configured to minimize strain in the tertiary structure (FIG. 1c). This further enhanced the thermodynamic stability. Third, since the duplexes did not overlap, chemical modifications can be applied to the sensor domain without compromising compatibility of the RNAi domain.

The following examples are provided to better illustrate the claimed invention and are not to be interpreted as limiting the scope of the invention. To the extent that specific materials are mentioned, it is merely for purposes of illustration and is not intended to limit the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Construct Design

Cond-siRNAs were designed for specific pairing of inputs and targets using an iterative protocol. A suitable 21-nt guide strand sequence for the RNAi domain was obtained from previously validated siRNAs, literature sources, or siRNA design tools. A 23-bp Dicer substrate was created from the chosen guide strand by adding four G/C rich bases to the 5′ of the guide strand. Nupack (RNA strand, parameters disclosed by Mathews⁵⁹, some dangle treatment) was used to confirm that the RNAi duplex forms with >95% probability at 1 nM of guide (antisense) and sense strands.

From the sequence of the input biomarker, a list of all possible 31 to 33 nt sensor segments (antisense to the input) was generated. For the CBFB-MYH11 fusion sequence, only sensor segments that approximately met parameters illustrated in FIG. 9b were considered. Sensor sequences were ranked for uniqueness in the transcriptome of the target animal using NCBI BLAST. For human cancer cell lines, sequences were checked against human transcript+genomic collection using the BLASTn algorithm. Where possible, sensor segments that have more than 17 bases of sequence complementarity and complete overhang complementarity to known or predicted RNA transcripts were eliminated.

Starting with the most unique sensor segments, construct core strand sequences in accordance with desired structural parameters for the Cond-siRNA were selected. For example, core strands had sequences of the form 5′-B-C3-P-C3-A-3′ where A and B are complementary to the 5′ and 3′ ends of the sensor strand's putative duplex domain, P is complementary to the putative guide strand, and C3 are C3 linkers. Nupack was used to rank the thermodynamic stability of the duplexes formed between sensor strand segments and their corresponding 5′ and 3′ core strand overhangs. RNA strand having parameters disclosed by Mathews et al.⁵⁹ was used with some dangle treatment. Ideally, >95% of strands should be base-paired at 1 nM strand concentration. The core strand was also checked to confirm that it did not have heavy internal secondary structure.

The best constructs having the guide strand, core strand and sensor strand sequences designed above were chosen and subjected to chemical modifications. Exiqon's oligonucleotide design tools (www.exiqon.com/oligo-tools) were used to optimize the placement of LNA modifications. LNA modifications were added to the sensor strand approximately 1 per every 3 to 4 bases. The LNA Oligo Optimizer tool was used to check that the LNA pattern did not lead to secondary structure or self-pairing interactions with scores higher than 60. Self-complementarity and self-pairing scores were optimized to the extent possible.

### Example 2: Synthesis of Strands

Strands with LNA bases were synthesized by Exiqon Inc (now a division of Qiagen). Strands without LNA were synthesized by GE Life Sciences Dharmacon (now a division of Horizon Discovery Group). All strands were ordered with PAGE or HPLC purification, according to recommendations by the manufacturer.

### Example 3: Assembly of Cond-siRNAs

Cond-siRNAs were assembled by thermal annealing in 1× phosphate buffered saline (PBS). Constructs can be assembled with or without purification. Assembly quality can be assessed using non-denaturing gel electrophoresis on 10% to 15% PAGE in 1× TBE at 4° C.

For assembly without purification, sensor, core and guide strands were mixed at a 1.1 to 1.00 to 1.1 molar ratio at 50 nM or 100 nM concentration in 1×PBS PH ˜7.0. Using a slight excess of sensor and guide strands helped to prevent production of constitutively RNAi active guide+core duplexes. A PCR thermocycler with the following program was used:
Heat lid to 105° C.
Hold at 85° C. for 30 seconds to denature strand
Cool to 50° C. at a rate of 0.1° C./second.
Hold for 45 min at 50° C.
Cool to 37° C. at a rate of 0.02° C./second
Cool to 4° C. at maximum rate and hold.

For assembly with purification, the sensor, core, and guide strands were mixed and assembled at 1 uM nominal concentration in 1×PBS using the above annealing protocol. Assembled constructs were then loaded on Bio-Rad mini protean 10% native PAGE gels in TBE buffer, and run at 125V at 4° C. for ˜45 min. The bands corresponding to the Cond-siRNA were visualized and excised under UV lamp illumination.

The excised bands were extracted by electro-elution using a Harvard Apparatus Electroprep system according to the manufacturer's instructions. Gel pieces were placed in a 0.5 mL chamber sealed by a 100K MWCO filter membrane and a 2K MWCO filter membrane. Constructs were eluted through the 100K MWCO membrane and trapped in an adjacent 0.5 mL chamber formed by the 100K MWCO membrane and a second 2K MWCO membrane. Elution occurred in 0.1 M Na₂HOP₄ buffer (˜pH 7.0) at 4° C. for ˜45 min. The power supply was set to maintain a constant current of 15 mA with a voltage cutoff of 65 V.

Concentrations of purified constructs were calculated by comparison with Cond-siRNA standards at a known concentration using SYBR Gold staining on non-denaturing PAGE with quantitation using a Bio-Rad ChemiDoc XRS+ Imager.

Assembled constructs are best used immediately after assembly or purification. Constructs can also be stored in aliquots at −80° C. indefinitely. However, the freeze thaw cycles compromised construct quality and resulted in construct disassembly. Disassembled constructs can be re-assembled by repeating the thermal annealing immediately prior to assay.

The unpurified constructs were used in various tests because assembly yield was already high and purification did not consistently improve construct performance.

### Example 4: Strand Displacement Assay

Pre-assembled constructs were prepared at 50 nM nominal concentration and combined 1:1 with 50 nM oligonucleotide activators (or PBS for control) at 37° C. in PBS buffer to obtain mixtures with 25 nM input signals and constructs. Construct-input combinations were then incubated in a PCR thermocycler at a constant 37° C. over 4 hours. Samples were collected at the indicated time points and immediately frozen at −80° C. in 1× native PAGE loading dye. At the end of the experiment, all samples were rapidly thawed, and analyzed using non-denaturing PAGE.

### Example 5: Generation of Dual Luciferase Reporter and Activator Plasmids

All clones were generated using standard molecular biology protocols by annealing DNA oligos for the specific insert followed by ligation into the indicated sites of the parental vector. The accuracy of all constructs was verified by DNA sequencing.

### Example 6: PsiCHECK Dual Luciferase Reporters

The indicated DNA oligos below were annealed and ligated into the XhoI and NotI sites of a psiCHECK 2 (Promega) dual Luciferase reporter. Nucleotides in bold font constitute the sense target sequence. Lower case nucleotides indicate restriction site 5′ overhangs.

HIV U5 region target
(SEQ ID NO: 103)
5′- tcgaGTCTGGTAACTAGAGATCCCTCAGACCC
(SEQ ID NO: 104)
5′- ggccGGGTCTGAGGGATCTCTAGTTACCAGAC
MCL-1 target
(SEQ ID NO: 105)
5′- tcgaGCTGCATCGAACCATTAGCAGAAA
(SEQ ID NO: 106)
5′- ggccTTTCTGCTAATGGTTCGATGCAGC

### Example 7: RNA Input Transcripts for Signal Activation Experiments

The activator sequences were expressed as part of a chimeric tRNA transcript. The first part consists of a modified⁵ tRNA^{Lys3} with 3′ terminal CCA, mature sequence shown in its entirety below. The CCA prevents endonucleolytic cleavage by the pre-tRNA processing enzyme, tRNAse Z. tRNA Pol III promoters were internal and contained within the coding sequence of the tRNA DNA.

For cloning a parental plasmid containing the first 69 nucleotides of tRNA^{Lys3}, terminating in an NruI restriction site⁴⁸ was used. Digestion of the parental plasmid with NruI generates a blunt end immediately following nucleotide tRNA 69. Annealed overlapping oligos encode the remaining modified tRNA nucleotides followed by the specified activation sequence. Each activation sequence terminates in a 12-base tetraloop (GGCGCAAGCC) (SEQ ID NO: 107) followed by a T6 run encoding the Pol III terminating sequence U4+RNA transcript sequences are listed below.

For constructs I and II, tRNA^{Lys3} leader sequence, 5′->3′: GCCCGGAUAGCUCGGUCGGUGGAGCAUCAGACUUUUAAUCUGAGGGUCCAGG GUUCGAGUCCCUGUUCGUGCACCA (SEQ ID NO: 108)—Activator sequence. The sequence in bold is the binding site for the Northern blot probe. The activator sequences are listed in Table 1 and Table 2. Table 5 below lists the Northern blot probes.

TABLE 5
Northern Blot Probes

| | |
|---|---|
| tRNA probe | CTGGACCCTCAGATTAAAAGTC |
| | (SEQ ID NO: 109) |
| CBFβ probe | CTCCATTTCCTCCCGATGAGACCTGTC |
| | (SEQ ID NO: 110) |
| MYH11 probe | CGCTTGGACTTCTCCAGCTCATGGAC |
| | (SEQ ID NO: 111) |
| U5 guide | AAAGGTAACTAGAGATCCCTCAGA |
| strand probe | (SEQ ID NO: 112) |

### Example 8: Tissue Culture

All analyses utilized HCT 116 colorectal carcinoma cells. Cells were maintained using McCoy's 5A basal medium (Irvine Scientific, USA) supplemented with 10% fetal bovine serum (FBS), 1.5 mM L-glutamine (Irvine Scientific, USA) and 10 mM pyruvate (Irvine Scientific, USA) without antibiotics and kept in a humidified 5% CO₂ incubator at 37° C.

### Example 9: Northern Blot Analysis

Analysis of activator expression was performed in 6-well plates using: 2 μg of plasmid DNA in 250 μL OptiMEM and 250 μL 1:50 diluted Lipofectamine 2000. Liposomes were allowed to form according to the manufacturer's instructions and added to cells with 2 mL fresh full medium. Medium was replaced at 18 hours, and depending on the length of transfection, at least once each subsequent day and 6 hours prior to RNA harvest. Analysis of OFF and pre-activated (ON) c-siRNAs were performed similarly; however, the indicated amount of RNAi complex was added to 2 μg of pBluescript plasmid as carrier in 250 μL OptiMEM.

Total RNA was harvested using 1000 μL RNA Stat-60 (Tel-Test, Inc) and processed according to the manufacturer's instructions with the addition of a second organic extraction using 1:1 phenol: chloroform extraction prior to precipitation; RNA pellets were washed twice with 70% ethanol prior to evaporation of excess ethanol and re-suspension in RNAse-free TE, pH 6.8.

For Northern analysis, 15 μg of total RNA were run on 8% (for activators) or 12% (for c-siRNAs) urea-PAGE gels (15 cm) with 32P-labeled Ambion Decade markers. Gels were electro-blotted to Hybond XL (GE Healthcare Life Sciences), pre-hybridized and hybridized at 37° C. using Sigma Perfect Hyb Plus and hybridized with 5-10 picomoles of P32-5′ end-labeled oligo probe. Blots were washed at 37° C. with 4-5 changes of 2×SSC/1% SDS. With serial hybridizations, old oligo probe was removed from the membrane according to manufacturer's instructions and checked by re-exposure prior to re-hybridization unless otherwise indicated. Hybridization of U6 snRNA was used a loading control. Cloning procedures, oligos and all probe sequences are listed in the tables.

### Example 10: Dual Luciferase Assays

Dual luciferase assays were performed using Promega Dual-Luciferase Reporter Assay System according to the manufacturer's instructions. The RNAi target sequence was cloned into the 3′ UTR of the Renilla luciferase gene on a psiCHECK-2 (Promega) vector, and Firefly luciferase was used as a reference control.

Cells were incubated and transfected in 48-well cluster plates. Cells were seeded one day prior to transfections and transfected at 50% confluency. Each experiment was repeated in its entirety at least three times to obtain biological replicates. A single-step transfection protocol for FIGS. 5b-5h, and a two-step transfection protocol for FIGS. 5i-5k and FIG. 9a.

### Single Step Co-Transfection Protocol:

For each experiment, a master mix of the psiCHECK (Promega Corporation) reporter plasmid in Opti-MEM (Thermo Fisher Scientific) was created. This master mix was separated into aliquots and either the pBluescript (Agilent) control or one of the activator plasmids added. The new mixtures were then divided yet again for addition of Cond-siRNA complexes at varying concentrations. Finally, a 1:50 dilution of Lipofectamine 2000 (L2K) was added at 1:1 volume ratio to the plasmid+Cond-siRNA mixtures to achieve the manufacturer recommended dilution of 1:100 L2K and incubated at room temperature according to the manufacturer's recommendation.

For each experimental condition (combination of activator and c-siRNA at a specific concentration), enough mixture (3.3× amount needed) was prepared to transfect 3 separate wells as technical replicates.

Thus, each well in the 48 well plate received a 40 μL transfection mixture consisting of: 16 μL (psiCHECK and activator plasmids in OptiMEM), 4 μL (50× Cond-siRNA in 1×PBS buffer), and 20 μL (1:50 dilution L2K). PBS was Phosphate Buffered Saline without calcium or magnesium treated with DEPC (diethyl pyrocarbonate) to remove any RNAse activity.

Immediately prior to transfection, the medium in each well was replaced with 160 μL of fresh medium, then added the 40 μL transfection mixture for a final volume of 200 μL/well with: 40 nanograms (ng) psiCHECK-2 dual luciferase reporter plasmid, 120 ng pBluescript or activator-expressing plasmid, and Cond-siRNA complexes at the indicated concentrations.

### Two-Step Transfection Protocol:

This protocol was used to generate data for FIGS. 5i-5k and FIG. 9a. Transfection 1 with target and activator plasmids, time—8 hours

For the two-step transfections, a master mix of the psiCHECK (Promega Corporation) reporter plasmid in Opti-MEM (Thermo Fisher Scientific) was created. This master mix was separated into aliquots and either the pBluescript (Agilent) control or one of the activator plasmids were then added. A 1:50 dilution of Lipofectamine 2000 (L2K) was added at 1:1 volume ratio with the plasmid mixtures to achieve the manufacturer recommended dilution of 1:100 L2K and incubated at room temperature to form lipoplexes according to the manufacturer's recommendation.

For each experimental condition, enough mixture was prepared to transfect 3 separate wells as technical replicates. Thus, each well in the 48 well plate received a 40 μL transfection mixture consisting of: 20 μL (psiCHECK and activator plasmids in Opti-MEM), and 20 μL (1:50 dilution L2K).

Immediately prior to transfection, the medium in each well was replaced with 160 μL of fresh medium, then the 40 μL transfection mixture was added for a final volume of 200 μL/well containing: 40 nanograms (ng) psiCHECK-2 dual luciferase reporter plasmid, and 120 ng pBluescript or activator-expressing plasmid.

The transfection mixture was removed and gently washed with media after six hours for about 2 hours. 160 ul fresh medium was added to each well and cell incubation continued until the second transfection.

Transfection 2 With Cond-siRNA Complexes, time—0

At 8 hours after transfection 1, Cond-siRNAs were transfected in varying concentrations as specified in the experiment using RNAiMAX reagent (ThermoFisher). For each experimental condition enough of each concentration of Cond-siRNA was prepared for technical triplicates of each target/activator combination in PBS. Each Cond-siRNA dilution was mixed with an equal volume of 1:50 RNAiMAX in OptiMEM and incubated at room temperature to form lipoplexes according to the manufacturer's instructions. Specifically, each well in the 48 well plate received a 40 μL transfection mixture consisting of: 20 μL Cond-siRNA at 10× final concentration (8 μL of PBS+12 μL of Opti-MEM), and 20 μL 1:50 dilution RNAiMAX.

### Maintenance

Time 0 is marked by the addition of the co-transfection mixture to cells for the single-step protocol, and as the time of addition of the Cond-siRNA complexes (transfection #2) for the two-step protocol. Medium was replaced at 18 hours post-transfection, at least once each following day, and 6 hours before lysate preparation.

### Lysate Preparation

At the designated time point for each experiment, 48 well plates were removed. Medium was carefully aspirated from each well. The wells were then washed once with 1×PBS and aspirated dry. 100 μL of 1× Promega Passive Lysis Buffer was added to each well. The plates were then covered in aluminum foil and either frozen at −80° C. or placed on a shaker for gentle agitation (˜70 rpm) at room temperature for ˜30 min. If frozen, cells were thawed on a shaker with gentle agitation for at least 30 min prior to dual luciferase assay. Before the assay, a visual inspection of the wells was used to ensure cells were well lysed.

### Assay

Cell lysates were assayed using the Dual-Luciferase Reporter Assay Kit (Promega) according to the manufacturer's instructions. Renilla luciferase values were normalized to Firefly luciferase in each technical replicate (each well). Technical triplicates within the experiment were averaged to obtain a single biological replicate value. All graphs represent the results of at least three independent biological replicate experiments.

### Example 11: Molecular Dynamics Simulations

Atomistic models of Cond-siRNAs were built using Nucleic Acid Builder⁴⁹ and custom scripts, and edited with the Accelrys (now BIOVIA, a division of Dassault Systems) Cerius⁴⁵ package to create appropriate chemical modifications.

A hybrid force field (FF) was created by combining previous Amber force field parameters reported for RNA⁵⁰, 2′-O-methyl⁵¹, LNA⁵², and phosphorothioate⁵³ modifications. Prior reports did not give a parameter set for LNA thymidine. FF parameters for the LNA sugar ring were derived from the LNA force field, and parameters for the base were derived from the Amber03 force field. Charges were calculated using the RESP ESP charge Derive (RED) server (q4md-forcefieldtools.org/REDServer/). FF parameters for non-DNA components such as the C₃ linker, terminal amine modifications, and terminal PEG linkers were taken from the GAFF FF⁵⁴. All structures were placed in a periodic box with 15 Å of spacing on each side and then solvated with TIP3 waters⁵⁵. Mg²⁺ ions were first added to neutralize half of the charge, and then Na⁺ was added to neutralize the second half. Finally, additional Na⁺ and Cl-ions were added to a concentration of 150 mM.

Molecular Dynamics simulations were run using LAMMPS13 GPU compatible release (Dec. 21, 2016) on nvidia K80 GPUs. Structures were minimized first with the steepest descent, then with conjugate gradient algorithms for 500 steps, and then equilibrated by MD simulations 310 K using the NVT ensemble over the course of 10 ps using a 1 fs timestep. The resulting structures then underwent 10 ps of NPT MD at 310 K, 350 atm to relax the periodic box and ensure positive net pressure. For NVT simulations, a Nose-Hoover thermostat was used with 100 fs time constant. For NPT simulations, a Nose-Hoover barostat was used with 1 ps time constant. Equilibrated structures then underwent 20 ns of MD at 310° K (NVT ensemble, 1 fs time step).

To obtain the structures presented in FIG. 9, the structure with the lowest potential energy from the MD trajectory was extracted and conjugate gradient energy minimization was applied for 500 steps. Constructs were visualized using the UCSF Chimera package⁵⁷. Helical parameters were calculated from simulation trajectories using the X3DNA⁵⁸.

### REFERENCES

1. Seeman, N. C. DNA in a material world. Nature 421, 427-431 (2003).
2. Guo, P. The emerging field of RNA nanotechnology. Nat Nano 5, 833-842 (2010).
3. Chen, Y.-J., Groves, B., Muscat, R. A. & Seelig, G. DNA nanotechnology from the test tube to the cell. Nat Nano 10, 748-760, doi:10.1038/nnano.2015.195 (2015).
4. Benenson, Y. Biomolecular computing systems: principles, progress and potential. Nat Rev Genet 13. 455-468, doi:http://www.nature.com/nrg/journal/v13/n7/suppinfo/nrg3197_S1.html (2012).
5. Yurke, B., Turberfield, A. J., Mills, A. P., Simmel, F. C. & Neumann, J. L. A DNA-fueled molecular machine made of DNA. Nature 406, 605-608, doi:nature.com/nature/journal/v406/n6796/suppinfo/406605a0_S1.html (2000).
6. Srinivas, N. et al. On the biophysics and kinetics of toehold-mediated DNA strand displacement. Nucleic Acids Research, doi:10.1093/nar/gkt801 (2013).
7. Green, A. A. et al. Complex cellular logic computation using ribocomputing devices. Nature 548, 117-121, doi:10.1038/nature23271nature.com/nature/journal/v548/n7665/abs/nature23271.html#supplementaryinformation (2017).
8. Groves, B. et al. Computing in mammalian cells with nucleic acid strand exchange. Nat Nano 11, 287-294, doi:10.1038/nnano.2015.278nature.com/nnano/journal/v11/n3/abs/nnano.2015.278.html#supplementaryinformation (2016).
9. Setten, R. L., Rossi, J. J. & Han, S. P. The current state and future directions of RNAi based therapeutics Nature Reviews Drug Discovery, In press (2019).
10. Bobbin, M. L. & Rossi, J. J. RNA Interference (RNAi)-Based Therapeutics: Delivering on the Promise? Annual Review of Pharmacology and Toxicology 56, 103-122, doi:10.1146/annurev-pharmtox-010715-103633 (2016).
11. Adams, D. et al. Patisiran, an RNAi Therapeutic, for Hereditary Transthyretin Amyloidosis. New England Journal of Medicine 379, 11-21, doi:10.1056/NEJMoa1716153 (2018).
12. Benenson, Y., Gil, B., Ben-Dor, U., Adar, R. & Shapiro, E. An autonomous molecular computer for logical control of gene expression. Nature 429, 423-429, doi:nature.com/nature/journal/v429/n6990/suppinfo/nature02551_S1.html (2004).
13. Kumar, D., Kim, S. H. & Yokobayashi, Y. Combinatorially Inducible RNA Interference Triggered by Chemically Modified Oligonucleotides. Journal of the American Chemical Society 133, 2783-2788, doi:10.1021/ja1107436 (2011).
14. Han, S. P., Barish, R. D. & Goddard, W. A. (Google Patents, 2015).
15. Han, S.-P., Goddard III, W. A., Scherer, L. & Rossi, J. J. Signal activatable constructs and related components compositions methods and systems. U.S. Pat. No. 9,725,715B2 (2015).
16. Hochrein, L. M., Ge, T. J., Schwarzkopf, M. & Pierce, N. A. Signal Transduction in Human Cell Lysate via Dynamic RNA Nanotechnology. ACS Synthetic Biology, doi:10.1021/acssynbio.8b00424 (2018).
17. Hochrein, L. M., Schwarzkopf, M., Shahgholi, M., Yin, P. & Pierce, N. A. Conditional Dicer Substrate Formation via Shape and Sequence Transduction with Small Conditional RNAs. Journal of the American Chemical Society 135, 17322-17330, doi:10.1021/ja404676x (2013).
18. Bindewald, E. et al. Multistrand Structure Prediction of Nucleic Acid Assemblies and Design of RNA Switches. Nano Letters 16. 1726-1735, doi:10.1021/acs.nanolett.5b04651 (2016).
19. Chatterjee, G., Chen, Y.-J. & Seelig, G. Nucleic Acid Strand Displacement with Synthetic mRNA Inputs in Living Mammalian Cells. ACS Synthetic Biology, doi:10.1021/acssynbio.8b00288 (2018).
20. Li, X., Yang, X., Qi, J. & Seeman, N. C. Antiparallel DNA Double Crossover Molecules As Components for Nanoconstruction. Journal of the American Chemical Society 118, 6131-6140, doi:10.1021/ja9601620 (1996).
21. Ha, M. & Kim, V. N. Regulation of microRNA biogenesis. Nature Reviews Molecular Cell Biology 15, 509, doi:10.1038/nrm3838 (2014).
22. MacRae, I. J. et al. Structural Basis for Double-Stranded RNA Processing by Dicer. Science (New York, N.Y.) 311, 195-198, doi:10.1126/science. 1121638 (2006).
23. Kim, D.-H. et al. Synthetic dsRNA Dicer substrates enhance RNAi potency and efficacy. Nat Biotech 23, 222-226, doi:nature.com/nbt/journal/v23/n2/suppinfo/nbt1051_S1.html (2005).
24. Khvorova, A. & Watts, J. K. The chemical evolution of oligonucleotide therapies of clinical utility. Nat Biotech 35, 238-248, doi:10.1038/nbt.3765 (2017).
25. Srinivas, N. et al. On the biophysics and kinetics of toehold-mediated DNA strand displacement. Nucleic Acids Research 41, 10641-10658, doi:10.1093/nar/gkt801 (2013).
26. ORBAN, T. I. & IZAURRALDE, E. Decay of mRNAs targeted by RISC requires XRN1, the Ski complex, and the exosome. RNA (New York, N.Y.) 11, 459-469, doi:10.1261/rna.7231505 (2005).
27. Hope, T. J. & Trono, D. Structure, Expression, and Regulation of the HIV Genome, <http://hivinsite.ucsf.edu/InSite?page=kb-00&doc=kb-02-01-02> (2000).
28. Kundu, M. & Liu, P. P. Function of the inv (16) fusion gene CBFB-MYH11. Current Opinion in Hematology 8, 201-205 (2001).
29. Look, A. T. Oncogenic Transcription Factors in the Human Acute Leukemias. Science (New York, N.Y.) 278, 1059-1064, doi:10.1126/science.278.5340.1059 (1997).
30. Plimpton, S. Fast Parallel Algorithms for Short-Range Molecular Dynamics. Journal of Computational Physics 117, 1-19, doi:dx.doi.org/10.1006/jcph. 1995.1039 (1995).
31. Condon, D. E. et al. Optimization of an AMBER Force Field for the Artificial Nucleic Acid, LNA, and Benchmarking with NMR of L(CAAU). The Journal of Physical Chemistry B 118, 1216-1228, doi:10.1021/jp408909t (2014).
32. Lind, K. E., Sherlin, L. D., Mohan, V., Griffey, R. H. & Ferguson, D. M. in Molecular Modeling of Nucleic Acids Vol. 682 ACS Symposium Series Ch. 3, 41-54 (American Chemical Society, 1997).
33. Aduri, R. et al. AMBER Force Field Parameters for the Naturally Occurring Modified Nucleosides in RNA. Journal of Chemical Theory and Computation 3, 1464-1475, doi:10.1021/ct600329w (2007).
34. Iwamoto, N. et al. Control of phosphorothioate stereochemistry substantially increases the efficacy of antisense oligonucleotides. Nat Biotech advance online publication, doi:10.1038/nbt.3948nature.com/nbt/journal/vaop/ncurrent/abs/nbt.3948.html#supplementaryinformation (2017).
35. Glaser, S. P. et al. Anti-apoptotic Mcl-1 is essential for the development and sustained growth of acute myeloid leukemia. Genes & Development 26, 120-125, doi:10.1101/gad. 182980.111 (2012).
36. Collingwood, M. A. et al. Chemical Modification Patterns Compatible with High Potency Dicer-Substrate Small Interfering RNAs. Oligonucleotides 18, 187-200, doi:10.1089/oli.2008.0123 (2008).
37. Yin, P. & Pierce, N. A. Triggered RNAi. U.S. Pat. No. 8,318,921B2 (2008).
38. Lee, H. Y., Zhou, K., Smith, A. M., Noland, C. L. & Doudna, J. A. Differential roles of human Dicer-binding proteins TRBP and PACT in small RNA processing. Nucleic Acids Research, doi:10.1093/nar/gkt361 (2013).
39 Han, S. P., Goddard, W. A., SCHERER, L. & Rossi, J. J. (Google Patents, 2015).
40. Silverman, S. K. Control of macromolecular structure and function using covalently attached double-stranded DNA constraints. Molecular BioSystems 3, 24-29, doi:10.1039/B614116A (2007).
41. Engelen, W., Janssen, B. M. G. & Merkx, M. DNA-based control of protein activity. Chemical Communications 52, 3598-3610, doi:10.1039/C5CC09853J (2016).
42. Mukherjee, P., Leman, L. J., Griffin, J. H. & Ghadiri, M. R. Design of a DNA-Programmed Plasminogen Activator. Journal of the American Chemical Society 140, 15516-15524, doi:10.1021/jacs.8b10166 (2018).
43. Colasanti, A. V., Lu, X.-J. & Olson, W. K. Analyzing and Building Nucleic Acid Structures with 3DNA. e4401, doi:doi:10.3791/4401 (2013).
44. Zadeh, J. N. et al. NUPACK: Analysis and design of nucleic acid systems. Journal of Computational Chemistry 32, 170-173, doi:10.1002/jcc.21596 (2011).
45. Mathews, D. H., Sabina, J., Zuker, M. & Turner, D. H. Expanded sequence dependence of thermodynamic parameters improves prediction of RNA secondary structure11 Edited by I. Tinoco. Journal of Molecular Biology 288, 911-940, doi:doi.org/10.1006/jmbi.1999.2700 (1999).
46. Camacho, C. et al. BLAST+: architecture and applications. BMC Bioinformatics 10, 421-421, doi:10.1186/1471-2105-10-421 (2009).
47. Tolstrup, N. et al. OligoDesign: optimal design of LNA (locked nucleic acid) oligonucleotide capture probes for gene expression profiling. Nucleic Acids Research 31, 3758-3762 (2003).
48. Scherer, L. J., Frank, R. & Rossi, J. J. Optimization and characterization of tRNA—shRNA expression constructs. Nucleic acids research 35, 2620-2628, doi:10.1093/nar/gkm103 (2007).
49. Macke, T. J. & Case, D. A. in Molecular Modeling of Nucleic Acids Vol. 682 ACS Symposium Series Ch. 24, 379-393 (American Chemical Society, 1997).
50. Duan, Y. et al. A point-charge force field for molecular mechanics simulations of proteins based on condensed-phase quantum mechanical calculations. Journal of Computational Chemistry 24, 1999-2012, doi:10.1002/jcc.10349 (2003).
51. Aduri, R. et al. AMBER Force Field Parameters for the Naturally Occurring Modified Nucleosides in RNA. Journal of Chemical Theory and Computation 3, 1464-1475, doi:10.1021/ct600329w (2007).
52. Condon, D. E. et al. Optimization of an AMBER Force Field for the Artificial Nucleic Acid, LNA, and Benchmarking with NMR of L(CAAU). The Journal of Physical Chemistry B 118, 1216-1228, doi:10.1021/jp408909t (2014).
53. Lind, K. E., Sherlin, L. D., Mohan, V., Griffey, R. H. & Ferguson, D. M. in Molecular Modeling of Nucleic Acids Vol. 682 ACS Symposium Series Ch. 3, 41-54 (American Chemical Society, 1997).
54. Wang, J., Wolf, R. M., Caldwell, J. W., Kollman, P. A. & Case, D. A. Development and testing of a general amber force field. Journal of Computational Chemistry 25, 1157-1174, doi:10.1002/jcc.20035 (2004).
55. Mark, P. & Nilsson, L. Structure and Dynamics of the TIP3P, SPC, and SPC/E Water Models at 298K. The Journal of Physical Chemistry A 105, 9954-9960, doi:10.1021/jp003020w (2001).
56. Plimpton, S. Fast Parallel Algorithms for Short-Range Molecular Dynamics. Journal of Computational Physics 117, 1-19, doi:dx.doi.org/10.1006/jcph. 1995.1039 (1995).
57. Pettersen, E. F. et al. UCSF Chimera-A visualization system for exploratory research and analysis. Journal of Computational Chemistry 25, 1605-1612, doi:10.1002/jcc.20084 (2004).
58. Colasanti, A. V., Lu, X.-J. & Olson, W. K. Analyzing and Building Nucleic Acid Structures with 3DNA. e4401, doi: doi:10.3791/4401 (2013).
59. Mathews et al., Expanded Sequence Dependence of Thermodynamic Parameters Improves Prediction of RNA Secondary Structure, J. Mol. Biol. (1999) 288:911-940.
60. U.S. Pat. No. 9,029,524.
61. Meggers, et al., Acc. Chem. Res. (2010) 43 (8): 1092-1102.
62. Schlegel et al., J. Am. Chem. Soc. (2017) 139 (25): 8537-8546.

1.-12. (canceled)

13. A method of reducing expression of a target gene in a cell, comprising:
contacting the cell with an effective amount of a conditionally activatable small interfering RNA (Cond-siRNA) construct comprising a sensor strand, a core strand, and a guide strand, wherein:
(i) the sensor strand and the core strand form a sensor duplex,
(ii) the guide strand and the core strand form an RNAi duplex, and
(iii) the sensor duplex is attached to the RNAi duplex to form a single structure; wherein an input strand binds to the sensor strand causing displacement of the sensor strand from the Cond-siRNA, thereby activating the RNAi in the Cond-siRNA construct; and wherein
(a) 50% or less of the backbone positions in the sensor duplex domain of the sensor strand are phosphorothioate (PS) connections; and/or
(b) the terminal base on the 5′ terminus of the core strand is a 2′-F or 2′-O-methyl, and/or the terminal base on the 3′ terminus of the core strand is a 2′-F or 2′-O-methyl; and/or
(c) the three bases of the core strand base-paired with the 3′ terminus of the guide strand have a M*+*M pattern, where M is a 2′-F or 2′-O-methyl, * is a PS backbone connection, and + is an LNA base or other 2′-4′ bridged base.

14. The method of claim 13, wherein the sensor strand has an overhang that is not complementary to the core strand, and wherein the overhang on the sensor strand is capable of complementary binding to an input strand to form a toehold, thereby causing the displacement of the sensor strand from the core strand.

15. The method of claim 13, wherein the Cond-siRNA comprises one or more of the following chemical modifications:
A. the duplex domain of the sensor strand has one or more of the following features:
a. 50% or more of the bases are chemically modified to resist nuclease degradation or increase the melting temperature of the duplex (Tm);
b 100% of the bases are chemically modified to resist nuclease degradation and increase Tm; and
c. about 10%-50% of the bases are locked nucleic acid (LNA) or other chemically modified base with 2′-4′ bridging modifications that substantively increase the Tm;
B. the 5′ and 3′ termini of the core strand have one or more of the following features:
a the three terminal bases on the 5′ have the pattern MRM, where M is a modified base (2′-O-methyl, 2′-F), and R is an RNA base;
b the three terminal bases on the 3′ have the pattern MRM, where M is a modified base (2′-O-methyl, 2′-F), and R is an RNA base;
c. the three terminal bases on the 3′ and 5′ do not have consecutive PS backbone modifications;
d. the portions of the core strand that are base-paired with the sensor strand have an alternating chemical modification pattern (MR)n;
e. the above where M is a chemically modified base that does not decrease Tm of the duplex when compared to the equivalent RNA base;
f. any combination where both the 5′ and the 3′ ends of the core strand have at least one of the features from a-e; and
g. the 3′ and 5′ regions of the core strand that are base-paired with the sensor strand are:
(a) entirely made out of the pattern (M)n, where M is a 2′-O-methyl or 2′-F modified base, or
(b) at least 50% of the bases in this area are 2′-O-methyl or 2′ F, and, up to 30%, 50%, 80% or 100% of the backbone connections are not phosphorothioate;
C. a core strand where the three bases base-paired with 3′ terminus of the guide strand have one or more of the following features:
a. A M*+*+ pattern;
b. A +*+*+ pattern;
c. A R*+*M pattern, where R is an RNA base;
d. A R*+*+ pattern;
e. A +*M*M pattern; and
f. the patterns in a-e, where * can be either a PS backbone connection or an unmodified (phosphodiester) backbone connection; and
D. a guide strand with one or more of the following features:
a. 30% to 95% of the bases are chemically modified bases (2′-O-methyl, 2′-F, LNA, 2′-4′ bridged bases);
b. where the two terminal bases on the 5′ are chemically modified;
c. where the two terminal bases on the 5′ have at least one LNA;
d. where the two terminal bases on the 5′ are connected by a PS backbone;
e. where the two terminal bases on the 3′ are both chemically modified;
f. where ˜5% to 50% of backbone connections are PS; and
g. where the bases flanking the site of Dicer cleavage are not chemically modified.

16. The method of claim 13, wherein the duplex domain of the sensor strand has LNA modification, 2′-O-methyl modification, or both.

17. The method of claim 13, wherein the duplex domain of the sensor strand does not have phosphorothioate (PS) modifications.

18. The method of claim 14, wherein the Cond-siRNA comprises chemical modifications in the toehold domain of the sensor strand, including LNA modification, 2′-O-methyl modification, PS modification, or a combination thereof.

19. The method of claim 13, wherein the sensor duplex is 23 bp.

20. The method of claim 13, wherein the RNAi duplex is 23 bp.

21. The method of claim 13, wherein the input strand is an mRNA, a miRNA, or a non-coding RNA.

22. The method of claim 13, wherein the effective amount of the Cond-siRNA comprises a concentration of 0.016 nM to 2 nM.

23. The method of claim 13, wherein the Cond-siRNA has a knockdown activity of at least 90% when in an “ON” state.

24. The method of claim 23, wherein the effective amount comprises a concentration of 2 nM.

25. The method of claim 13, wherein the Cond-siRNA has a knockdown activity of less than 25% when in an “OFF” state.

26. The method of claim 25, wherein the effective amount comprises a concentration of 2 nM.

27. The method of claim 13, wherein the input strand is a cellular RNA transcript.

28. The method of claim 13, wherein the guide strand is capable of hybridizing to at least a portion of the target gene mRNA.

29. The method of claim 13, wherein activating the RNAi comprises:
digestion by one or more endogenous nucleases of 5′ and 3′ overhangs of the core strand in the RNAi duplex following displacement of the sensor strand, thereby producing a Dicer-processable duplex.

30. The method of claim 13, wherein the cell is a mammalian cell.
